(19) United States
(12) Patent Application Publication
(10) Pub. No.: US 2025/0248945 A1
(43) Pub. Date: Aug. 7, 2025

(54) BIODEGRADABLE LIPIDOIDS AND COMPOSITIONS AND METHODS OF USE THEREOF FOR TARGETED DELIVERY

(71) Applicant: The Trustees Of The University Of Pennsylvania, Philadelphia, PA (US)

(72) Inventors: Michael J. Mitchell, Philadelphia, PA (US); Lulu Xue, Philadelphia, PA (US)

(21) Appl. No.: 18/856,855

(22) PCT Filed: Apr. 13, 2023
(86) PCT No.: PCT/US2023/065729

§ 371 Date: Oct. 14, 2024

Related U.S. Application Data
(60) Provisional application No. 63/331,060, filed on Apr. 14, 2022.

(51) Int. Cl. A61K 9/51; A61K 31/7105; A61K 31/711; A61K 39/385; C07C 229/16; C07D 295/13

(52) U.S. Cl. CPC A61K 9/5123; A61K 31/7105; A61K 31/711; A61K 39/385; C07C 229/16; C07D 295/13

(57) ABSTRACT

The disclosure relates, in part, to biodegradable lipid nanoparticles (LNPs) comprising biodegradable lipidoid compounds and compositions thereof. In certain embodiments, the LNPs selectively target a cell of interest (e.g., an immune cell, stem cell, bone cell, blood cell, fat cell, endothelial cell, cancer cell, tissue cell, nerve cell, epithelial cell, connective tissue cell, and muscle cell, inter alia). In certain embodiments, the disclosure further relates to methods for in vivo delivery of therapeutic agents for the treatment, prevention, and/or amelioration of diseases or disorders using the LNPs of the disclosure.

### CROSS-REFERENCE TO RELATED APPLICATIONS

The present application claims priority under 35 U.S.C. § 119(e) to U.S. Provisional Patent Application No. 63/331,060, filed Apr. 14, 2022, which is incorporated herein by reference in its entirety.

### STATEMENT REGARDING FEDERALLY SPONSORED RESEARCH OR DEVELOPMENT

This invention was made with government support under TR002776 awarded by the National Institutes of Health. The government has certain rights in the invention.

### BACKGROUND

With the development of new technologies (such as, but not limited to, RNA therapeutics, gene therapy, and gene editing technologies), it is necessary to address the challenge of delivering them to cells in a precise and efficient way. Currently, there are three FDA approved/EUA products that utilize lipid nanoparticles (LNPs)—Onpattro (siRNA) and the Pfizer and Moderna mRNA COVID-19 vaccines.

However, the development of mRNA-LNPs system is challenging. For example, one major challenge in the development of mRNA-LNPs systems is the identification of safety and efficacy, which support a sufficiently broad therapeutic index for chronic indications. Unfortunately, improvements in LNPs delivery potency do not always result in a desired therapeutic index since the restriction and reduction in tolerated dose levels. Although non-hydrolysable lipid-like materials have been proved to exhibit a satisfied delivery efficacy, the delivery toxicity still remains. Based on this, some studies developed degradable LNP systems for in vivo RNA therapeutics delivery, but the delivery potency compared with benchmark LNPs, such as C12-200, was not high enough for low dosing and long-term treatment. Therefore, it is still urgent to develop novel LNP delivery systems with both high delivery efficacy and low toxicity.

Thus, there is a need in the art for LNP delivery systems with high delivery efficacy and low toxicity to deliver RNA therapeutics, gene therapy, gene editing technologies, and so forth, in a precise and efficient way to a cell of interest. The present invention satisfies this unmet need.

### BRIEF SUMMARY

The present disclosure provides in one aspect certain biodegradable lipidoids, as well as compositions and methods of use using the same for targeted delivery.

In certain embodiments, the disclosure provides a compound of Formula (I), or a salt, solvate, stereoisomer, isotopologue, or derivative thereof.

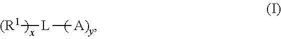

wherein each occurrence of A is independently

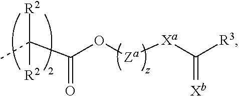

and
wherein R¹, x, A, y, R², Z^{a}, z, X^{a}, X^{b}, and R³ are defined elsewhere herein.

In certain embodiments, the disclosure provides a biodegradable lipid nanoparticle (LNP). In certain embodiments, the LNP comprises at least one compound of the disclosure.

In certain embodiments, the LNP comprises at least one neutral phospholipid. In certain embodiments, the neutral phospholipid is present in a concentration range of about 5 mol % to about 45 mol %. In certain embodiments, the LNP comprises at least one cholesterol lipid. In certain embodiments, the total cholesterol lipid is in a concentration range of about 5 mol % to about 55 mol %. In certain embodiments, the LNP comprises at least one polyethylene glycol (PEG) or PEG-conjugated lipid. In certain embodiments, the PEG or PEG-conjugated lipid is in a concentration range of about 0.5 mol % to about 12.5 mol %.

In certain embodiments, the disclosure provides a composition comprising at least one biodegradable LNP of the disclosure.

In certain embodiments, the disclosure provides a method of delivering an agent to a subject in need thereof. In certain embodiments, the method comprises administering a therapeutically effectively amount of at least one biodegradable LNP of the disclosure or a composition comprising the same to the subject.

In certain embodiments, the disclosure provides a method of delivering an agent to a liver. In certain embodiments, the method comprises administering a therapeutically effectively amount of at least one biodegradable LNP of the disclosure or a composition comprising the same.

In certain embodiments, the disclosure provides a method of treating or preventing at least one disease or disorder in a subject in need thereof. In certain embodiments, the method comprises administering a therapeutically effectively amount of at least one biodegradable LNP of the disclosure or a composition comprising the same to the subject.

In certain embodiments, the disclosure provides a method of inducing an immune response in a subject in need thereof. In certain embodiments, the method comprises administering a therapeutically effectively amount of at least one biodegradable LNP of the disclosure or a composition comprising the same to the subject

### BRIEF DESCRIPTION OF THE FIGURES

The drawings illustrate generally, by way of example, but not by way of limitation, various embodiments of the present application.

FIGS. 1A-1G depict a schematic representation of design and synthesis of a combinatorial library of biodegradable lipidoids and lipid nanoparticles (LNPs) for nucleic acid delivery. FIG. TA depicts a schematic representation of exemplary biodegradable lipid nanoparticles (BLNPs) that are formulated via microfluidic device with biodegradable ionizable lipids, helper lipid (DOPE), cholesterol, and PEG-lipid (C14PEG2000). FIG. 1B depicts a representative example of BLNPs analyzed with Cryogenic transmission electron microscopy (cryo-TEM) for their size and structural analysis. Obtained BLNPs possess a flower-like morphology. Scale bar: 150 nm. FIG. 1C depicts representative hydrodynamic diameter of exemplary BLNPs shown in FIG. 1B. FIG. 1D depicts schematic representations of 12 exemplary amines and 12 exemplary biodegradable tails used for the syntheses of 144 exemplary biodegradable lipidoids. FIGS. 1E-1G depict schematic representations of 9 exemplary biodegradable lipidoids.

FIGS. 2A-2E depict representative structure-activity relationships of BLNPs for luciferase mRNA delivery in vitro. FIG. 2A depicts a representative heat map of luciferase expression following treatment of Hela cells with BLNPs (10 ng luciferase mRNA, 5000 cells, n≥3 biological independent samples). Relative light units (RLU) of >300 was counted for hit rate calculation. FIG. 2B depicts a representative relative hit rate of BLNPs with different numbers of N per lipidoids. FIG. 2C depicts a representative relative hit rate of BLNPs with different tails substitution number. FIG. 2D a representative relative hit rate of BLNPs with different tails length. FIG. 2E a representative luciferase intensity fold increase of BLNPs with tunable tails structures to benchmark (C12-200). BLNPs having higher luciferase transfection efficiency than benchmark (C12-200) were chosen for comparison.

FIGS. 3A-3E depict a representative example of BLNPs (i.e., 306-Hb7b2) that facilitated nanoparticle uptake, EGFP mRNA transfection and endosomal escape comparing with benchmark (C12-200) in vitro. FIG. 3A depicts a representative LNPs uptake and EGFP expression on Hela cells treated by C12-200 (Top) and 306-HB7b2 (Bottom) LNPs carrying EGFP mRNA. Biodegradable 306-HB7b2 LNPs showed higher cellular uptake and stronger EGFP expression. DiD fluorescence dye was used to label LNPs at a concentration of 0.2%. Samples were incubated for 3 h before imaging. Scale bar: 20 μm. FIG. 3B depicts a representative qualification of LNPs uptake by measuring DiD intensity from flow cytometry. 306-HB7b2 exhibited much higher DiD intensity than C12-200. FIG. 3C depicts a representative qualification of EGFP-LNPs expression by measuring EGFP intensity from flow cytometry. 306-HB7b2 exhibited much higher EGFP transfection efficiency than C12-200. FIGS. 3D-3E depict representative endosomal escape of luciferase mRNA-loaded C12-200 (Top) and 306-HB7b2 (Bottom) LNPs. Hela cells were treated with 0.5 g/mL luciferase mRNA encapsulated in LNPs as indicated for 3 h. DiO was used to label the LNPs at a concentration of 1%. Lysotracker was used to stain the endosome for 1 h, while Hoechst were used to stain the nucleus for 5 min. Samples and dye markers were washed off before imaging. 306-HB7b2 LNPs treated cells displayed weaker overlapping of green and red colors than C12-200 LNPs, demonstrating enhanced endosomal escape capability. Scale bar: 20 μm.

FIGS. 4A-4F depict representative structure-activity studies revealed that biodegradable lipid structure behaved much higher in vivo efficacy than benchmark (C12-200). FIG. 4A depicts representative in vivo evaluation of 9 top BLNPs and C12-200 at a luciferase mRNA dose (0.1 mg/kg). Bioluminescence images of whole bodies and various organs were recorded 12 h after i.v. injection of LNPs into C57BL/6 mice. These 9 top BLNPs showed higher mRNA transfection in vivo compared with C12-200. H, heart; Li, liver; S, spleen; Lu, lung; K, kidney. FIG. 4B depicts a representative whole body radiance of mice shown in FIG. 4A. FIG. 4C depicts a representative mRNA expression in liver by BLNPs and benchmark shown in FIG. 4A. All data are presented as mean s.d. (n=3 biologically independent mice). FIG. 4D depicts a representative mRNA expression in spleen by BLNPs and benchmark shown in FIG. 4A. All data are presented as mean±s.d. (n=3 biologically independent mice). FIG. 4E depicts a representative mRNA expression in lung by BLNPs and benchmark shown in FIG. 4A. All data are presented as mean±s.d. (n=3 biologically independent mice). FIG. 4F depicts representative ELISA quantification of serum hEPO in C57BL/6 mice treated with lead BLNPs (306-HB7b2) and benchmark (C12-200) encapsulating hEPO mRNA (0.3 mg hEPO mRNA/kg body weight, 12 h). Statistical significance was calculated using Multiple t test with unpaired design. ***P<0.001.

FIGS. 5A-5B depict a representative liver toxicity assay after injection of LNPs encapsulating luciferase-encoding mRNA. FIG. 5A depicts a representative alanine transaminase (ALT) quantification (± standard deviation) for control, two representative BLNPs (306-HB6b and 306-HB7b2 LNPs), and benchmark (C12-200 LNPs). n=5 biological animals. FIG. 5B depicts a representative aspartate transaminase (AST) quantification (± standard deviation) for control, two representative BLNPs (306-HB6b and 306-HB7b2 LNPs), and benchmark (C12-200 LNPs). n=5 biological animals. C57BL/6J mice were dosed with 1.0 mg/kg luciferase mRNA LNPs, and liver enzymes were quantified 12 h after injection. Two representative BLNPs showed much lower liver toxicity than benchmark. Statistical significance was calculated using Multiple t test with unpaired design. ***P<0.001; **P<0.01.

FIGS. 6A-6D depict an exemplary hemolysis analysis of outperformer 306-HB7b2 LNP and benchmark (C12-200 LNP). FIG. 6A depicts a cartoon image of hemolysis performance of 306-HB7b2 LNP and C12-200 LNP. Red blood cell samples were treated by LNP or PBS or Triton X under pH=5.5 and pH=7.4, respectively. PBS treated group and 1× Triton X buffer treated groups were used as negative and positive control, respectively. n=3.

FIG. 6B depicts dose-dependent hemolysis of 306-HB7b2 LNP and C12-200 LNP treated groups. LNPs with dosage of 6 μM, 30 μM, and 60 μM were added into red blood cell samples for test from FIG. 6A. 306-HB7b2 LNP exhibited better hemolysis ability than benchmark C12-200 LNP, demonstrating greater membrane fusion performance for endosomal escape. pKa of 306-HB7b2 LNP (FIG. 6C) and C12-200 LNP (FIG. 6D) were tested by TNS measurements.

FIG. 7 depicts a heatmap of immunogenicity of 306-HB7b2 LNP and benchmark (C12-200 LNP). Cytokines and chemokines were measured in the serum of mice (n=3). 306-HB7b2 LNP exhibited much lower cytokines and chemokines level in the serum, demonstrating lower immunogenicity of degradable 306-HB7b2 LNP than benchmark (C12-200 LNP) for more efficient mRNA delivery. Statistical significance calculated using test with unpaired design was listed at the left side of heatmap. ***P<0.001: **P<0.01, *P<0.05.

FIGS. 8A-8F depict in vivo imaging of C57Bl/6 mice intravenously administered exemplary LNPs of the present disclosure comprising Luciferase mRNA, as compared to LNPs comprising C12-200, at 4 h and 24 h.

### DETAILED DESCRIPTION

The present disclosure is based, in part, on the unexpected discovery of biodegradable lipidic compounds having the structure of Formula (I). In one aspect, the present disclosure provides a lipid nanoparticle (LNP) that is biodegradable comprising at least one compound of the present disclosure. In various embodiments, the LNP comprises one or more compounds of the present disclosure in a concentration range of about 0.1 mol % to about 99.99 mol %. In some embodiments, the LNP comprises one or more compounds of the present disclosure in a concentration range of about 1 mol % to about 95 mol %. In some embodiments, the LNP comprises one or more compounds of the present disclosure in a concentration range of about 10 mol % to about 50 mol %.

In various embodiments, the LNP comprises at least one agent for delivery to a cell of interest. For example, such cells include, but are not limited to, a tissue cell or a muscle cell (e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, and so forth).

In another aspect, the present disclosure provides a LNP, comprising at least one compound of the present disclosure, that selectively targets a cell of interest and is formulated for in vivo stability as well as methods of use thereof for in vivo delivery of an encapsulated agent to the cell of interest. Exemplary agents that can be encapsulated in the compositions of the disclosure include, but are not limited to, diagnostic agents, detectable agents, and therapeutic agents. In certain embodiments, the present disclosure provides a composition comprising a LNP encapsulating a nucleic acid molecule (e.g., mRNA). In certain embodiments, the nucleic acid excludes nucleic acids that comprises a coding sequence for an editing enzyme. In certain embodiments, the nucleic acid excludes nucleic acids which comprises a coding sequence for a nuclease operably linked to sequences which direct expression thereof in a liver cell. In certain embodiments, the nucleic acid excludes an mRNA encoding a Cas9. In certain embodiments, the nucleic acid excludes nucleic acid that encode a synthetic or engineered nuclease, a zinc finger nuclease. a TAL-effector nuclease, or a meganuclease. In certain embodiments, the nucleic acid excludes nucleic acid molecules encoding a gene product or a transcript therefor operably linked to regulatory sequences which direct expression thereof.

In one aspect, the composition of the present disclosure comprises one or more LNP formulated for targeted delivery of an agent to a cell of interest (e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, and so forth).

In another aspect, the present disclosure provides a method of inducing an immune response in a subject in need thereof. In some embodiments, the method comprises administering a therapeutically effectively amount of at least one LNP or composition described herein to a subject. In some embodiments, the therapeutically effectively amount of at least one LNP or composition described herein induces an immune response against cancer in the subject.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y. or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” as used herein when referring to a measurable value, for example numerical values and/or ranges, such as an amount, a temporal duration, and the like, is meant to encompass variations of 20%, ±10%, ±5%, ±1%, or ±0.1% from the specified value, as such variations are appropriate to perform the disclosed methods. For example, “about 40 [units]” may mean within ±25% of 40 (e.g., from 30 to 50), within ±20%, ±15%, %, ±9%, 8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein.

The term “abnormal” when used in the context of organisms, tissues, cells or components thereof, refers to those organisms, tissues, cells or components thereof that differ in at least one observable or detectable characteristic (e.g., age, treatment, time of day, and so forth) from those organisms, tissues, cells or components thereof that display the “normal” (expected) respective characteristic. Characteristics which are normal or expected for one cell or tissue type, might be abnormal for a different cell or tissue type.

The term “adjuvant” as used herein is defined as any molecule to enhance an antigen-specific adaptive immune response.

A disease or disorder is “alleviated” if the severity of a sign or symptom of the disease or disorder, the frequency with which such a sign or symptom is experienced by a patient, or both, is reduced.

“Alkenyl” or “alkenyl group” refers to a straight or branched hydrocarbon chain having from 2 to 20 carbon atoms, and having one or more carbon-carbon double bonds. Each alkenyl group is attached to the rest of the molecule by a single bond. Alkenyl group comprising any number of carbon atoms from 2 to 20 are included. In some embodiments, the alkenyl is a C₂-C₂₀ alkenyl, a C₂-C₁₂ alkenyl, a C₂-C₁₀ alkenyl, a C₂-C₅ alkenyl, a C₂-C₆ alkenyl, a C₂-C₄ alkenyl, or a C₂-C₃ alkenyl. An alkenyl group comprising up to 20 carbon atoms is a C₂-C₂₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₂₀ alkenyl includes all the foregoing moieties, but also includes C₁₁, C₁₂, C₁₃, C₁₄, C₁₅, C₁₆, C₁₇, C₁₈, C₁₉, and C₂₀ alkenyls. Non-limiting examples of C₂-C₂₀ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl. 3-undecenyl. 4-undecenyl. 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

As used herein, the term “alkoxy” employed alone or in combination with other terms means, unless otherwise stated, refers to a group of the formula —ORₐ where Rₐ is an alkyl, alkenyl or alknyl group having from 1 to 20 carbon atoms, as defined above, connected to the rest of the molecule via an oxygen atom, such as, for example, methoxy, ethoxy, 1-propoxy, 2-propoxy (isopropoxy) and the higher homologs and isomers. Unless stated otherwise specifically in the specification, an alkoxy group can be optionally substituted.

As used herein, the term “alkyl,” or “alkyl group” by itself or as part of another substituent means, unless otherwise stated, a straight or branched chain hydrocarbon having from 1 to 20 carbon atoms. In some embodiments, the alkyl is a C₁-C₂₀ alkyl, a C₁-C₁₂ alkyl, a C₁-C₁₀ alkyl, a C₁-C₈ alkyl, a C₁-C₆ alkyl, a C₁-C₄ alkyl, or a C₁-C₃ alkyl. For example, an alkyl comprising up to 20 carbon atoms is a C₁-C₂₀ alkyl, an alkyl comprising up to 12 carbon atoms is a C₁-C₁₂ alkyl, an alkyl comprising up to 10 carbon atoms is a C₁-C₁₀ alkyl and an alkyl comprising up to 5 carbon atoms is a C₁-C₅ alkyl. A C₁-C₈alkyl includes C₈ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₈ alkyls and C₁-C₈ alkyls, but also includes C₇, C₈, C₉ and C₁₀ alkyls. Similarly, a C₁-C₂₀ alkyl includes all the foregoing moieties, but also includes C₁₁, C₁₂, C₁₃, C₁₄, C₁₅, C₁₆, C₁₇, C₁₈, C₁₉, and C₂₀ alkyls. Non-limiting examples include methyl, ethyl, n-propyl, isopropyl, n-butyl, isobutyl, sec-butyl, tert-butyl, n-pentyl, neopentyl, n-hexyl, n-heptyl, n-octyl, n-nonyl, n-decyl, n-undecyl, n-dodecyl, and cy clopropylmethyl. Unless stated otherwise specifically in the specification, an alkyl group can be optionally substituted.

“Alkylamino” refers to a group of the formula —NHRₐ or —NRₐRₐ where each Rₐ is, independently, an alkyl, alkenyl or alkynyl group as defined above containing 1 to 20 carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl group as defined above. A non-limiting example of an alkyl carbonyl is the methyl carbonyl (“acetal”) moiety. Alkylcarbonyl groups can also be referred to as “C_{w}-C_{z} acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₁-C₁₀ alkenyl, or C₁-C₁₀ alkynyl group as defined above. Unless stated otherwise specifically in the specification, an alkyl carbonyl group can be optionally substituted.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms.

“Alkynyl” or “alkynyl group” refers to a straight or branched hydrocarbon chain having from 2 to 20 carbon atoms, and having one or more carbon-carbon triple bonds. Each alkynyl group is attached to the rest of the molecule by a single bond. In some embodiments, the alkynyl is a C₂-C₂₀ alkynyl, a C₂-C₁₂ alkynyl, a C₂-C₁₀ alkynyl, a C₂-C₅ alkynyl, a C₂-C₆ alkynyl, a C₂-C₄ alkynyl, or a C₂-C₃ alkynyl. Alkynyl group comprising any number of carbon atoms from 2 to 20 are included. An alkynyl group comprising up to 20 carbon atoms is a C₂-C₂₀ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₂₀ alkynyl includes all the foregoing moieties, but also includes C₁₁, C₁₂, C₁₃, C₁₄, C₁₅, C₁₆, C₁₇, C₁₈, C₁₉, and C₂₀ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

The term “amino” refers to a group of the formula —NRₐRₐ, —NHRₐ, or —NH₂, where each Rₐ is, independently, an alkyl (e.g., aminoalkyl), alkenyl or alkynyl group as defined above containing 1 to 20 carbon atoms. Unless stated otherwise specifically in the specification, an alkylamino or aminoalkyl group can be optionally substituted.

As used herein, the terms “amino acid”, “amino acidic monomer”, or “amino acid residue” refer to any of the twenty naturally occurring amino acids including synthetic amino acids with unnatural side chains and including both D and L optical isomers.

The “aminoalkyl linker” or “aminoalkylenyl” as used herein refers to a bivalent, at least partially saturated, aliphatic diradical comprising at least one nitrogen atom. In certain embodiments, the nitrogen atom has a lone pair. In certain embodiments, the term may be regarded as a moiety derived from the corresponding aminoalkyl by removal of two hydrogen atoms from the same or different carbon atom and/or heteroatom(s). The terms “mono”, “di”, “tri”, “tetra”, “penta”, and the like, used in conjunction with the term “aminoalkyl linker” indicate the number of nitrogen atoms comprising the moiety (i.e., a triaminoalkyl linker comprises three nitrogen atoms).

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, prodrug, or other variant of the reference molecule.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present disclosure may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. k and l light chains refer to the two major antibody light chain isotypes.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequences or a partial nucleotide sequence encoding a protein that elicits an adaptive immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The terms “aralkenyl” or “arylalkenyl” refer to a radical of the formula —R_{b}—R_{c} where R_{b} is an alkenylene o group as defined above and R_{c} is one or more aryl radicals as defined above. Unless stated otherwise specifically in the specification, an aralkenyl group can be optionally substituted.

The terms “aralkyl” or “arylalkyl” refer to a radical of the formula —R_{b}—R_{c} where R_{b} is an alkylene group as defined above and R_{c} is one or more aryl radicals as defined above, for example, benzyl, diphenylmethyl and the like. Unless stated otherwise specifically in the specification, an aralkyl group can be optionally substituted.

The terms “aralkynyl” or “arylalkynyl” refer to a radical of the formula —R_{b}—R_{c} where R_{b} is an alkynylene group as defined above and R_{c} is one or more aryl radicals as defined above. Unless stated otherwise specifically in the specification, an aralkynyl group can be optionally substituted.

As used herein, the term “aromatic” refers to a carbocyclyl or heterocyclyl with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a hydrocarbon ring system, comprising hydrogen, 6 to 18 carbon atoms and at least one aromatic ring. For purposes of this disclosure, the aryl can be a monocyclic, bicyclic, tricyclic or tetracyclic ring system, which can include fused or bridged ring systems. For example, aryls include, but are not limited to, a biphenyl, or may be fused, such as naphthalene. Examples of aryl groups include benzyl, indacenyl, pyrenyl, triphenyl, phenyl, anthracyl, and naphthyl. Unless stated otherwise specifically in the specification, the term “aryl” is meant to include aryl groups that are optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyl, cycloalkenyl and cycloalkynyl as defined herein. Unless stated otherwise specifically in the specification, a carbocyclyl group can be optionally substituted.

The term “compound,” as used herein, unless otherwise indicated, refers to any specific chemical compound disclosed herein. In certain embodiments, the term also refers to stereoisomers and/or optical isomers (including racemic mixtures) or enantiomerically enriched mixtures of disclosed compounds.

The term “cyano” refers to a group of the formula —CN group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbomyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

Monocyclic cycloalkyls include, but are not limited to, cyclopropyl, cyclobutyl, cyclopentyl, cyclohexyl, cycloheptyl, and cyclooctyl. Dicyclic or polycyclic cycloalkyls include, but are not limited to, tetrahydronaphthyl, indanyl, and tetrahydropentalenyl, adamantyl and norbomyl. The term cycloalkyl includes “unsaturated nonaromatic carbocyclyl,” “carbocyclyl,” “carbocyclic ring,” “carbocycle,” or “nonaromatic unsaturated carbocyclyl” groups, both of which refer to a nonaromatic carbocycle as defined herein, which contains at least one carbon double bond or one carbon triple bond.

The term “cycloalkylene” or “cycloalkylenyl” as used herein refers to a bivalent saturated cycloalkyl radical (e.g.,

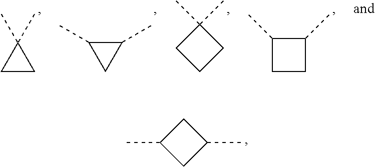

inter alia). In certain embodiments, the term may be regarded as a product of removal of two hydrogen atoms from the corresponding cycloalkane (e.g., cyclobutyl) by removal of two hydrogen atoms from the same (e.g.,

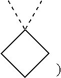

different (e.g.,

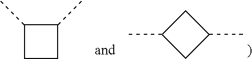

carbon atoms.

“Cycloalkenyl” refers to a stable non aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyls include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}-Rd where R_{b} is an alkylene, alkenylene, or alkynylene group as defined above and Rd is a cycloalkyl, cycloalkenyl, cycloalkynyl radical as defined above. Unless stated otherwise specifically in the specification, a cycloalkylalkyl group can be optionally substituted.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” or “therapeutically effective amount”, as used herein, means an amount which provides a therapeutic or prophylactic benefit. An “effective amount” or “therapeutically effective amount” of a compound is that amount of compound which is sufficient to provide a beneficial effect to the subject to which the compound is administered. For example, a “therapeutically effective amount” of the LNPs is the amount that is sufficient or effective to prevent or treat (delay or prevent the onset of, prevent the progression of, inhibit, decrease or reverse) a disease or condition, including alleviating symptoms of such diseases.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “halo” or “halogen” alone or as part of another substituent means, unless otherwise stated, a fluorine, chlorine, bromine, or iodine group.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

As used herein, the term “heteroalkyl” by itself or in combination with another term means, unless otherwise stated, a stable straight or branched chain alkyl group consisting of from 1 to 20 carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom can be optionally substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. The term heterocycloalkyl group can also be a C₂ heterocycloalkyl, C₂-C₃ heterocycloalkyl, C₂-C₄ heterocycloalkyl, C₂-C₅ heterocycloalkyl, C₂-C₆ heterocycloalkyl, C₂-C₇ heterocycloalkyl, C₂-C₈ heterocycloalkyl, C₂-C₉ heterocycloalkyl, C₂-C₁₀ heterocycloalkyl, C₂-C₁ heterocycloalkyl, and the like, up to and including a C₂-14₅ heterocycloalkyl. For example, a C₂ heterocycloalkyl comprises a group which has two carbon atoms and at least one heteroatom, including, but not limited to, aziridinyl, diazetidinyl, oxiranyl, thiiranyl, and the like. Alternatively, for example, a C₅ heterocycloalkyl comprises a group which has five carbon atoms and at least one heteroatom, including, but not limited to, piperidinyl, tetrahydropyranyl, tetrahydrothiopyranyl, diazepanyl, and the like. It is understood that a heterocycloalkyl group may be bound either through a heteroatom in the ring, where chemically possible, or one of carbons comprising the heterocycloalkyl ring. The heterocycloalkyl group can be substituted or unsubstituted.

The term “heterocycloalkylene” or “heterocycloalkylenyl” as used herein refers to a bivalent saturated cycloalkyl radical (e.g.,

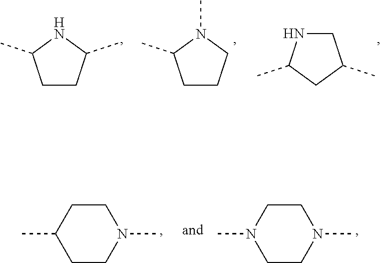

inter alia). In certain embodiments, the term may be regarded as a product of removal of two hydrogen atoms from the corresponding heterocycloalkane (e.g., piperidine) by removal of two hydrogen atoms from the same (e.g.

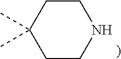

different (e.g.,

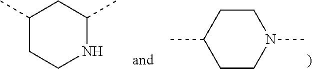

carbon atom(s) and/or heteroatom(s).

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl). 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylene” or “heteroarylenyl” as used herein refers to a bivalent heteroaryl radical (e.g., 2,4-pyridylene). In certain embodiments, the term may be regarded as a divalent radical formed by the removal of two hydrogen atoms from one or more rings of a heteroaryl moiety, wherein the hydrogen atoms may be removed from the same or different rings, preferably the same ring.

“Heteroarylalkyl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}—R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}—R_{f} where R_{b} is an alkynylene chain as defined above and R_{f} is a heteroaryl radical as defined above. Unless stated otherwise specifically in the specification, a heteroarylalkynyl group can be optionally substituted.

As used herein, the term “substituted” means any of the above groups (i.e., alkyl, alkylene, alkenyl, alkynyl, alkoxy, aryl, carbocyclyl, cycloalkyl, cycloalkenyl, cycloalkynyl, heterocyclyl, and/or heteroaryl) wherein at least hydrogen atom is replaced by a bond to a non-hydrogen atom or group of atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. The term “substituted” further refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In yet other embodiments, the substituents vary in number between one and three. In yet another embodiment, the substituents vary in number between one and two. “Substituted” also means any of the above groups in which one or more hydrogen atoms are replaced by a higher-order bond (e.g., a double- or triple-bond) to a heteroatom such as oxygen in oxo, carbonyl, carboxyl, carboxylic acid and ester groups; and nitrogen in groups such as imines, oximes, hydrazones, and nitriles. For example, “substituted” includes any of the above groups in which one or more hydrogen atoms are replaced with, for example, —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, =NSO₂R_{g}, and —SO₂NR_{g}Rₕ. “Substituted” also means any of the above groups in which one or more hydrogen atoms are replaced with, for example, —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently selected from any of the above groups, including but not limited to: hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to any of the above groups, including but not limited to amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group.

In addition, each of the foregoing substituents can also be optionally substituted with one or more of the above substituents.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “hydroxy” or “hydroxyl” refers to a group of the formula —OH group.

“Immunogen” refers to any substance introduced into the body in order to generate an immune response. That substance can a physical molecule, such as a protein, or can be encoded by a vector, such as DNA, mRNA, or a virus.

The term “imino” refers to a group of the formula=NH group.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “linker” as used herein refers to an organic moiety that connects two parts of a compound (e.g., a small molecule drug and an antibody). The linker can be, in non-limiting examples, a direct bond, a single atom (e.g., —O—), a peptide, or a substituted or unsubstituted alkylene or heteroalkylene moiety (e.g., polyethylene glycol). One skilled in the art would be apprised of the common linkers suitable for use in antibody drug conjugates and methods of preparation thereof.

The term “isomers” or “stereoisomers” refers to compounds, which have identical chemical constitution, but differ with regard to the arrangement of the atoms or groups in space.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The term “nitro” refers to a group of the formula —NO₂ group.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

As used herein, the term “optionally substituted” means that the referenced group may be substituted or unsubstituted. In certain embodiments, the referenced group is optionally substituted with zero substituents, i.e., the referenced group is unsubstituted. In yet other embodiments, the referenced group is optionally substituted with one or more additional group(s) individually and independently selected from groups described herein.

The term “oxo” refers to a group of the formula the ═O group.

“Parenteral” administration of a composition includes, e.g., subcutaneous (s.c.), intravenous (i.v.), intramuscular (i.m.), or intrasternal injection, or infusion techniques.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “pharmacological composition,” “therapeutic composition,” “therapeutic formulation” or “pharmaceutically acceptable formulation” can mean, but is in no way limited to, a composition or formulation that allows for the effective distribution of an agent provided by the disclosure, which is in a form suitable for administration to the physical location most suitable for their desired activity, e.g., systemic administration. The pharmaceutical composition facilitates administration of the compound to an organism. Multiple techniques of administering a compound exist in the art including, but not limited to, intravenous, oral, aerosol, parenteral, ophthalmic, pulmonary and topical administration.

Non-limiting examples of agents suitable for formulation with the, e.g., compounds provided by the instant disclosure include: cinnamoyl, PEG, phospholipids or lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The term “pharmaceutically acceptable” or “pharmacologically acceptable” can mean, but is in no way limited to, entities and compositions that do not produce an adverse, allergic or other untoward reaction when administered to an animal, or a human, as appropriate.

The term “pharmaceutically acceptable carrier” or “pharmacologically acceptable carrier” can mean, but is in no way limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In certain instances, the polynucleotide or nucleic acid of the disclosure is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27: 196-197).

As used herein, the term “prodrug” refers to an agent that is converted into the parent drug in vivo. For example, the term “prodrug” refers to a derivative of a known direct acting drug, which derivative has enhanced delivery characteristics and therapeutic value as compared to the drug, and is transformed into the active drug by an enzymatic or chemical process. In some embodiments, “prodrug” refers to an inactive or relatively less active form of an active agent that becomes active by undergoing a chemical conversion through one or more metabolic processes. In certain embodiments, upon in vivo administration, a prodrug is chemically converted to the biologically, pharmaceutically, or therapeutically active form of the compound. In yet other embodiments, a prodrug is enzymatically metabolized by one or more steps or processes to the biologically, pharmaceutically, or therapeutically active form of the compound. For example, the present compounds can be administered to a subject as a prodrug that includes an initiator bound to an active agent, and, by virtue of being degraded by a metabolic process, the active agent is released in its active form.

The term “promoter” as used herein is defined as a DNA sequence recognized by the synthetic machinery of the cell, or introduced synthetic machinery, required to initiate the specific transcription of a polynucleotide sequence. For example, the promoter that is recognized by bacteriophage RNA polymerase and is used to generate the mRNA by in vitro transcription.

In certain embodiments, “pseudouridine” refers, in yet other embodiments, to m¹acp³Y (1-methyl-3-(3-amino-3-carboxypropyl) pseudouridine. In yet other embodiments, the term refers to m¹Y (1-methylpseudouridine). In yet other embodiments, the term refers to Ym (2′-O-methylpseudouridine. In yet other embodiments, the term refers to m⁵D (5-methyldihydrouridine). In yet other embodiments, the term refers to m³Y (3-methylpseudouridine). In yet other embodiments, the term refers to a pseudouridine moiety that is not further modified. In yet other embodiments, the term refers to a monophosphate, diphosphate, or triphosphate of any of the above pseudouridines. In yet other embodiments, the term refers to any other pseudouridine known in the art. Each possibility represents a separate embodiment of the present disclosure.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

The term “tautomers” are constitutional isomers of organic compounds that readily interconvert by a chemical process (tautomerization).

The term “therapeutic” as used herein means a treatment and/or prophylaxis. A therapeutic effect is obtained by suppression, diminution, remission, or eradication of at least one sign or symptom of a disease or disorder state.

As used herein, the terms “therapeutic compound”, “therapeutic agent”. “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, hydrophilic therapeutic agents, hydrophobic therapeutic agents, antibiotics, antibodies, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium or high melting point. The therapeutic agents may be provided with or without a stabilizing salt or salts.

Some examples of active ingredients suitable for use in the pharmaceutical formulations and methods of the present disclosure include: hydrophilic, lipophilic, amphiphilic or hydrophobic, and that can be solubilized, dispersed, or partially solubilized and dispersed, on or about the nanocluster. The active agent-nanocluster combination may be coated further to encapsulate the agent-nanocluster combination and may be directed to a target by functionalizing the nanocluster with, e.g., aptamers and/or antibodies. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional agents, and the like. The active agents described herein may be found in their native state, however, they will generally be provided in the form of a salt. The active agents described herein include their isomers, analogs and derivatives.

“Thioalkyl” refers to a formula —SRₐ where Rₐ is an alkyl, alkenyl, or alkynyl as defined above containing 1 to 20 carbon atoms. Unless stated otherwise specifically in the specification, a thioalkyl group can be optionally substituted.

The term “thioxo” refers to a group of the formula the ═S group.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

As used herein, “treating a disease or disorder” means reducing the frequency with which a symptom of the disease or disorder is experienced by a patient. Disease and disorder are used interchangeably herein.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, and so forth, as well as individual numbers within that range, for example, 1, 2, 2.7, 3, 4, 5, 5.3, and 6. This applies regardless of the breadth of the range.

### Biodegradable Lipidic Compounds

In one aspect, the present disclosure provides a compound of Formula (1-1), or a salt, solvate, stereoisomer, isotopologue, or derivative thereof:

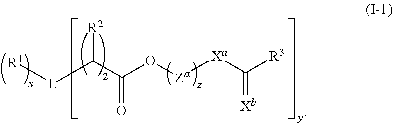

In another aspect, the present disclosure provides a compound of Formula (I), or a salt, solvate, stereoisomer, isotopologue, or derivative thereof:

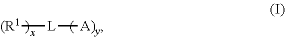

wherein:
   each occurrence of A is independently

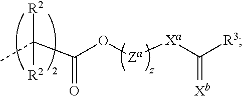

L is an amine linker selected from the group consisting of optionally substituted aminoalkyl linker, optionally substituted diaminoalkyl linker, optionally substituted triaminoalkyl linker, optionally substituted tetraaminoalkyl linker, optionally substituted pentaaminoalkyl linker, optionally substituted polyaminoalkyl linker, optionally substituted aminocycloalkyl linker, optionally substituted diaminocycloalkyl linker, optionally substituted triaminocycloalkyl linker, optionally substituted tetraaminocycloalkyl linker, optionally substituted pentaaminocycloalkyl linker, and optionally substituted polyaminocycloalkyl linker;
each occurrence of X^{a} and X^{b} is independently selected from the group consisting of —O—, —S—, —N(R⁶)_{y′}—, —P(R⁶)_{y′}—;
each occurrence of Z^{a} is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, and optionally substituted C₂-C₈ heterocyloalkylenyl;
each occurrence of R¹, R², and R³, if present, is independently selected from the group consisting of hydrogen, halogen, optionally substituted C₁-C₁₂ alkyl, optionally substituted C₃-C₁₂ cycloalkyl, optionally substituted —Y(R⁶)_{z′}(R⁷)_{z″}—(C₃-C₁₂ cycloalkyl), optionally substituted C₂-C₁₂ heterocycloalkyl, optionally substituted-(R⁶)_{z′}(R⁷)_{z″}—(C₂-C₁₂ heterocycloalkyl), optionally substituted C₂-C₁₂ alkenyl, optionally substituted C₅-C₁₂ cycloalkenyl, optionally substituted —Y(R⁶)_{z′}(R⁷)_{z″}—(C₅-C₁₂ cycloalkenyl), optionally substituted C₂-C₁₂ alkynyl, optionally substituted C₈-C₁₂ cycloalkynyl, optionally substituted —Y(R⁶)_{z′}(R⁷)_{z″}—(C₅-C₁₂ cycloalkynyl), optionally substituted C₆-C₁₀ aryl, optionally substituted —Y(R⁶)_{z′}(R⁷)_{z″}—(C₆-C₁₀ aryl), optionally substituted C₂-C₁₂ heteroaryl, optionally substituted —Y(R⁶)_{z′}(R⁷)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁶)_{z′}(R⁷)_{z″}-ester, —Y(R⁶)_{z′}(R⁷)_{z″}, —NO₂, —CN, sulfoxy, sulfonate, sulfate, sulfite, and sulfide, or two geminal R² groups can combine to form ═O or ═S;
each occurrence of R⁶ and R⁷ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, ═S, sulfoxy, sulfonate, sulfate, sulfite, and sulfide, or two geminal R⁶ and R⁷ groups can combine to form ═O or ═S;
each occurrence of Y is independently selected from the group consisting of C, O, N, S, P, and Si; and
x is an integer from 0 to 10;
y is an integer from 1 to 10;
each occurrence of z is an integer from 0 to 20;
each occurrence of y′, z′ and z″ is independently an integer represented by 0, 1, or 2.

In certain embodiments, L comprises 1 instance of N. In certain embodiments, L comprises 2 instances of N. In certain embodiments, L comprises 3 instances of N. In certain embodiments, L comprises 4 instances of N. In certain embodiments, L comprises 5 instances of N.

In certain embodiments, each N atom in L is independently substituted with 0, 1, or-2 instances of A.

In certain embodiments, x is 0. In certain embodiments, x is 1. In certain embodiments, x is 2. In certain embodiments, x is 3. In certain embodiments, x is 4. In certain embodiments, x is 5. In certain embodiments, x is 6. In certain embodiments, x is 7. In certain embodiments, x is 8. In certain embodiments, x is 9. In certain embodiments, x is 10.

In certain embodiments, L is selected from the group consisting of

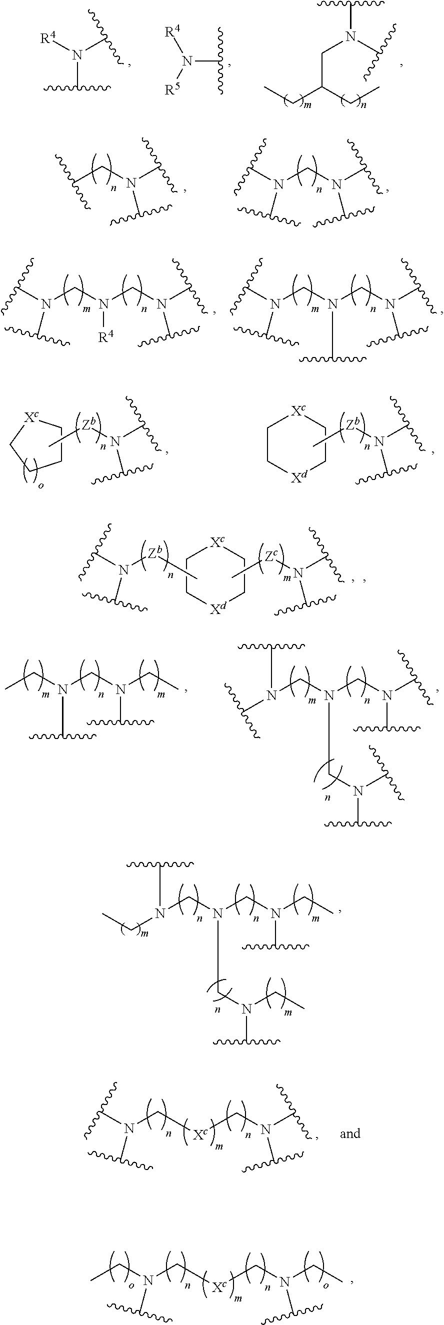

wherein:
   R⁴ and R⁵, if present, are each independently selected from the group consisting of hydrogen, halogen, optionally substituted C₁-C₁₂ alkyl, optionally substituted C₃-C₁₂ cycloalkyl, optionally substituted —Y(R⁶)_{z′}(R⁷)_{z″}—(C₃-C₁₂ cycloalkyl), optionally substituted C₂-C₁₂ heterocycloalkyl, optionally substituted-(R⁶)_{z′}(R⁷)_{z″}—(C₂-C₁₂ heterocycloalkyl), optionally substituted C₂-C₁₂ alkenyl, optionally substituted C₅-C₁₂ cycloalkenyl, optionally substituted —Y(R⁶)_{z′}(R⁷)_{z″}—(C₅-C₁₂ cycloalkenyl), optionally substituted C₂-C₁₂ alkynyl, optionally substituted C₅-C₁₂ cycloalkynyl, optionally substituted —Y(R⁶)_{z′}(R⁷)_{z″}—(C₅-C₁₂ cycloalkynyl), optionally substituted C₆-C₁₀ aryl, optionally substituted —Y(R⁶)_{z′}(R⁷)_{z″}—(C₆-C₁₀ aryl), optionally substituted C₂-C₁₂ heteroaryl, optionally substituted —Y(R⁶)_{z′}—(R⁷)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁶)_{z′}(R⁷)_{z″}-ester, —Y(R⁶)_{z′}(R⁷)_{z′}, —NO₂, —CN, sulfoxy, sulfonate, sulfate, sulfite, and sulfide;
   each occurrence of X^{c} and X^{d} is independently selected from the group consisting of —O—, —S—, —N(R⁶)_{y′}—P(R⁶)_{y′}—;
   each occurrence of Z^{b} and Z^{c} is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, and optionally substituted C₂-C₈ heterocyloalkylenyl;
   each occurrence of m, n, and o is independently an integer from 0 to 10; and
   each occurrence of

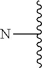 [SMILES: *N]

indicates a bond between a N atom and A or R¹, if present.

In certain embodiments, L is

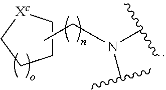

In certain embodiments, L is

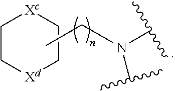

In certain embodiments, L is selected from the group consisting of

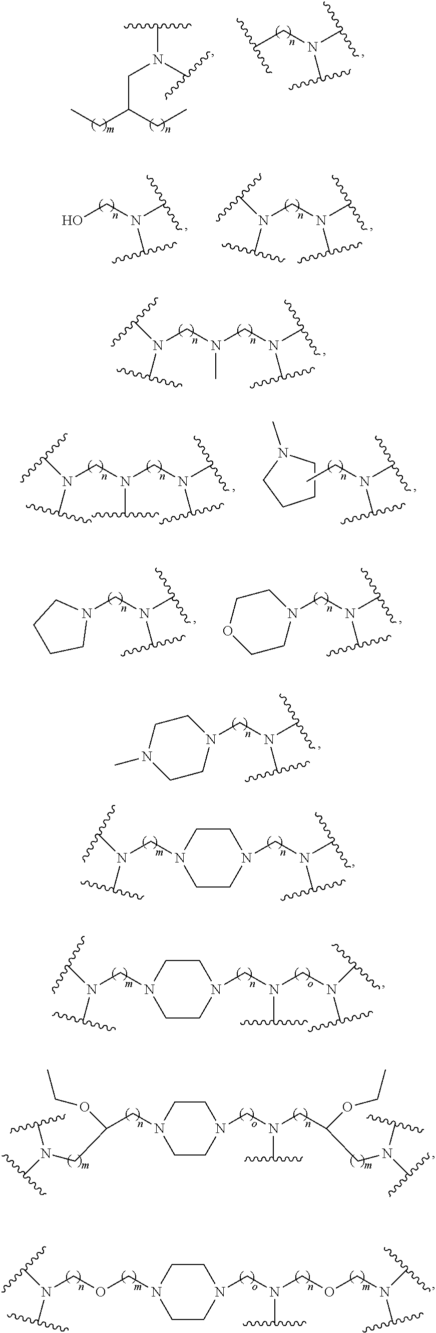

wherein:
   each occurrence of m, n, and o is independently an integer from 0 to 10; and
   each occurrence of

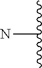 [SMILES: *N]

indicates a bond between a N atom and A or R¹, if present.

In certain embodiments, the compound of Formula (I) is a compound of Formula (II):

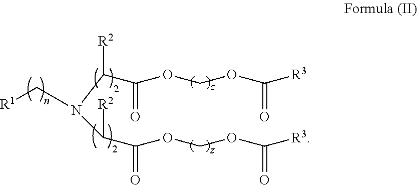

In certain embodiments, the compound of Formula (I) is a compound of Formula (III):

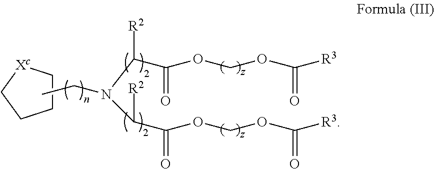

In certain embodiments, the compound of Formula (I) is a compound of Formula (IV):

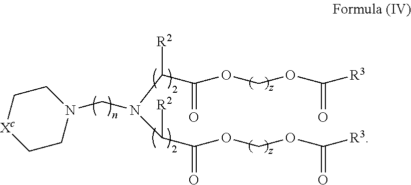

In certain embodiments, the compound of Formula (I) is a compound of Formula (V):

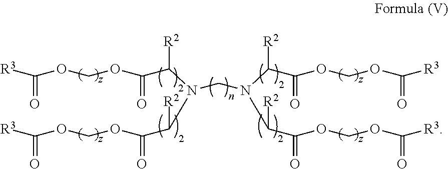

In certain embodiments, the compound of Formula (I) is a compound of Formula (VI):

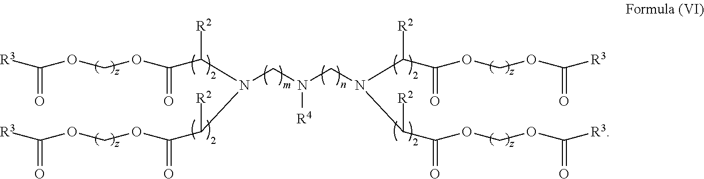

In certain embodiments, the compound of Formula (I) is a compound of Formula (VII):

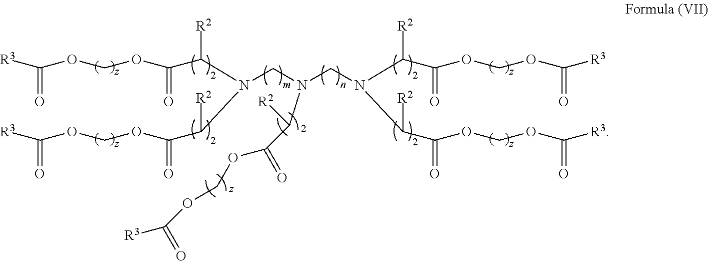

In certain embodiments, the compound of Formula (I) is a compound of Formula (VIII):

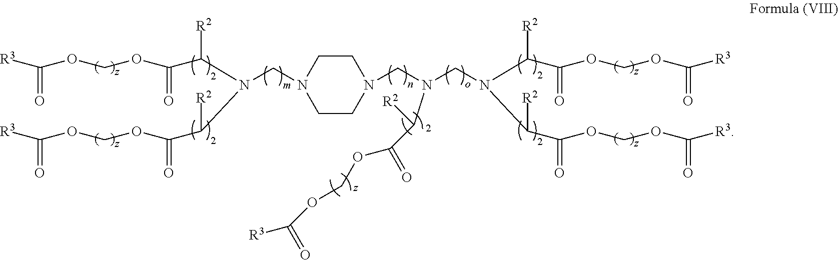

In certain embodiments, the compound of Formula (I) is a compound of Formula (IX):

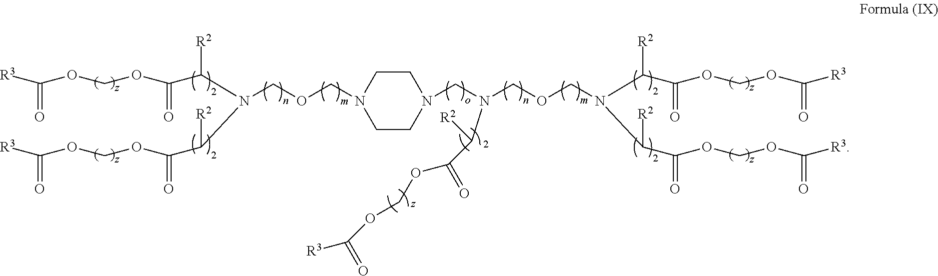

In certain embodiments, the compound of Formula (I) is a compound of Formula (X):

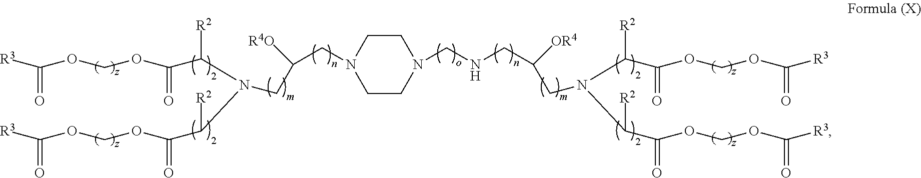

wherein:
   each occurrence of X^{c} is independently selected from the group consisting of —O—, —S—, —N(R⁶)_{y′}—, and —P(R⁶)_{y′}—;
   each occurrence of R⁴ is independently selected from the group consisting of hydrogen, halogen, optionally substituted C₁-C₁₂ alkyl, optionally substituted C₃-C₁₂ cycloalkyl, optionally substituted —Y(R⁶)_{z′}(R⁷)_{z″}—(C₃-C₁₂ cycloalkyl), optionally substituted C₂-C₁₂ heterocycloalkyl, optionally substituted-(R⁶)_{z′}(R⁷)_{z″}—(C₂-C₁₂ heterocycloalkyl), optionally substituted C₂-C₁₂ alkenyl, optionally substituted C₅-C₂ cycloalkenyl, optionally substituted —Y(R⁶)_{z′}(R⁷)_{z″}—(C₅-C₁₂ cycloalkenyl), optionally substituted C₂-C₁₂ alkynyl, optionally substituted C₅-C₁₂ cycloalkynyl, optionally substituted —Y(R⁶)_{z′}(R⁷)_{z″}—(C₈-C₁₂ cycloalkynyl), optionally substituted C₆-C₁₀ aryl, optionally substituted —Y(R⁶)_{z′}(R⁷)_{z″}—(C₆-C₁₀ aryl), optionally substituted C₂-C₁₂ heteroaryl, optionally substituted —Y(R⁶)_{z′}(R⁷)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate. acyl, hydroxyl, hydroxyalkyl, hydroxyalkenyl, hydroxyalkynyl, hydroxyaryl, alkoxy, carboxyl, carboxylate, ester, —Y(R⁶)_{z′}(R⁷)_{z″}-ester, —Y(R⁶)_{z′}(R⁷)_{z″}, —NO₂, —CN, sulfoxy, sulfonate, sulfate, sulfite, and sulfide; and
   each occurrence of m, n, and o is independently an integer from 0 to 10.

In some embodiments, each occurrence of X^{a}, X^{b}, X^{c}, and X^{d} is independently selected from O, S, N(R⁶)_{y′}, P(R⁶)_{y′}, or any combination thereof.

In some embodiments, each occurrence of Z^{a}, Z^{b}, and Z° is independently selected from alkyl, substituted alkyl, cycloalkyl, substituted cycloalkyl, —Y(R⁶)_{z′}(R⁷)_{z″}-cycloalkyl, substituted —Y(R⁶)_{z′}(R⁷)_{z″}-cycloalkyl, heterocycloalkyl, substituted heterocycloalkyl, —Y(R⁶)_{z′}(R⁷)_{z″}-heterocycloalkyl, substituted-(R⁶)_{z′}(R⁷)_{z″}-heterocycloalkyl, alkenyl, substituted alkenyl, cycloalkenyl, substituted cycloalkenyl, —Y(R⁶)_{z′}(R⁷)_{z″}-cycloalkenyl, substituted —Y(R⁶)_{z′}(R⁷)_{z″}-cycloalkenyl, alkynyl, substituted alkynyl, cycloalkynyl, substituted cycloalkynyl, —Y(R⁶)_{z′}(R⁷)_{z″}-cycloalkynyl, substituted —Y(R⁶)_{z′}(R⁷)_{z″}-cycloalkynyl, aryl, substituted aryl, —Y(R⁶)_{z′}(R⁷)_{z″}-aryl, substituted —Y(R⁶)_{z′}(R⁷)_{z″}-aryl, heteroaryl, substituted heteroaryl, —Y(R⁶)_{z′}(R⁷)_{z″}-heteroaryl, substituted —Y(R⁶)_{z′}(R⁷)_{z″}-heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁶)_{z′}(R⁷)_{z″}-ester, —Y(R⁶)_{z′}(R⁷)_{z″}, ═O, —NO₂, —CN, ═S, sulfoxy, sulfonate, sulfate, sulfite, sulfide, or any combination thereof.

In some embodiments, each occurrence of R¹, R², R³, R⁴, and R⁵ is independently selected from hydrogen, halogen, alkyl, substituted alkyl, cycloalkyl, substituted cycloalkyl, —Y(R⁶)_{z′}(R⁷)_{z″}-cycloalkyl, substituted —Y(R⁶)_{z′}(R⁷)_{z″}-cycloalkyl, heterocycloalkyl, substituted heterocycloalkyl, —Y(R⁶)_{z′}(R⁷)_{z″}-heterocycloalkyl, substituted-(R⁶)_{z′}(R⁷)_{z″}-heterocycloalkyl, alkenyl, substituted alkenyl, cycloalkenyl, substituted cycloalkenyl, —Y(R⁶)_{z′}(R⁷)_{z′}-cycloalkenyl, substituted —Y(R⁶)_{z′}(R⁷)_{z″}-cycloalkenyl, alkynyl, substituted alkynyl, cycloalkynyl, substituted cycloalkynyl, —Y(R⁶)_{z′}(R⁷)_{z″}-cycloalkynyl, substituted —Y(R⁶)_{z′}(R⁷)_{z″}-cycloalkynyl, aryl, substituted aryl, —Y(R⁶)_{z′}(R⁷)_{z″}-aryl, substituted —Y(R⁶)_{z′}(R⁷)_{z″}-aryl, heteroaryl, substituted heteroaryl, —Y(R⁶)_{z′}(R⁷)_{z″}-heteroaryl, substituted —Y(R⁶)_{z′}(R⁷)_{z″}-heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁶)_{z′}(R⁷)_{z″}-ester, —Y(R⁶)_{z′}(R⁷)_{z″}, ═O, —NO₂, —CN, ═S, sulfoxy, sulfonate, sulfate, sulfite, sulfide, or any combination thereof.

For example, in some embodiments, each occurrence of R¹, R², R³, R⁴, and R⁵ is independently selected from hydrogen, alkyl, or substituted alkyl. In various embodiments, alkyl is C₁₋₂₀ alkyl. In certain embodiments, alkyl is C₁ alkyl. In certain embodiments, alkyl is C₂ alkyl. In certain embodiments, alkyl is C₃ alkyl. In certain embodiments, alkyl is C₄ alkyl. In certain embodiments, alkyl is C₅ alkyl. In certain embodiments, alkyl is C₆ alkyl. In certain embodiments, alkyl is C₇ alkyl. In certain embodiments, alkyl is C₈ alkyl. In certain embodiments, alkyl is C₉ alkyl. In certain embodiments, alkyl is C₁₀ alkyl. In certain embodiments, alkyl is C₁₁ alkyl. In certain embodiments, alkyl is C₁₂ alkyl. In certain embodiments, alkyl is C₁₃ alkyl. In certain embodiments, alkyl is C₁₄ alkyl. In certain embodiments, alkyl is C₁₅ alkyl. In certain embodiments, alkyl is C₁₆ alkyl. In certain embodiments, alkyl is C₁₇ alkyl. In certain embodiments, alkyl is C₁₈ alkyl. In certain embodiments, alkyl is C₁₉ alkyl. In certain embodiments, alkyl is C₂₀ alkyl.

In other embodiments, each occurrence of R¹, R², R³, R⁴, and R⁵ is branched alkyl. In various embodiments, branched alkyl is C₁₋₂₀ branched alkyl. In certain embodiments, branched alkyl is C₁ branched alkyl. In certain embodiments, branched alkyl is C₂ branched alkyl. In certain embodiments, branched alkyl is C₃ branched alkyl. In certain embodiments, branched alkyl is C₄ branched alkyl. In certain embodiments, branched alkyl is C₈ branched alkyl. In certain embodiments, branched alkyl is C₆ branched alkyl. In certain embodiments, branched alkyl is C₇ branched alkyl. In certain embodiments, branched alkyl is C₈ branched alkyl. In certain embodiments, branched alkyl is C₉ branched alkyl. In certain embodiments, branched alkyl is C₁₀ branched alkyl. In certain embodiments, branched alkyl is C₁₁ branched alkyl. In certain embodiments, branched alkyl is C₁₂ branched alkyl. In certain embodiments, branched alkyl is C₁₃ branched alkyl. In certain embodiments, branched alkyl is C₁₄ branched alkyl. In certain embodiments, branched alkyl is Cis branched alkyl. In certain embodiments, branched alkyl is C₁₆ branched alkyl. In certain embodiments, branched alkyl is C₁₇ branched alkyl. In certain embodiments, branched alkyl is Cis branched alkyl. In certain embodiments, branched alkyl is C₁₉ branched alkyl. In certain embodiments, branched alkyl is C₂₀ branched alkyl.

In some embodiments, each occurrence of Y is independently selected from C, O, N, S, P, or Si. For example, in certain embodiments, Y is C. In certain embodiments, Y is O. In certain embodiments, Y is N. In certain embodiments, Y is S.

In some embodiments, each occurrence of R⁶ and R⁷ is independently selected from hydrogen, halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, ═S, sulfoxy, sulfonate, sulfate, sulfite, sulfide, or any combination thereof.

In some embodiments, each occurrence of z′ is independently an integer represented by 0, 1, 2, or 3. For example, in certain embodiments, z′ is 0. In certain embodiments, z′ is 1. In certain embodiments, z′ is 2. In certain embodiments, z′ is 3.

In some embodiments, each occurrence of z″ is independently an integer represented by 0, 1, 2, or 3. For example, in certain embodiments, z″ is 0. In certain embodiments, z″ is 1. In certain embodiments, z″ is 2. In certain embodiments, z″ is 3.

In various embodiments, x is an integer from 0 to 10. In some embodiments, x is an integer from 1 to 10. In some embodiments, x is an integer from 0 to 5. In some embodiments, x is an integer from 0 to 2. For example, in certain embodiments, x is 0. In certain embodiments, x is 1. In certain embodiments, x is 2. In certain embodiments, x is 3. In certain embodiments, x is 4. In certain embodiments, x is 5. In certain embodiments, x is 6. In certain embodiments, x is 7. In certain embodiments, x is 8. In certain embodiments, x is 9. In certain embodiments, x is 10.

In various embodiments, y is an integer from 0 to 10. In some embodiments, y is an integer from 1 to 10. In some embodiments, y is an integer from 1 to 5. In some embodiments, y is an integer from 1 to 2. For example, in certain embodiments, y is 0. In certain embodiments, y is 1. In certain embodiments, y is 2. In certain embodiments, y is 3. In certain embodiments, y is 4. In certain embodiments, y is 5. In certain embodiments, y is 6. In certain embodiments, y is 7. In certain embodiments, y is 8. In certain embodiments, y is 9. In certain embodiments, y is 10.

In various embodiments, each occurrence of z is independently an integer from 0 to 20. In some embodiments, each occurrence of z is independently an integer from 1 to 10. In some embodiments, each occurrence of z is independently an integer from 1 to 5. In some embodiments, each occurrence of z is independently an integer from 0 to 2. For example, in certain embodiments, z is 0. In certain embodiments, z is 1. In certain embodiments, z is 2. In certain embodiments, z is 3. In certain embodiments, z is 4. In certain embodiments, z is 5. In certain embodiments, z is 6. In certain embodiments, z is 7. In certain embodiments, z is 8. In certain embodiments, z is 9. In certain embodiments, z is 10. In certain embodiments, z is 11. In certain embodiments, z is 12. In certain embodiments, z is 13. In certain embodiments, z is 14. In certain embodiments, z is 15. In certain embodiments, z is 16. In certain embodiments, z is 17. In certain embodiments, z is 18. In certain embodiments, z is 19. In certain embodiments, z is 20.

In various embodiments, each occurrence of m is independently an integer from 0 to 10. In some embodiments, each occurrence of m is independently an integer from 1 to 10. In some embodiments, each occurrence of m is independently an integer from 1 to 5. In some embodiments, each occurrence of m is independently an integer from 1 to 2. For example, in certain embodiments, m is 0. In certain embodiments, m is 1. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10.

In various embodiments, each occurrence of n is independently an integer from 0 to 10. In some embodiments, each occurrence of n is independently an integer from 1 to 10. In some embodiments, each occurrence of n is independently an integer from 1 to 5. In some embodiments, each occurrence of n is independently an integer from 1 to 3. For example, in certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In various embodiments, each occurrence of o is independently an integer from 0 to 10. In some embodiments, each occurrence of o is independently an integer from 1 to 10. In some embodiments, each occurrence of o is independently an integer from 1 to 5. In some embodiments, each occurrence of 0 is independently an integer from 1 to 2. For example, in certain embodiments, o is 0. In certain embodiments, o is 1. In certain embodiments, o is 2. In certain embodiments, o is 3. In certain embodiments, o is 4. In certain embodiments, o is 5. In certain embodiments, o is 6. In certain embodiments, o is 7. In certain embodiments, o is 8. In certain embodiments, o is 9. In certain embodiments, o is 10.

In certain embodiments, each occurrence of R¹, R³, and R⁴ is alkyl or substituted alkyl. In certain embodiments, each occurrence of R² is hydrogen. In certain embodiments, each occurrence of X^{c} is O or N(R⁶)_{y′}, wherein R⁶ is alkyl and y′ is 1. In certain embodiments, each occurrence of m and o is 2. In certain embodiments, each occurrence of n is 2 or 3. In certain embodiments, each occurrence of z is 4.

In certain embodiments, each occurrence of R² is H. In certain embodiments, each occurrence of Z^{a} is —CH₂—. In certain embodiments, each occurrence of z is an integer represented by 4. In certain embodiments, each occurrence of X^{a} is O. In certain embodiments, each occurrence of X^{b} is O. In certain embodiments, x is an integer represented by 0. In certain embodiments, R¹ is absent. In certain embodiments, y is an integer represented by 2, 3, 4, or 5.

In certain embodiments, each occurrence of R³ is independently selected from the group consisting of n-pentyl, n-hexyl, n-heptyl, n-octyl, n-nonyl, 1-pentenyl, 1-heptenyl, 1-methylpentyl, 1-methylhexyl, 1-ethylpentyl, 1-methylheptyl, 2-ethylhexyl, and 3-methylheptyl, optionally wherein each occurrence of R³ is identical.

In certain embodiments, the compound of Formula (I) is

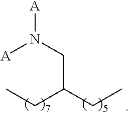

In certain embodiments, the compound of Formula (I) is

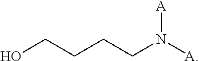

In certain embodiments, the compound of Formula (I) is

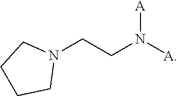

In certain embodiments, the compound of Formula (I) is

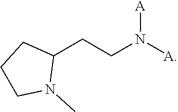

In certain embodiments, the compound of Formula (I) is

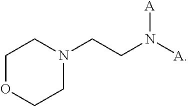

In certain embodiments, the compound of Formula (I) is

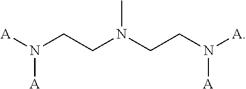

In certain embodiments, the compound of Formula (I) is

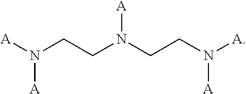

In certain embodiments, the compound of Formula (I) is

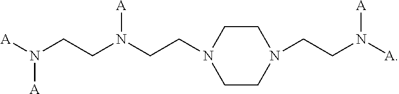

In certain embodiments, the compound of Formula (I) is

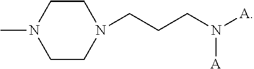

In certain embodiments, the compound of Formula (I) is

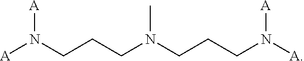

In certain embodiments, the compound of Formula (I) is

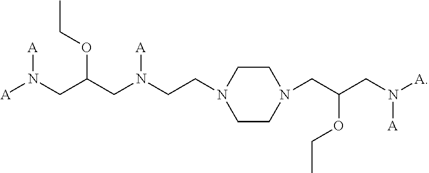

In certain embodiments, the compound of Formula (I) is

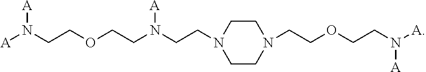

In certain embodiments, A is

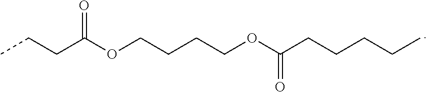

In certain embodiments, A is

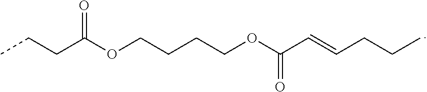

In certain embodiments, A is

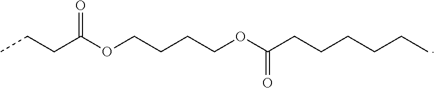

In certain embodiments, A is

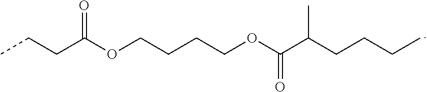

In certain embodiments, A is

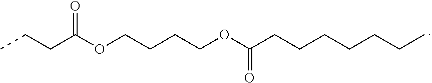

In certain embodiments, A is

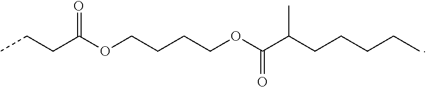

In certain embodiments, A is

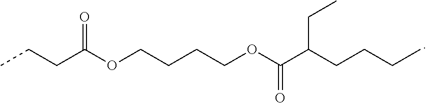

In certain embodiments, A is

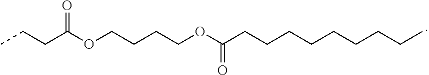

In certain embodiments, A is

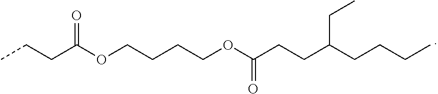

In certain embodiments, A is

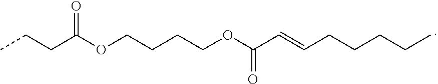

In certain embodiments, A is

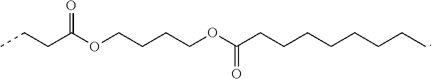

In certain embodiments, A is

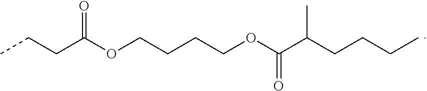

In certain embodiments, A is

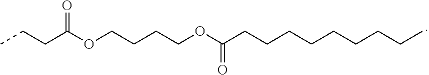

In certain embodiments, A is

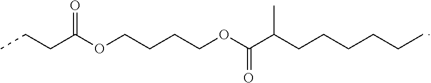

In certain embodiments, A is

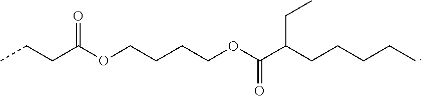

In certain embodiments, the compound of Formula (I) is

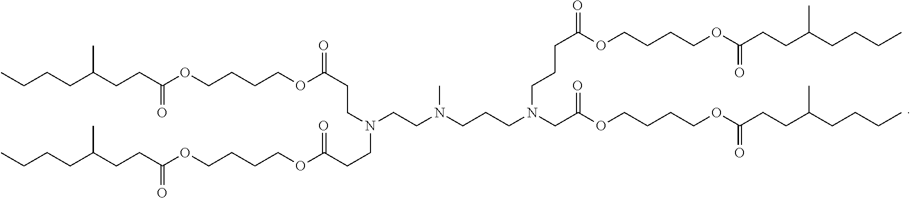 [SMILES: CCCCC(C)CCC(=O)OCCCCOC(=O)CCCN(CCCN(C)CCN(CCC(=O)OCCCCOC(=O)CCC(C)CCCC)CCC(=O)OCCCCOC(=O)CCC(C)CCCC)CC(=O)OCCCCOC(=O)CCC(C)CCCC]

In certain embodiments, the compound of Formula (I) is

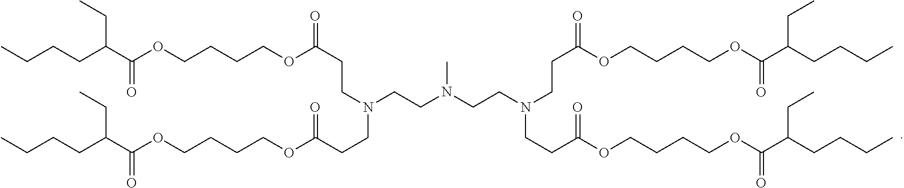 [SMILES: CCCCC(CC)C(=O)OCCCCOC(=O)CCN(CCC(=O)OCCCCOC(=O)C(CC)CCCC)CCN(C)CCN(CCC(=O)OCCCCOC(=O)C(CC)CCCC)CCC(=O)OCCCCOC(=O)C(CC)CCCC]

In certain embodiments, the compound of Formula (I) is

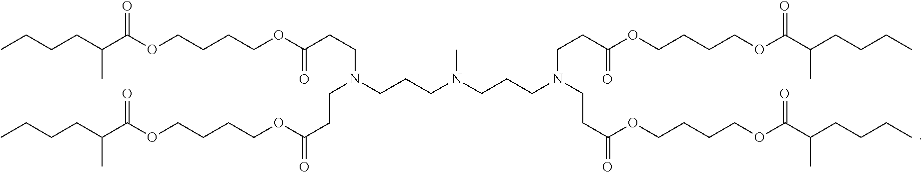 [SMILES: CCCCC(C)C(=O)OCCCCOC(=O)CCN(CCCN(C)CCCN(CCC(=O)OCCCCOC(=O)C(C)CCCC)CCC(=O)OCCCCOC(=O)C(C)CCCC)CCC(=O)OCCCCOC(=O)C(C)CCCC]

In certain embodiments, the compound of Formula (I) is

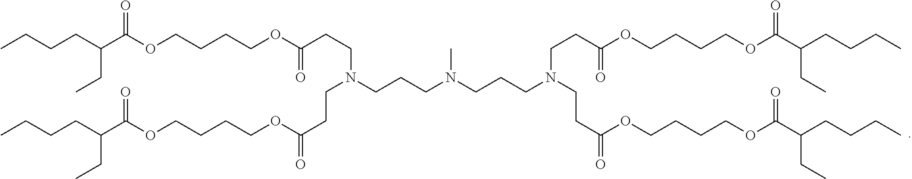 [SMILES: CCCCC(CC)C(=O)OCCCCOC(=O)CCN(CCCN(C)CCCN(CCC(=O)OCCCCOC(=O)C(CC)CCCC)CCC(=O)OCCCCOC(=O)C(CC)CCCC)CCC(=O)OCCCCOC(=O)C(CC)CCCC]

In certain embodiments, the compound of Formula (I) is

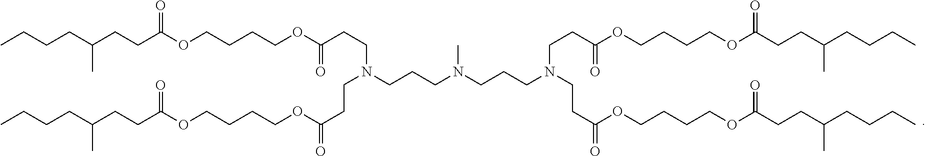 [SMILES: CCCCC(C)CCC(=O)OCCCCOC(=O)CCN(CCCN(C)CCCN(CCC(=O)OCCCCOC(=O)CCC(C)CCCC)CCC(=O)OCCCCOC(=O)CCC(C)CCCC)CCC(=O)OCCCCOC(=O)CCC(C)CCCC]

In certain embodiments, the compound of Formula (I) is

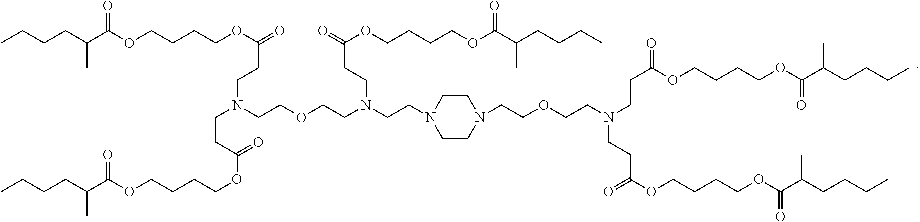 [SMILES: CCCCC(C)C(=O)OCCCCOC(=O)CCN(CCOCCN(CCC(=O)OCCCCOC(=O)C(C)CCCC)CCN1CCN(CCOCCN(CCC(=O)OCCCCOC(=O)C(C)CCCC)CCC(=O)OCCCCOC(=O)C(C)CCCC)CC1)CCC(=O)OCCCCOC(=O)C(C)CCCC]

In certain embodiments, the compound of Formula (I) is

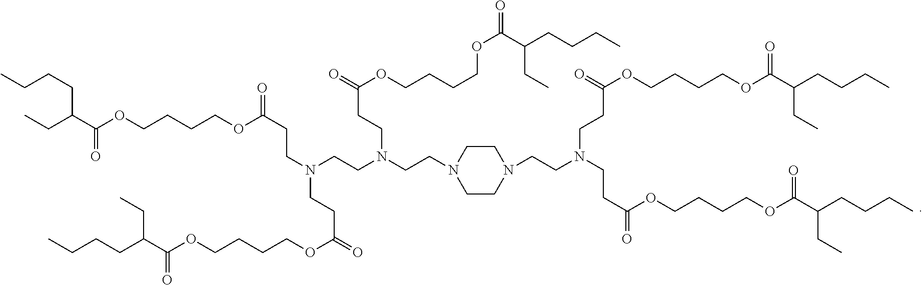 [SMILES: CCCCC(CC)C(=O)OCCCCOC(=O)CCN(CCC(=O)OCCCCOC(=O)C(CC)CCCC)CCN(CCC(=O)OCCCCOC(=O)C(CC)CCCC)CCN1CCN(CCN(CCC(=O)OCCCCOC(=O)C(CC)CCCC)CCC(=O)OCCCCOC(=O)C(CC)CCCC)CC1]

In certain embodiments, the compound of Formula (I) is

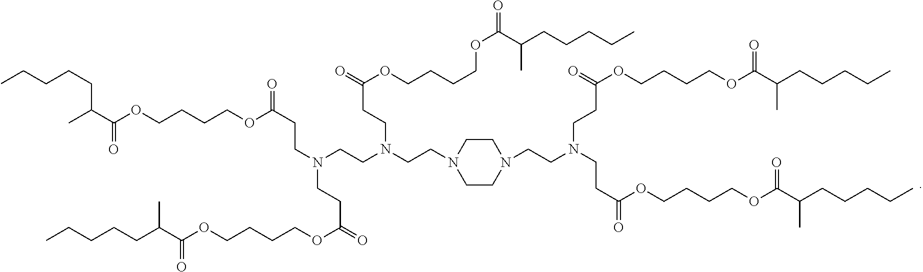 [SMILES: CCCCCC(C)C(=O)OCCCCOC(=O)CCN(CCC(=O)OCCCCOC(=O)C(C)CCCCC)CCN(CCC(=O)OCCCCOC(=O)C(C)CCCCC)CCN1CCN(CCN(CCC(=O)OCCCCOC(=O)C(C)CCCCC)CCC(=O)OCCCCOC(=O)C(C)CCCCC)CC1]

In certain embodiments, the compound of Formula (I) is

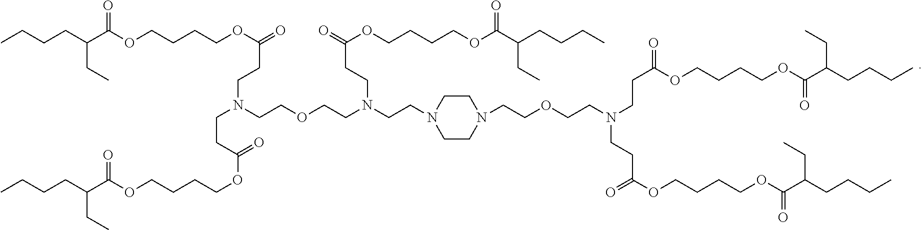 [SMILES: CCCCC(CC)C(=O)OCCCCOC(=O)CCN(CCOCCN(CCC(=O)OCCCCOC(=O)C(CC)CCCC)CCN1CCN(CCOCCN(CCC(=O)OCCCCOC(=O)C(CC)CCCC)CCC(=O)OCCCCOC(=O)C(CC)CCCC)CC1)CCC(=O)OCCCCOC(=O)C(CC)CCCC]

In certain embodiments, the compound of formula (I) is

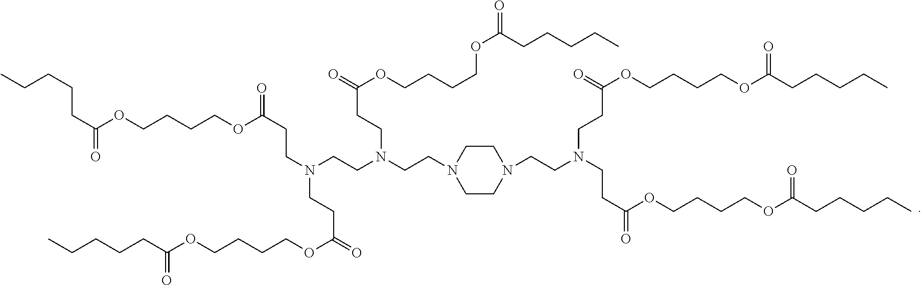 [SMILES: CCCCCC(=O)OCCCCOC(=O)CCN(CCC(=O)OCCCCOC(=O)CCCCC)CCN(CCC(=O)OCCCCOC(=O)CCCCC)CCN1CCN(CCN(CCC(=O)OCCCCOC(=O)CCCCC)CCC(=O)OCCCCOC(=O)CCCCC)CC1]

In certain embodiments, the compound of formula (I) is

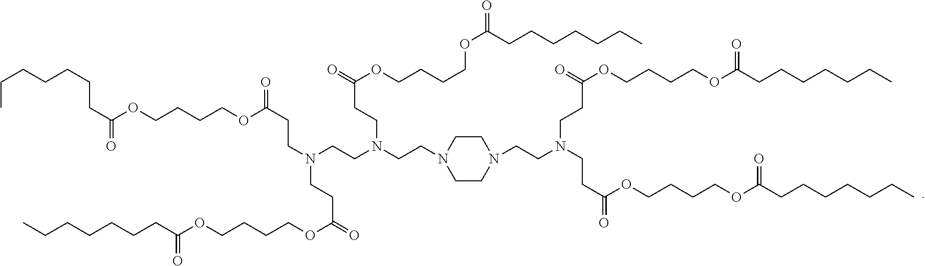 [SMILES: CCCCCCCC(=O)OCCCCOC(=O)CCN(CCC(=O)OCCCCOC(=O)CCCCCCC)CCN(CCC(=O)OCCCCOC(=O)CCCCCCC)CCN1CCN(CCN(CCC(=O)OCCCCOC(=O)CCCCCCC)CCC(=O)OCCCCOC(=O)CCCCCCC)CC1]

In certain embodiments, the compound of formula (I) is

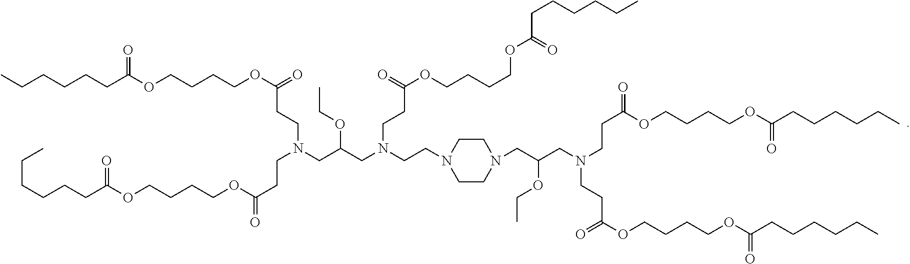 [SMILES: CCCCCCC(=O)OCCCCOC(=O)CCN(CCC(=O)OCCCCOC(=O)CCCCCC)CC(CN(CCC(=O)OCCCCOC(=O)CCCCCC)CCN1CCN(CC(CN(CCC(=O)OCCCCOC(=O)CCCCCC)CCC(=O)OCCCCOC(=O)CCCCCC)OCC)CC1)OCC]

In certain embodiments, the compound of formula (I) is

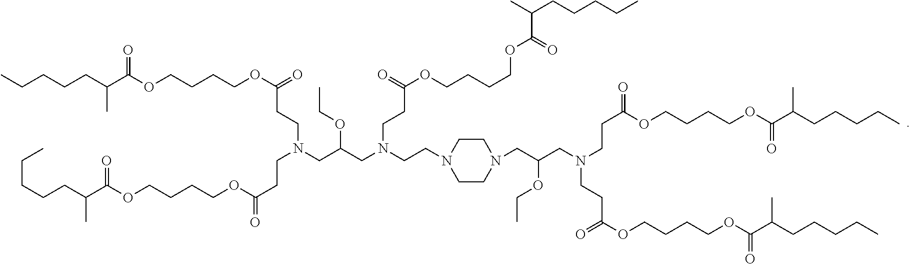 [SMILES: CCCCCC(C)C(=O)OCCCCOC(=O)CCN(CCC(=O)OCCCCOC(=O)C(C)CCCCC)CC(CN(CCC(=O)OCCCCOC(=O)C(C)CCCCC)CCN1CCN(CC(CN(CCC(=O)OCCCCOC(=O)C(C)CCCCC)CCC(=O)OCCCCOC(=O)C(C)CCCCC)OCC)CC1)OCC]

In certain embodiments, the compound of formula (I) is

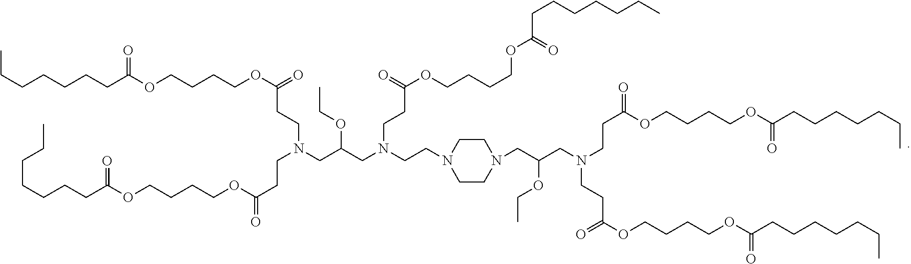 [SMILES: CCCCCCCC(=O)OCCCCOC(=O)CCN(CCC(=O)OCCCCOC(=O)CCCCCCC)CC(CN(CCC(=O)OCCCCOC(=O)CCCCCCC)CCN1CCN(CC(CN(CCC(=O)OCCCCOC(=O)CCCCCCC)CCC(=O)OCCCCOC(=O)CCCCCCC)OCC)CC1)OCC]

In certain embodiments, the compound of formula (I) is

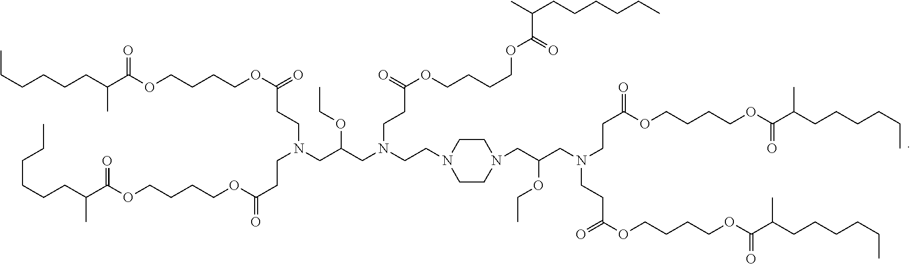 [SMILES: CCCCCCC(C)C(=O)OCCCCOC(=O)CCN(CCC(=O)OCCCCOC(=O)C(C)CCCCCC)CC(CN(CCC(=O)OCCCCOC(=O)C(C)CCCCCC)CCN1CCN(CC(CN(CCC(=O)OCCCCOC(=O)C(C)CCCCCC)CCC(=O)OCCCCOC(=O)C(C)CCCCCC)OCC)CC1)OCC]

In certain embodiments, the compound of formula (I) is

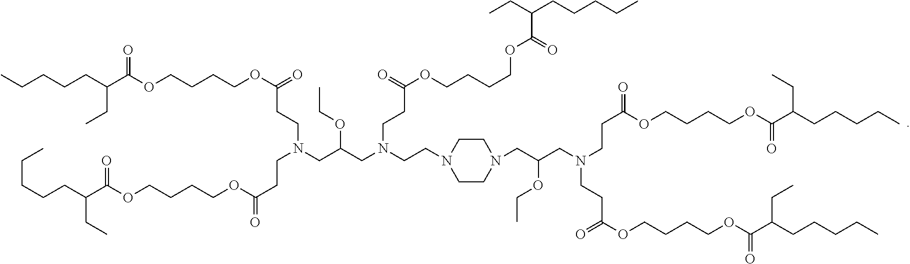 [SMILES: CCCCCC(CC)C(=O)OCCCCOC(=O)CCN(CCC(=O)OCCCCOC(=O)C(CC)CCCCC)CC(CN(CCC(=O)OCCCCOC(=O)C(CC)CCCCC)CCN1CCN(CC(CN(CCC(=O)OCCCCOC(=O)C(CC)CCCCC)CCC(=O)OCCCCOC(=O)C(CC)CCCCC)OCC)CC1)OCC]

In certain embodiments, the compound of formula (I) is

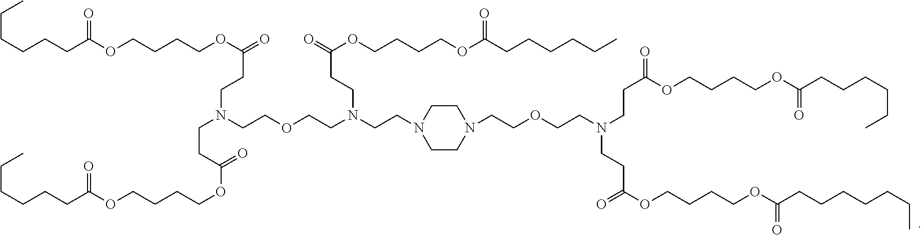 [SMILES: CCCCCCCC(=O)OCCCCOC(=O)CCN(CCOCCN1CCN(CCN(CCOCCN(CCC(=O)OCCCCOC(=O)CCCCCC)CCC(=O)OCCCCOC(=O)CCCCCC)CCC(=O)OCCCCOC(=O)CCCCCC)CC1)CCC(=O)OCCCCOC(=O)CCCCCC]

In certain embodiments, the compound of formula (I) is

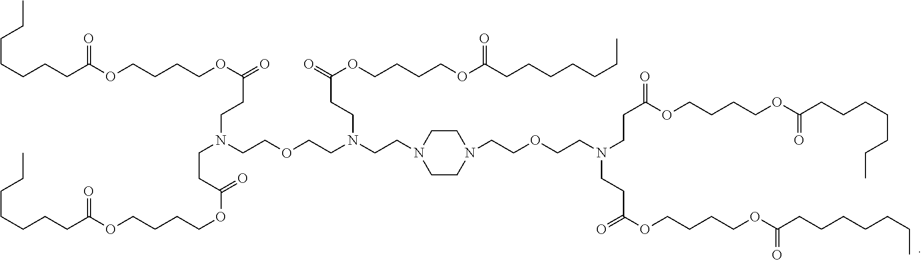 [SMILES: CCCCCCCC(=O)OCCCCOC(=O)CCN(CCOCCN(CCC(=O)OCCCCOC(=O)CCCCCCC)CCN1CCN(CCOCCN(CCC(=O)OCCCCOC(=O)CCCCCCC)CCC(=O)OCCCCOC(=O)CCCCCCC)CC1)CCC(=O)OCCCCOC(=O)CCCCCCC]

In certain embodiments, the compound of formula (I) is

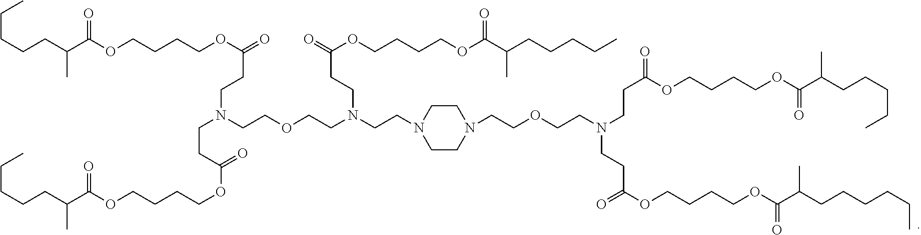 [SMILES: CCCCCCC(C)C(=O)OCCCCOC(=O)CCN(CCOCCN1CCN(CCN(CCOCCN(CCC(=O)OCCCCOC(=O)C(C)CCCCC)CCC(=O)OCCCCOC(=O)C(C)CCCCC)CCC(=O)OCCCCOC(=O)C(C)CCCCC)CC1)CCC(=O)OCCCCOC(=O)C(C)CCCCC]

In certain embodiments, the compound of formula (I) is

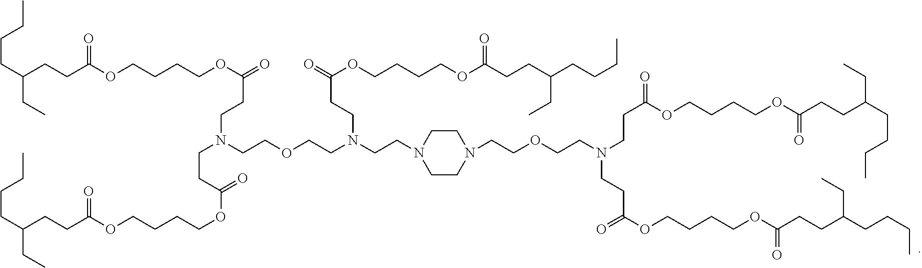 [SMILES: CCCCC(CC)CCC(=O)OCCCCOC(=O)CCN(CCOCCN(CCC(=O)OCCCCOC(=O)CCC(CC)CCCC)CCN1CCN(CCOCCN(CCC(=O)OCCCCOC(=O)CCC(CC)CCCC)CCC(=O)OCCCCOC(=O)CCC(CC)CCCC)CC1)CCC(=O)OCCCCOC(=O)CCC(CC)CCCC]

In certain embodiments, the compound of formula (I) is

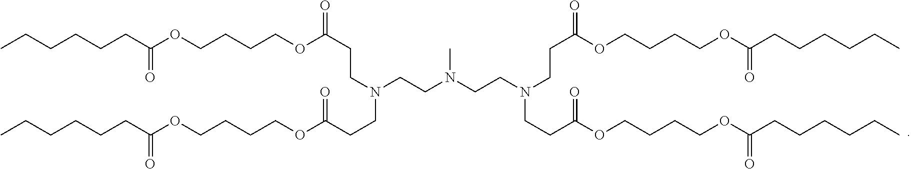 [SMILES: CCCCCCC(=O)OCCCCOC(=O)CCN(CCC(=O)OCCCCOC(=O)CCCCCC)CCN(C)CCN(CCC(=O)OCCCCOC(=O)CCCCCC)CCC(=O)OCCCCOC(=O)CCCCCC]

In certain embodiments, the compound of formula (I) is

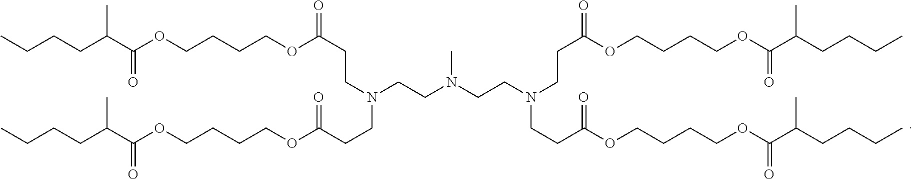 [SMILES: CCCCC(C)C(=O)OCCCCOC(=O)CCN(CCC(=O)OCCCCOC(=O)C(C)CCCC)CCN(C)CCN(CCC(=O)OCCCCOC(=O)C(C)CCCC)CCC(=O)OCCCCOC(=O)C(C)CCCC]

In certain embodiments, the compound of formula (I) is

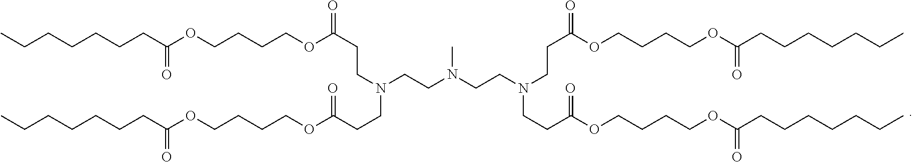 [SMILES: CCCCCCCC(=O)OCCCCOC(=O)CCN(CCC(=O)OCCCCOC(=O)CCCCCCC)CCN(C)CCN(CCC(=O)OCCCCOC(=O)CCCCCCC)CCC(=O)OCCCCOC(=O)CCCCCCC]

In certain embodiments, the compound of formula (I) is

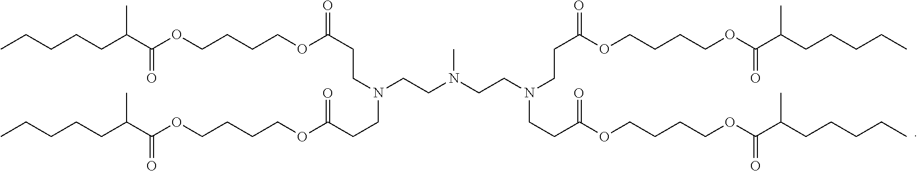 [SMILES: CCCCCC(C)C(=O)OCCCCOC(=O)CCN(CCC(=O)OCCCCOC(=O)C(C)CCCCC)CCN(C)CCN(CCC(=O)OCCCCOC(=O)C(C)CCCCC)CCC(=O)OCCCCOC(=O)C(C)CCCCC]

In certain embodiments, the compound of formula (I) is

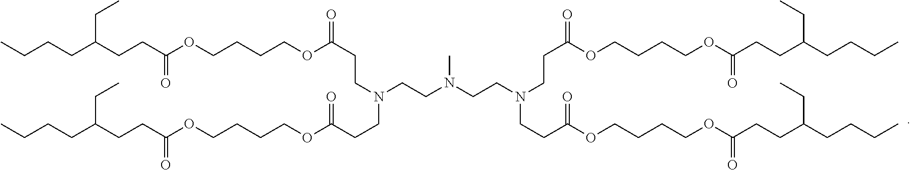 [SMILES: CCCCC(CC)CCC(=O)OCCCCOC(=O)CCN(CCC(=O)OCCCCOC(=O)CCC(CC)CCCC)CCN(C)CCN(CCC(=O)OCCCCOC(=O)CCC(CC)CCCC)CCC(=O)OCCCCOC(=O)CCC(CC)CCCC]

In certain embodiments, the compound of formula (I) is

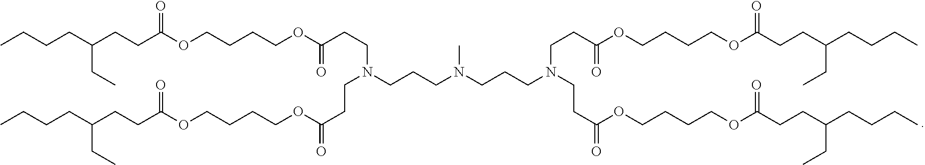 [SMILES: CCCCC(CC)CCC(=O)OCCCCOC(=O)CCN(CCCN(C)CCCN(CCC(=O)OCCCCOC(=O)CCC(CC)CCCC)CCC(=O)OCCCCOC(=O)CCC(CC)CCCC)CCC(=O)OCCCCOC(=O)CCC(CC)CCCC]

In certain embodiments, the compound of formula (I) is

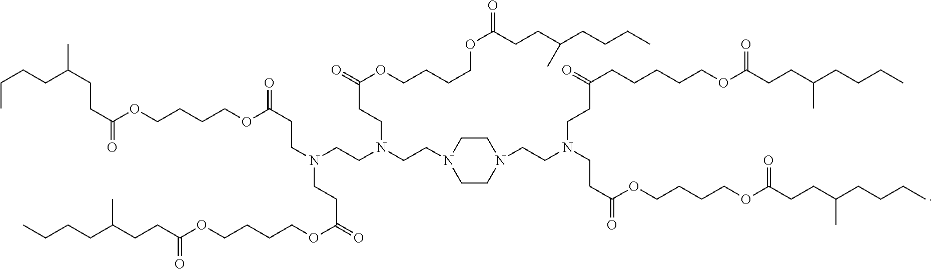 [SMILES: CCCCC(C)CCC(=O)OCCCCCC(=O)CCN(CCC(=O)OCCCCOC(=O)CCC(C)CCCC)CCN1CCN(CCN(CCC(=O)OCCCCOC(=O)CCC(C)CCCC)CCN(CCC(=O)OCCCCOC(=O)CCC(C)CCCC)CCC(=O)OCCCCOC(=O)CCC(C)CCCC)CC1]

In certain embodiments, the compound of formula (I) is

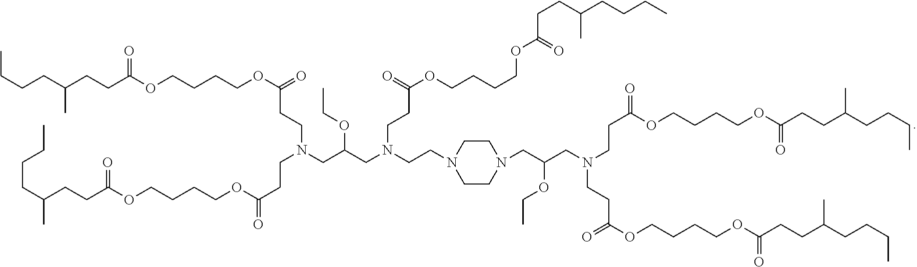 [SMILES: CCCCC(C)CCC(=O)OCCCCOC(=O)CCN(CCC(=O)OCCCCOC(=O)CCC(C)CCCC)CC(CN(CCC(=O)OCCCCOC(=O)CCC(C)CCCC)CCN1CCN(CC(CN(CCC(=O)OCCCCOC(=O)CCC(C)CCCC)CCC(=O)OCCCCOC(=O)CCC(C)CCCC)OCC)CC1)OCC]

In certain embodiments, the compound of formula (T) is

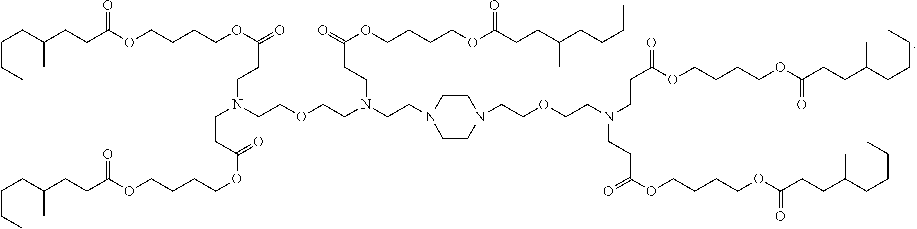 [SMILES: CCCCC(C)CCC(=O)OCCCCOC(=O)CCN(CCOCCN(CCC(=O)OCCCCOC(=O)CCC(C)CCCC)CCN1CCN(CCOCCN(CCC(=O)OCCCCOC(=O)CCC(C)CCCC)CCC(=O)OCCCCOC(=O)CCC(C)CCCC)CC1)CCC(=O)OCCCCOC(=O)CCC(C)CCCC]

In various embodiments, the compound having the structure of Formula (I) is a lipid. In various embodiments, the compound having the structure of Formula (I) is a lipidoid. In various embodiments, the compound having the structure of Formula (I) is an ionizable lipid. In various embodiments, the compound having the structure of Formula (I) is an ionizable lipidoid.

### Biodegradable Lipid Nanoparticles (LNPs)

In another aspect, the present disclosure provides a biodegradable lipid nanoparticle (LNP).

In certain embodiments, the LNP comprises (a) at least one compound of Formula (I).

In certain embodiments, the LNP comprises (b) at least one neutral phospholipid. In certain embodiments, the neutral phospholipid is present in a concentration range of about 5 mol % to about 45 mol %.

In certain embodiments, the LNP comprises (c) at least one cholesterol lipid. In certain embodiments, the total cholesterol lipid is in a concentration range of about 5 mol % to about 55 mol %.

In certain embodiments, the LNP comprises (d) at least one polyethylene glycol (PEG) or PEG-conjugated lipid. In certain embodiments, the PEG or PEG-conjugated lipid is in a concentration range of about 0.5 mol % to about 12.5 mol %.

In certain embodiments, the compound of Formula (I) comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP. In certain embodiments, the compound of Formula (I) comprises less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP. In certain embodiments, the compound of Formula (I) comprises more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the neutral phospholipid comprises about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP. In certain embodiments, the neutral phospholipid comprises less than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP. In certain embodiments, the neutral phospholipid comprises more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP. In certain embodiments, the neutral phospholipid comprises about 5, 6, 7, 8, 9, 10, 11, 12, 13, 14, 15, 16, 17, 18, 19, or about 20 mol % of the LNP. In certain embodiments, the neutral phospholipid comprises about 16 mol % of the LNP.

In certain embodiments, the at least one neutral phospholipid comprises dioleoyl-phosphatidylethanolamine (DOPE). In certain embodiments, the at least one neutral phospholipid comprises dioleoylphosphatidylcholine (DOPC). In certain embodiments, the at least one neutral phospholipid comprises distearoylphosphatidylcholine (DSPC). In certain embodiments, the at least one neutral phospholipid comprises distearoyl-phosphatidylethanolamine (DSPE). In certain embodiments, the at least one neutral phospholipid comprises 16-O-dimethyl PE. In certain embodiments, the at least one neutral phospholipid comprises 18-1-trans PE. In certain embodiments, the at least one neutral phospholipid comprises 1-stearioyl-2-oleoyl-phosphatidyethanol amine (SOPE). In certain embodiments, the at least one neutral phospholipid comprises stearoyloleoylphosphatidylcholine (SOPC). In certain embodiments, the at least one neutral phospholipid comprises N-(2,3-dioleoyloxy)propyl)-N,N,N-trimethylammonium chloride (DOTAP).

In certain embodiments, the at least one cholesterol lipid comprises about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or about 55 mol % of the LNP. In certain embodiments, the at least one cholesterol lipid comprises less than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or about 55 mol % of the LNP. In certain embodiments, the at least one cholesterol lipid comprises more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or about 55 mol % of the LNP. In certain embodiments, the at least one cholesterol lipid comprises about 40, 41, 42, 43, 44, 45, 46, 47, 48, 49, 50, 51, 52, 53, 54, or about 55 mol % of the LNP. In certain embodiments, the at least one cholesterol lipid comprises about 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, or about 55.0 mol % of the LNP. In certain embodiments, the at least one cholesterol lipid comprises about 46.5 mol % of the LNP.

In certain embodiments, the at least one cholesterol lipid comprises cholesterol. In certain embodiments, the at least one cholesterol lipid comprises cholesterol derivate.

In certain embodiments, the at least one PEG or PEG-conjugated lipid comprises 1,2-dimyristoyl-sn-glycero-3-phosphoethanolamine-N-[methoxy(polyethylene glycol)-2000](C14-PEG2000). In certain embodiments, the at least one PEG or PEG-conjugated lipid comprises C12-PEG2000. In certain embodiments, the at least one PEG or PEG-conjugated lipid comprises C12-PEG490. In certain embodiments, the at least one PEG or PEG-conjugated lipid comprises 1,2-dimyristoyl-rac-glycero-3-methoxypolyethylene glycol-2000 (DMG-PEG2000). In certain embodiments, the at least one PEG or PEG-conjugated lipid comprises 1,2-distearoyl-sn-glycero-3-phosphoethanolamine-N-[amino(polyethylene glycol)-2000](DSPE-PEG 2000 amine).

In certain embodiments, the molar ratio of (a):(b):(c):(d) is about 1-80:5-45:5-55:0.5-12.5. In certain embodiments, the molar ratio of (a):(b):(c):(d) is about 35-45:5-20:40-55:1-2.5. In certain embodiments, the molar ratio of (a):(b):(c):(d) is 30:16:46.5:2.5. In certain embodiments, the molar ratio of (a):(b):(c):(d) is 31:16:46.5:2.5, 32:16:46.5:2.5. In certain embodiments, the molar ratio of (a):(b):(c):(d) is 33:16:46.5:2.5. In certain embodiments, the molar ratio of (a):(b):(c):(d) is 34:16:46.5:2.5. In certain embodiments, the molar ratio of (a):(b):(c):(d) is 35:16:46.5:2.5.

In various embodiments, the LNP further comprises at least one helper compound. In various embodiments, the LNP comprises one or more helper compound in a concentration range of about 0 mol % to about 99.99 mol %. In some embodiments, the LNP comprises one or more helper compound in a concentration range of about 0.01 mol % to about 99.99 mol %. In some embodiments, the LNP comprises one or more helper compound in a concentration range of about 0.1 mol % to about 99.9 mol %. In some embodiments, the LNP comprises one or more helper compound in a concentration range of about 0.1 mol % to about 90 mol %. In some embodiments, the LNP comprises one or more helper compound in a concentration range of about 0.1 mol % to about 70 mol %. In some embodiments, the LNP comprises one or more helper compound in a concentration range of about 5 mol % to about 95 mol %. In some embodiments, the LNP comprises one or more helper compound in a concentration range of about 5 mol % to about 55 mol %. In some embodiments, the LNP comprises one or more helper compound in a concentration range of about 5 mol % to about 45 mol %. In some embodiments, the LNP comprises one or more helper compound in a concentration range of about 0.5 mol % to about 50 mol %. In some embodiments, the LNP comprises one or more helper compound in a concentration range of about 0.5 mol % to about 47 mol %. In some embodiments, the LNP comprises one or more helper compound in a concentration range of about 0.5 mol % to about 12.5 mol %. In some embodiments, the LNP comprises one or more helper compound in a concentration range of about 2.5 mol % to about 47 mol %.

For example, in some embodiments, the LNP comprises one or more helper compound in a concentration of about 0.01 mol %. In some embodiments, the LNP comprises one or more helper compound in a concentration of about 0.1 mol %. In some embodiments, the LNP comprises one or more helper compound in a concentration of about 0.5 mol %. In some embodiments, the LNP comprises one or more helper compound in a concentration of about 1 mol %. In some embodiments, the LNP comprises one or more helper compound in a concentration of about 1.5 mol %. In some embodiments, the LNP comprises one or more helper compound in a concentration of about 2 mol %. In some embodiments, the LNP comprises one or more helper compound in a concentration of about 2.5 mol %. In some embodiments, the LNP comprises one or more helper compound in a concentration of about 5 mol %. In some embodiments, the LNP comprises one or more helper compound in a concentration of about 10 mol %. In some embodiments, the LNP comprises one or more helper compound in a concentration of about 12 mol %. In some embodiments, the LNP comprises one or more helper compound in a concentration of about 15 mol %. In some embodiments, the LNP comprises one or more helper compound in a concentration of about 16 mol %. In some embodiments, the LNP comprises one or more helper compound in a concentration of about 20 mol %. In some embodiments, the LNP comprises one or more helper compound in a concentration of about 25 mol %. In some embodiments, the LNP comprises one or more helper compound in a concentration of about 30 mol %. In some embodiments, the LNP comprises one or more helper compound in a concentration of about 35 mol %. In some embodiments, the LNP comprises one or more helper compound in a concentration of about 37 mol %. In some embodiments, the LNP comprises one or more helper compound in a concentration of about 40 mol %. In some embodiments, the LNP comprises one or more helper compound in a concentration of about 45 mol %. In some embodiments, the LNP comprises one or more helper compound in a concentration of about 46.5 mol %. In some embodiments, the LNP comprises one or more helper compound in a concentration of about 47 mol %. In some embodiments, the LNP comprises one or more helper compound in a concentration of about 50 mol %. In some embodiments, the LNP comprises one or more helper compound in a concentration of about 60 mol %. In some embodiments, the LNP comprises one or more helper compound in a concentration of about 63 mol %. In some embodiments, the LNP comprises one or more helper compound in a concentration of about 70 mol %. In some embodiments, the LNP comprises one or more helper compound in a concentration of about 80 mol %. In some embodiments, the LNP comprises one or more helper compound in a concentration of about 90 mol %. In some embodiments, the LNP comprises one or more helper compound in a concentration of about 95 mol %. In some embodiments, the LNP comprises one or more helper compound in a concentration of about 95.5 mol %. In some embodiments, the LNP comprises one or more helper compound in a concentration of about 99 mol %. In some embodiments, the LNP comprises one or more helper compound in a concentration of about 99.99 mol %.

In some embodiments, the helper compound is a helper lipid, helper polymer, or any combination thereof. In some embodiments, the helper lipid is phospholipid (e.g. neutral phospholipid), cholesterol lipid, polymer, cationic lipid, neutral lipid, charged lipid, steroid, steroid analogue, polymer conjugated lipid, stabilizing lipid, or any combination thereof.

In some embodiments, the neutral phospholipid is dioleoyl-phosphatidylethanolamine (DOPE) or a derivative thereof, distearoylphosphatidylcholine (DSPC) or a derivative thereof, distearoyl-phosphatidylethanolamine (DSPE) or a derivative thereof, stearoyloleoylphosphatidylcholine (SOPC) or a derivative thereof, 1-stearioyl-2-oleoyl-phosphatidyethanol amine (SOPE) or a derivative thereof, N-(2,3-dioleoyloxy)propyl)-N,N,N-trimethylammonium chloride (DOTAP) or a derivative thereof, or any combination thereof. For example, in some embodiments, the LNP comprises a phospholipid in a concentration range of about 0 mol % to about 99.99 mol %. In some embodiments, the LNP comprises a neutral phospholipid in a concentration range of about 10 mol % to about 50 mol %. In some embodiments, the LNP comprises a phospholipid in a concentration range of about 5 mol % to about 45 mol %. In some embodiments, the LNP comprises a phospholipid in a concentration range of about 10 mol % to about 45 mol %. In some embodiments, the LNP comprises a phospholipid in a concentration range of about 16 mol % to about 40 mol %. In some embodiments, the LNP comprises a phospholipid in a concentration range of about 6 mol % to about 25 mol %. In some embodiments, the LNP comprises a phospholipid in a concentration range of about 5 mol % to about 20 mol %. In some embodiments, the LNP comprises a phospholipid in a concentration range of about 8 mol % to about 12 mol %. In some embodiments, the LNP comprises a phospholipid in a concentration range of about 6 mol % to about 12 mol %.

In some embodiments, the LNP comprises a phospholipid in a concentration of about 5 mol %, 6 mol %, 7 mol %, 8 mol %, 9 mol %, 10 mol %, 11 mol %, 12 mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, or 45 mol %. For example, in some embodiments, the LNP comprises a phospholipid in a concentration range of about 16 mol %. In some embodiments, the LNP comprises DOPE in a concentration of about 4 mol %. In some embodiments, the LNP comprises DOPE in a concentration of about 10 mol %. In some embodiments, the LNP comprises DOPE in a concentration of about 16 mol %. In some embodiments, the LNP comprises DOPE in a concentration of about 22 mol %. In some embodiments, the LNP comprises DOPE in a concentration of about 28 mol %.

In some embodiments, the cholesterol lipid is cholesterol and/or a derivative thereof, such as a substituted cholesterol molecule. In some embodiments, the LNP comprises a mixture of cholesterol and a substituted cholesterol molecule. For example, in some embodiments, the LNP comprises total cholesterol lipid including cholesterol and one or more substituted cholesterol in a concentration range of about 0 mol % to about 99.99 mol %. In some embodiments, the LNP comprises a total cholesterol lipid in a concentration range of about 1 mol % to about 99 mol %. In some embodiments, the LNP comprises a total cholesterol lipid in a concentration range of about 5 mol % to about 75 mol %. In some embodiments, the LNP comprises a total cholesterol lipid in a concentration range of about 5 mol % to about 55 mol %. In some embodiments, the LNP comprises a total cholesterol lipid in a concentration range of about 5 mol % to about 50 mol %. In some embodiments, the LNP comprises a total cholesterol lipid in a concentration range of about 20 mol % to about 50 mol %. In some embodiments, the LNP comprises total cholesterol lipid in a concentration range of about 20 mol % to about 47 mol %. In some embodiments, the LNP comprises a total cholesterol lipid in a concentration range of about 40 mol % to about 55 mol %.

In some embodiments, the LNP comprises a total cholesterol lipid in a concentration of about 5 mol %, 6 mol %, 7 mol %, 8 mol %, 9 mol %, 10 mol %, 11 mol %, 12 mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol %. For example, in some embodiments, the LNP comprises total cholesterol lipid in a concentration of about 29.5 mol %. In some embodiments, the LNP comprises total cholesterol lipid in a concentration of about 28.5 mol %. In some embodiments, the LNP comprises total cholesterol lipid in a concentration of about 35 mol %. In some embodiments, the LNP comprises total cholesterol lipid in a concentration of about 39.5 mol %. In some embodiments, the LNP comprises total cholesterol lipid in a concentration of about 46.5 mol %. In some embodiments, the LNP comprises total cholesterol lipid in a concentration of about 51 mol %. In some embodiments, the LNP comprises total cholesterol lipid in a concentration of about 51.5 mol %. In some embodiments, the LNP comprises total cholesterol lipid in a concentration of about 53.5 mol %.

In some embodiments, the polymer is one or more polyethylene glycol (PEG) lipids or PEG-conjugated lipids. Examples of such PEG or PEG-conjugated lipids include, but are not limited to, 1,2-dimyristoyl-sn-glycero-3-phosphoethanolamine-N-[methoxy(polyethylene glycol)-2000](C14-PEG2000) or a derivative thereof, 1,2-dimyristoyl-rac-glycero-3-methoxypolyethylene glycol-2000 (DMG-PEG2000) or a derivative, and/or 1,2-distearoyl-sn-glycero-3-phosphoethanolamine-N-[amino(poly ethylene glycol)-2000](DSPE-PEG 2000 amine) or a derivative. For example, in some embodiments, the LNP comprises a polymer in a concentration range of about 0 mol % to about 99.99 mol %. In some embodiments, the LNP comprises a polymer in a concentration range of about 0.1 mol % to about 25 mol %. In some embodiments, the LNP comprises a polymer in a concentration range of about 0.5 mol % to about 12.5 mol %. In some embodiments, the LNP comprises a polymer in a concentration range of about 0.5 mol % to about 3.5 mol %. In some embodiments, the LNP comprises a polymer in a concentration range of about 0.5 mol % to about 2.5 mol %. In some embodiments, the LNP comprises a polymer in a concentration range of about 1 mol % to about 2.5 mol %. For example, in some embodiments, the LNP comprises a polymer in a concentration about 0.5 mol %. In some embodiments, the LNP comprises a polymer in a concentration about 1.0 mol %. In some embodiments, the LNP comprises a polymer in a concentration about 1.5 mol %. In some embodiments, the LNP comprises a polymer in a concentration about 2.0 mol %. In some embodiments, the LNP comprises a polymer in a concentration about 2.5 mol %. In some embodiments, the LNP comprises a polymer in a concentration about 3.0 mol %. In some embodiments, the LNP comprises a polymer in a concentration about 3.5 mol %.

As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present disclosure. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In certain embodiments, the cationic lipid is an amino lipid. Suitable amino lipids useful in the disclosure include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In certain embodiments, the lipid is a PEGylated lipid, including, but not limited to, DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid.

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), distearoyl-phosphatidylethanolamine (DSPE)-maleimide-PEG, distearoyl-phosphatidylethanolamine (DSPE)-maleimide-PEG2000, 16-O-monomethyl PE, 16-O-dimethyl PE, 18-1-trans PE, 1-stearioyl-2-oleoyl-phosphatidyethanol amine (SOPE), stearoyloleoylphosphatidylcholine (SOPC), and 1,2-dielaidoyl-sn-glycero-3-phophoethanolamine (transDOPE). In certain embodiments, the neutral lipid is 1,2-distearoyl-sn-glycero-3-phosphocholine (DSPC).

In some embodiments, the composition comprises a neutral lipid selected from DSPC, DPPC, DSPE, SOPE, SOPC, DATAP, DMPC, DOPC, POPC, DOPE, and SM.

A “steroid” is a compound comprising the following carbon skeleton:

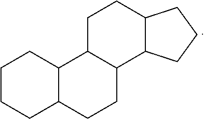 [SMILES: C1CCC2C(C1)CCC1C3CCCC3CCC21]

In certain embodiments, the steroid or steroid analogue is cholesterol. In some of these embodiments, the molar ratio of the cationic lipid.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include polyethylene glycol (PEG), maleimide PEG (mPEG), DSPE-PEG-DBCO, 1-(monomethoxy-polyethyleneglycol)-2,3-dimyristoylglycerol (PEG-s-DMG), DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

In certain embodiments, the LNP comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In certain embodiments, the polyethylene glycol-lipid is N-[(methoxy poly(ethylene glycol)₂₀₀₀)carbamyl]-1,2-dimyristyloxlpropyl-3-amine (PEG-c-DMA). In certain embodiments, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(w-methoxy(polyethoxy)ethyl)butanedioate (PEG-S-DMG), a pegylated ceramide (PEG-cer), or a PEG dialkoxypropylcarbamate such as w-methoxy(polyethoxy)ethyl-N-(2,3-di(tetradecanoxy)propyl)carbamate or 2,3-di(tetradecanoxy)propyl-N-(w-methoxy(polyethoxy)ethyl)carbamate.

In certain embodiments, the additional lipid is present in the LNP in an amount from about 1 mol % to about 10 mol %. In certain embodiments, the additional lipid is present in the LNP in an amount from about 1 mol % to about 5 mol %. In certain embodiments, the additional lipid is present in the LNP in about 1 mol % or about 2.5 mol %.

The term “lipid nanoparticle” refers to a particle having at least one dimension on the order of nanometers (e.g., 1-1,000 nm) which includes one or more lipids, for example a lipid of Formula (J)-(IV).

In various embodiments, the LNPs have a mean diameter of from about 30 nm to about 1500 nm, about 30 nm to about 1000 nm, about 30 nm to about 500 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 200 nm, 250 nm, 300 nm, 310 nm, 375 nm, 400 nm, 500 nm, 800 nm, 1000 nm, 1250 nm, 1400 nm, or 1500 nm. For example, in some embodiments, the LNPs have a mean diameter of from about 50 nm to about 200 nm.

In various embodiments, the lipids or the LNP of the present disclosure are substantially non-toxic.

In various embodiments, the lipids or the LNPs described herein are formulated for stability for in vivo cell targeting. For example, in some embodiments, the LNP formulated for stability for in vivo cell targeting comprises at least one compound having the structure of Formula (I) in a concentration range of about 0.1 mol % to about 99.99 mol %. In some embodiments, the at least one compound having the structure of Formula (I) is present in concentration range of about 1 mol % to about 45 mol %. In some embodiments, the at least one compound having the structure of Formula (I) is present in a concentration of about 40 mol %. In some embodiments, the at least one compound having the structure of Formula (I) is present in a concentration of about 30 mol %.

In some embodiments, the LNP formulated for stability for in vivo cell targeting comprises a phospholipid in a concentration range of about 10 mol % to about 45 mol %. In certain embodiments, the phospholipid is dioleoyl-phosphatidylethanolamine (DOPE), and the DOPE is present in a molar ratio of about 16 or at a molar percentage of about 16%.

In some embodiments, the LNP formulated for stability for in vivo a cell of interest (e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, and so forth) targeting comprises total cholesterol lipid in a concentration range of about 5 mol % to about 50 mol %. In certain embodiments, the total cholesterol is present in a molar ratio of about 46.5, or at a molar percentage of about 46.5%.

In some embodiments, the total cholesterol comprises a substituted cholesterol lipid. In some embodiments, the total cholesterol comprises a mixture of cholesterol and one or more substituted cholesterol lipid. In certain embodiments, the LNP molecule comprises total cholesterol at a ratio of 50% cholesterol:50% substituted cholesterol. In certain embodiments, the LNP molecule comprises total cholesterol at a ratio of 75% cholesterol:25% substituted cholesterol. In certain embodiments, the LNP molecule comprises total cholesterol at a ratio of 87.5% cholesterol:12.5% substituted cholesterol. In certain embodiments, the LNP molecule comprises total cholesterol at a ratio of 0% cholesterol:100% substituted cholesterol.

Exemplary substituted cholesterol lipids that can be incorporated into the LNP of the disclosure include, but are not limited to, a hydroxy substituted cholesterol, an epoxy substituted cholesterol and a keto substituted cholesterol.

In some embodiments, the substituted cholesterol lipid is 7α-hydroxycholesterol, 7β-hydroxycholesterol, 19-hydroxycholesterol, 20(S)-hydroxycholesterol, 24(S)-hydroxycholesterol, 25-hydroxycholesterol, 7-ketocholesterol, 5,6-epoxycholesterol, 3β, 5α, 6β-trihydroxycholesterol, 4β-hydroxycholesterol, 27-hydroxycholesterol or 22(R)-hydroxycholesterol.

By way of example, in certain embodiments, the LNP molecule comprises a mixture of 50% cholesterol:50% 7α-hydroxycholesterol. In certain embodiments, the LNP molecule comprises a mixture of 75% cholesterol:25% 7α-hydroxycholesterol.

In some embodiments, the LNP of the present disclosure comprises total PEG in a concentration range of about 0.5 mol % to about 12.5 mol %. In certain embodiments, the total PEG is present in a molar ratio of about 2.5, or at a molar percentage of about 2.5%.

In some embodiments, the PEG comprises a mixture of PEG maleimide PEG (mPEG).

In various embodiments, the LNP of the present disclosure comprises at least one compound having the structure of Formula (I), phospholipid, total cholesterol, and PEG-lipid, wherein the at least one compound having the structure of Formula (I): phospholipid:total cholesterol:PEG-lipid are present in a molar ratio of about 1-80:5-45:5-55:0.5-12.5 or at a molar percentage of about 1-80%:5-45%:5-55%:0.5-12.5%. In certain embodiments, the LNP comprises at least one compound having the structure of Formula (I), phospholipid. total cholesterol and PEG-lipid, wherein the at least one compound having the structure of Formula (I): phospholipid:total cholesterol:PEG-lipid are present in a molar ratio of about 35-45:5-20:40-55:1-2.5 or at a molar percentage of about 35-45%:5-20%:40-55%:1-2.5%. In certain embodiments, the LNP comprises at least one compound having the structure of Formula (I), phospholipid, total cholesterol and PEG-lipid, wherein the at least one compound having the structure of Formula (I): phospholipid:total cholesterol:PEG-lipid are present in a molar ratio of about 30-35:16:46.5:2.5 or at a molar percentage of about 35%:16%:46.5%:2.5%. In certain embodiments, the LNP comprises at least one compound having the structure of Formula (I), phospholipid, total cholesterol and PEG-lipid, wherein the at least one compound having the structure of Formula (I): phospholipid:total cholesterol: PEG-lipid are present in a molar ratio of about 35:16:46.5:2.5 or at a molar percentage of about 30-35%:16%:46.5%:2.5%.

In certain embodiments, the LNP comprises at least one compound having the structure of Formula (T), DOPE, total cholesterol, and PEG-conjugate, wherein the at least one compound having the structure of Formula (I):DOPE:total cholesterol:PEG-conjugate are present in a molar ratio of about 1-80:5-45:5-55:0.5-12.5 or at a molar percentage of about 1-80%:5-45%:5-55%:0.5-12.5%. In certain embodiments, the LNP comprises at least one compound having the structure of Formula (I), DOPE, total cholesterol and PEG, wherein the at least one compound having the structure of Formula (I):DOPE:total cholesterol:PEG are present in a molar ratio of about 35-45:5-20:40-55:1-2.5 or at a molar percentage of about 35-45%:5-20%:40-55%:1-2.5%. In certain embodiments, the LNP comprises at least one compound having the structure of Formula (I), DOPE, total cholesterol and PEG, wherein the at least one compound having the structure of Formula (I):DOPE:total cholesterol:PEG are present in a molar ratio of about 30-35:16:46.5:2.5 or at a molar percentage of about 35%:16%:46.5%:2.5%. In certain embodiments, the LNP comprises at least one compound having the structure of Formula (I), DOPE, total cholesterol and PEG, wherein the at least one compound having the structure of Formula (I):DOPE:total cholesterol:PEG are present in a molar ratio of about 35:16:46.5:2.5 or at a molar percentage of about 30-35%:16%:46.5%:2.5%.

Other exemplary molar ratios of the LNP components are provided in Table 1.

TABLE 1
Exemplary LNP Formulations

| Ionizable Lipid (a) | Phospholipid (b) | Cholesterol (c) | PEG-Lipid (d) |
|---|---|---|---|
| 40 | 4 | 53.5 | 2.5 |
| 60 | 10 | 29.5 | 0.5 |
| 40 | 28 | 28.5 | 3.5 |
| 40 | 28 | 29.5 | 2.5 |
| 40 | 22 | 35.0 | 3.0 |
| 35 | 22 | 40.0 | 3.0 |
| 35 | 22 | 39.5 | 3.5 |
| 30 | 16 | 51.0 | 3.0 |
| 30 | 16 | 51.5 | 2.5 |
| 40 | 16 | 42.5 | 1.5 |
| 35 | 16 | 46.5 | 2.5 |

In various embodiments, the LNP targets at least one cell of interest. For example, in some embodiments, the LNP targets at least one immune cell, stem cell, bone cell, blood cell, fat cell, endothelial cell, cancer cell, tissue cell, nerve cell, epithelial cell, connective tissue cell, muscle cell, or any combination thereof. In some embodiments, the LNP targets at least one brain cell, neuron, neuroglial cell, heart cell, liver cell, spleen cell, lung cell, kidney cell, podocytes, skin cell, keratinocyte, melanocyte, merkel cell, langerhans cell, cartilage cell, chondrocyte, pancreatic cell, skeletal muscle cell, cardiac muscle cell, smooth muscle cell, bone cell, osteoblast, osteoclast, osteocyte, lining cell, bone marrow cell, lymph node cell, white blood cell, granulocyte, neutrophil, eosinophil, basophil, agranulocyte, monocyte, lymphocyte, red blood cell, erythrocyte, platelet, fragments of megakaryocyte, embryonic stem cell, adult stem cell, mesenchymal stem cell, hematopoietic stem cell, white adipocyte, brown adipocyte, or any combination thereof.

In one aspect, the LNP comprises at least one cargo. In various aspects, the disclosure is not limited to any particular cargo or otherwise agent for which the biodegradable LNP is able to carry or transport. Rather, the disclosure includes any agent that can be carried by the biodegradable LNP. For example, agents that can be carried by the biodegradable LNP of the disclosure include, but are not limited to, diagnostic agents, detectable agents, and therapeutic agents. Thus, in various embodiments, the LNP comprises at least one agent. In other embodiments, the LNP encapsulates at least one agent.

In some embodiments, the LNP comprises, or encapsulates, at least one agent. In some embodiments, the weight ratio of (a):the at least one agent is between about 1:1 to about 10:1. In some embodiments, the LNP comprises, or encapsulates, at least one agent. In some embodiments, the weight ratio of (a):the at least one agent is between about 2:1 to about 10:1. In some embodiments, the LNP comprises, or encapsulates, at least one agent. In some embodiments, the weight ratio of (a):the at least one agent is between about 3:1 to about 10:1. In some embodiments, the LNP comprises, or encapsulates, at least one agent. In some embodiments, the weight ratio of (a):the at least one agent is between about 4:1 to about 10:1. In some embodiments, the LNP comprises, or encapsulates, at least one agent. In some embodiments, the weight ratio of (a):the at least one agent is between about 5:1 to about 10:1. In some embodiments, the LNP comprises, or encapsulates, at least one agent. In some embodiments, the weight ratio of (a):the at least one agent is between about 6:1 to about 10:1. In some embodiments, the LNP comprises, or encapsulates, at least one agent. In some embodiments, the weight ratio of (a):the at least one agent is between about 7:1 to about 10:1. In some embodiments, the LNP comprises, or encapsulates, at least one agent. In some embodiments, the weight ratio of (a):the at least one agent is between about 8:1 to about 10:1. In some embodiments, the LNP comprises, or encapsulates, at least one agent. In some embodiments, the weight ratio of (a):the at least one agent is between about 9:1 to about 10:1. In some embodiments, the LNP comprises, or encapsulates, at least one agent. In some embodiments, the weight ratio of (a):the at least one agent is between about 9.5:1 to about 10:1.

Thus, in various embodiments, the LNP is suitable for delivering at least one cargo to a cell of interest (e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, and so forth).

In another aspect, the present disclosure relates to a composition comprising at least one biodegradable compound or biodegradable LNP of the present disclosure. In one aspect, the present disclosure relates to a composition comprising at least one biodegradable compound or biodegradable LNP of the present disclosure that selectively targets at least one cell of interest. For example, in some embodiments, the composition targets at least one e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, or any combination thereof.

In one aspect, the composition of the present disclosure comprises one or more LNP formulated for targeted delivery of an agent to a cell of interest (e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, and so forth). Examples of such agents include, but are not limited to, a therapeutic agent, diagnostic agent, detectable agent, small molecule, peptide, polypeptide, amino acid molecule, nucleic acid molecule, drug, pro-drug, label, or any combination thereof.

For example, in some embodiments, the composition of the present disclosure comprises at least one therapeutic agent. In certain embodiments, the therapeutic agent is a hydrophobic therapeutic agent. In certain embodiments, the therapeutic agent is a hydrophilic therapeutic agent. Examples of such therapeutic agents include, but are not limited to, one or more drugs, proteins, amino acids, peptides, antibodies, antibiotics, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals. lipids, saccharides, metals, amino acids (and precursors), nucleic acids and precursors, contrast agents, diagnostic agents, dopamine receptor agonists, erectile dysfunction agents, fertility agents, gastrointestinal agents, hormones, inmunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, or any combinations thereof.

In certain embodiments, the therapeutic agent is one or more non-therapeutic moieties. In some embodiments, the nanoparticle comprises one or more therapeutic moieties, one or more non-therapeutic moieties, or any combination thereof. In certain embodiments, the therapeutic moiety targets cancer. In some embodiments, the composition comprises folic acid, peptides, proteins, aptamers, antibodies, small RNA molecules, miRNA, shRNA, siRNA, poorly water-soluble therapeutic agents, anti-cancer agents, or any combinations thereof.

In certain embodiments, the therapeutic agent may be an anti-cancer agent. Any suitable anti-cancer agent may be used in the compositions and methods of the present disclosure. The selection of a suitable anti-cancer agent may depend upon, among other things, the type of cancer to be treated and the nanoparticle compositions of the present disclosure. In certain embodiments, the anti-cancer agent may be effective for treating one or more of pancreatic cancer, esophageal cancer, rectal cancer, colon cancer, prostate cancer, kidney cancer, liver cancer, breast cancer, ovarian cancer, and stomach cancer. Examples of anti-cancer agents include, but is not limited to, chemotherapeutic agents, antiproliferative agents, anti-tumor agents, checkpoint inhibitors, and anti-angiogenic agents. For example, in certain embodiments, the anti-cancer agent is gemcitabine, doxorubicin, 5-Fu, tyrosine kinase inhibitors, sorafenib, trametinib, rapamycin, fulvestrant, ezalutamide, or paclitaxel.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The inhibitors of the disclosure can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2α; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-tnamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In certain embodiments, the anti-cancer drug is 5-fluorouracil, taxol, or leucovorin.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

In some embodiments, the LNP further comprises one or more gene components, such as siRNA or therapeutic DNA fragments. In some embodiments, the gene component is encapsulated in the LNP. In some embodiments, the gene component is on the surface of the LNP, for example, attached to or within the coating material.

In some embodiments, the LNP further comprises a biocompatible metal. Examples of biocompatible metals include, but are not limited to, copper, copper sulfide, iron oxide, cobalt and noble metals, such as gold and/or silver. One of ordinary skill in the art will be able to select of a suitable type of LNP taking into consideration at least the type of imaging and/or therapy to be performed.

### Small Molecule(s)

In various embodiments, the agent is a small molecule. In various embodiments, the agent is a therapeutic agent. In various embodiments, the therapeutic agent is a small molecule. When the therapeutic agent is a small molecule, a small molecule may be obtained using standard methods known to the skilled artisan. Such methods include chemical organic synthesis or biological means. Biological means include purification from a biological source, recombinant synthesis, and in vitro translation systems, using methods well known in the art. In certain embodiments, a small molecule therapeutic agents comprises an organic molecule, inorganic molecule, biomolecule, synthetic molecule, and the like.

Combinatorial libraries of molecularly diverse chemical compounds potentially useful in treating a variety of diseases and conditions are well known in the art, as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development. In some embodiments of the disclosure, the therapeutic agent is synthesized and/or identified using combinatorial techniques.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores. In some embodiments of the disclosure, the therapeutic agent is synthesized via small library synthesis.

The small molecule and small molecule compounds described herein may be present as salts even if salts are not depicted, and it is understood that the disclosure embraces all salts and solvates of the therapeutic agents depicted here, as well as the non-salt and non-solvate form of the therapeutic agents, as is well understood by the skilled artisan. In some embodiments, the salts of the therapeutic agents of the disclosure are pharmaceutically acceptable salts.

Where tautomeric forms may be present for any of the therapeutic agents described herein, each and every tautomeric form is intended to be included in the present disclosure, even though only one or some of the tautomeric forms may be explicitly depicted. For example, when a 2-hydroxypyridyl moiety is depicted, the corresponding 2-pyridone tautomer is also intended.

The disclosure also includes any or all of the stereochemical forms, including any enantiomeric or diastereomeric forms of the therapeutic agents described. The recitation of the structure or name herein is intended to embrace all possible stereoisomers of therapeutic agents depicted. All forms of the therapeutic agents are also embraced by the disclosure, such as crystalline or non-crystalline forms of the therapeutic agent. Compositions comprising a therapeutic agents of the disclosure are also intended, such as a composition of substantially pure therapeutic agent, including a specific stereochemical form thereof, or a composition comprising mixtures of therapeutic agents of the disclosure in any ratio, including two or more stereochemical forms, such as in a racemic or non-racemic mixture.

The disclosure also includes any or all active analog or derivative, such as a prodrug, of any therapeutic agent described herein. In certain embodiments, the therapeutic agent is a prodrug. In certain embodiments, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatives or analogs of known therapeutic agents, as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative of any of a small molecule inhibitor in accordance with the present disclosure can be used to treat a disease or disorder.

In certain embodiments, the small molecule therapeutic agents described herein can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Molecule(s)

In other related aspects, the agent is a nucleic acid molecule. In various embodiments, the agent is an isolated nucleic acid. Thus, in certain embodiments, an isolated nucleic acid, including for example a DNA oligonucleotide and a RNA oligonucleotide can be incorporated in the composition of the disclosure. In other related aspects, the therapeutic agent is an isolated nucleic acid. In certain embodiments, the isolated nucleic acid molecule is one of a DNA molecule or an RNA molecule. In certain embodiments, the isolated nucleic acid molecule is a cDNA, mRNA, siRNA, shRNA or miRNA molecule. In certain embodiments, the isolated nucleic acid molecule encodes a therapeutic peptide such a thrombomodulin, endothelial protein C receptor (EPCR), anti-thrombotic proteins including plasminogen activators and their mutants, antioxidant proteins including catalase, superoxide dismutase (SOD) and iron-sequestering proteins. In some embodiments, the therapeutic agent is an siRNA, miRNA, shRNA, or an antisense molecule, which inhibits a targeted nucleic acid including those encoding proteins that are involved in aggravation of the pathological processes.

In certain embodiments, the nucleic acid comprises a promoter/regulatory sequence such that the nucleic acid is capable of directing expression of the nucleic acid. Thus, the disclosure encompasses expression vectors and methods for the introduction of exogenous nucleic acid into cells with concomitant expression of the exogenous nucleic acid in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, PA (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216.

In one aspect, the disclosure includes a vector comprising an siRNA or an antisense polynucleotide. Preferably, the siRNA or antisense polynucleotide is capable of inhibiting the expression of a target polypeptide. The incorporation of a desired polynucleotide into a vector and the choice of vectors are well-known in the art as described in, for example, Sambrook et al. (2012), and in Ausubel et al. (1997), and elsewhere herein.

In certain embodiments, the expression vectors described herein encode a short hairpin RNA (shRNA) therapeutic agents. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleave the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells sought to be transfected or infected using a delivery vehicle of the disclosure. In other embodiments, the selectable marker may be carried on a separate piece of DNA and also be contained within the delivery vehicle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in one aspect, the delivery vehicle may contain a vector, comprising the nucleotide sequence or the construct to be delivered. The choice of the vector will depend on the host cell in which it is to be subsequently introduced. In a particular embodiment, the vector of the disclosure is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present disclosure to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the disclosure or the gene construct of the disclosure can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In certain embodiments, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, 0-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrawal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queuosine, and wybutosine and the like, as well as acetyl- methyl-, thio- and other modified forms of adenine, cytidine, guanine, thymine, and uridine.

In certain embodiments of the disclosure, an antisense nucleic acid sequence, which is expressed by a plasmid vector is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the disclosure may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the disclosure include oligonucleotide derivatives known in the art which have improved biological activity compared to unmodified oligonucleotides (see U.S. Pat. No. 5,023,243).

In certain embodiments of the disclosure, a ribozyme is used as a therapeutic agent to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents (Applied Biosystems, Inc., Foster City, CA) or they may be genetically expressed from DNA encoding them.

In certain embodiments, the therapeutic agent may comprise one or more components of a CRISPR-Cas system, where a guide RNA (gRNA) targeted to a gene encoding a target molecule, and a CRISPR-associated (Cas) peptide form a complex to induce mutations within the targeted gene. In certain embodiments, the therapeutic agent comprises a gRNA or a nucleic acid molecule encoding a gRNA. In certain embodiments, the therapeutic agent comprises a Cas peptide or a nucleic acid molecule encoding a Cas peptide.

In certain embodiments, the agent comprises a miRNA or a mimic of a miRNA. In certain embodiments, the agent comprises a nucleic acid molecule that encodes a miRNA or mimic of a miRNA.

miRNAs are small non-coding RNA molecules that are capable of causing posttranscriptional silencing of specific genes in cells by the inhibition of translation or through degradation of the targeted mRNA. A miRNA can be completely complementary or can have a region of non-complementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre- miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

In various embodiments, the agent comprises an oligonucleotide that comprises the nucleotide sequence of a disease-associated miRNA. In certain embodiments, the oligonucleotide comprises the nucleotide sequence of a disease-associated miRNA in a pre-microRNA, mature or hairpin form. In other embodiments, a combination of oligonucleotides comprising a sequence of one or more disease-associated miRNAs, any pre-miRNA, any fragment, or any combination thereof is envisioned.

MiRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. Desirable modifications are described, for example, in U.S. Patent Publication Nos. 20070213292, 20060287260, 20060035254, 20060008822. and 2005028824, each of which is hereby incorporated by reference in its entirety. For increased nuclease resistance and/or binding affinity to the target, the single- stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene- bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3′-terminus can be blocked with an aminoalkyl group. Other 3′ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose and so forth) can block 3′-5′-exonucleases.

In certain embodiments, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the ICSQ. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriesters, and boranophosphates. Various salts, mixed salts and free acid forms are also included.

A miRNA described herein, which may be in the mature or hairpin form, may be provided as a naked oligonucleotide. In some cases, it may be desirable to utilize a formulation that aids in the delivery of a miRNA or other nucleotide oligomer to cells (see, e.g., U.S. Pat. Nos. 5,656,611, 5,753,613, 5,785,992, 6,120,798, 6,221,959, 6,346,613, and 6,353,055, each of which is hereby incorporated by reference).

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration. A miRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg), salts, and RNAse inhibitors (e.g., a broad specificity RNAse inhibitor). In certain embodiments, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of a miRNA. In certain embodiments, the composition comprises oligonucleotides having nucleobase identity to the nucleobase sequence of a miRNA, and are thus designed to mimic the activity of the miRNA. In certain embodiments, the oligonucleotide composition that mimics miRNA activity comprises a double-stranded RNA molecule which mimics the mature miRNA hairpins or processed miRNA duplexes.

In certain embodiments, the oligonucleotide shares identity with endogenous miRNA or miRNA precursor nucleobase sequences. An oligonucleotide selected for inclusion in a composition of the present disclosure may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present disclosure encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA.

In the sense used in this description, a nucleotide sequence is “substantially homologous” to any of the nucleotide sequences describe herein when its nucleotide sequence has a degree of identity with respect to the nucleotide sequence of at least 60%, advantageously of at least 70%, preferably of at least 85%, and more preferably of at least 95%. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art. The identity between two amino acid sequences is preferably determined by using the BLASTN algorithm [BLAST Manual, Altschul, S., et al., NCBI NLM NIH Bethesda, Md. 20894, Altschul, S., et al., J. Mol. Biol. 215: 403-410 (1990)].

In certain embodiments, the composition comprises a nucleic acid molecule encoding a miRNA, precursor, mimic, or fragment thereof. For example, the composition may comprise a viral vector, plasmid, cosmid, or other expression vector suitable for expressing the miRNA, precursor, mimic, or fragment thereof in a desired mammalian cell or tissue.

### Polypeptide(s)

In other related aspects, the agent is a polypeptide. In various embodiments, the agent is an isolated polypeptide. In other related aspects, the therapeutic agent includes an isolated polypeptide. For example, in certain embodiments, the polypeptide of the disclosure inhibits or activates a target directly by binding to the target thereby modulating the normal functional activity of the target. In certain embodiments, the polypeptide of the disclosure modulates the target by competing with endogenous proteins. In certain embodiments, the polypeptide of the disclosure modulates the activity of the target by acting as a transdominant negative mutant.

The variants of the polypeptide therapeutic agents may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present disclosure, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

In one aspect, the disclosure includes an ionizable LNP molecule comprising or encapsulating one or more agent (e.g., a nucleic acid molecule) for targeted in vivo delivery of the encapsulated agent to a cell of interest (e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, and so forth). In certain embodiments, the nucleic acid molecule is a mRNA molecule.

In some embodiments, the mRNA molecule comprises a nucleotide sequence that can alternatively comprise sequence variations with respect to the original nucleotide sequences, for example, substitutions, insertions and/or deletions of one or more nucleotides, with the condition that the resulting polynucleotide encodes a polypeptide according to the disclosure.

As used herein, an amino acid sequence is “substantially homologous” to any of the amino acid sequences described herein when its amino acid sequence has a degree of identity with respect to the amino acid sequence of at least 60%, advantageously of at least 70%, preferably of at least 85%, and more preferably of at least 95%. The identity between two amino acid sequences is preferably determined by using the BLASTN algorithm (BLAST Manual, Altschul, S., et al., NCBI NLM NIH Bethesda, Md. 20894, Altschul, S., et al., J. Mol. Biol. 215: 403-410 (1990)).

In certain embodiments, the composition comprises a plurality of constructs, each construct encoding one or more antigens. In certain embodiments, the composition comprises 1 or more, 2 or more, 5 or more, 10 or more, 15 or more, or 20 or more constructs. In certain embodiments, the composition comprises a first construct, comprising a nucleotide sequence encoding an antigen; and a second construct, comprising a nucleotide sequence encoding an adjuvant.

In certain embodiments, the construct comprises a plurality of nucleotide sequences encoding a plurality of antigens. In certain embodiments, the construct encodes 1 or more, 2 or more, 5 or more, 10 or more, 15 or more, or 20 or more antigens. In certain embodiments, the disclosure relates to a construct, comprising a nucleotide sequence encoding an adjuvant. For example, in certain embodiments, the construct comprises a first nucleotide sequence encoding an antigen and a second nucleotide sequence encoding an adjuvant.

In another particular embodiment, the construct is operatively bound to a translational control element. The construct can incorporate an operatively bound regulatory sequence for the expression of the nucleotide sequence of the disclosure, thus forming an expression cassette.

### Peptide(s)

In certain embodiments, the agent is a peptide. Thus, in one aspect, a peptide can be incorporated into the LNP. Thus, in certain embodiments, the agent is a peptide. The peptide of the present disclosure may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present disclosure may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present disclosure, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present disclosure includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the disclosure can be post-translationally modified. For example, post-translational modifications that fall within the scope of the present disclosure include signal peptide cleavage, glycosylation, acetylation, isoprenylation, proteolysis, myristoylation, protein folding and proteolytic processing, and so forth. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the disclosure may include unnatural amino acids formed by post-translational modification or by introducing unnatural amino acids during translation.

### Antibodies

In certain embodiments, the agent is an antibody. Thus, in various embodiments, the composition of the disclosure comprises an antibody, or antibody fragment. In certain embodiments, the antibody targeting domain specifically binds to a target of interest. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity.

### Chimeric Antigen Receptor (CAR) Agent(s)

In certain embodiments, the agent comprises a nucleic acid sequence encoding a chimeric antigen receptor (CAR). In certain embodiments, the agent comprises an mRNA molecule encoding a CAR. In certain embodiments, the agent comprises a modified nucleoside mRNA molecule encoding a CAR.

In certain embodiments, a CAR comprises an extracellular domain capable of binding an antigen, including a tumor or pathogen antigen.

Targets of antigen-specific targeting regions of CARs may be of any kind. In some embodiments, the antigen-specific targeting region of the CAR targets antigens specific for cancer, inflammatory disease, neuronal-disorders, diabetes, cardiovascular disease, infectious diseases or a combination thereof. Examples of antigens that may be targeted by the CARs include but are not limited to antigens expressed on B-cells, antigens expressed on carcinomas, sarcomas, lymphomas, leukemia, germ cell tumors, blastomas, antigens expressed on various immune cells, and antigens expressed on cells associated with various hematologic diseases, autoimmune diseases, and/or inflammatory diseases. The CARs of the disclosure may be capable of redirecting the effector function of the expressing-cells to the target antigen(s).

Antigens that may be targeted by the CARs of the disclosure include but are not limited to any one or more of 4-IBB, 707-AP, 5T4, adenocarcinoma antigen, alpha-fetoprotein, BAFF, B-lymphoma cell, C242 antigen, CA-125, carbonic anhydrase 9 (CA-IX), C-MET, ART-4, BAGE, b-catenin/m, bcr-abl, CAMEL, CAP-1, CCR4, CD 152, CD7, CD 19, CD20, CD200, CD22, CD221, CD23 (IgE receptor), CD28. CD30 (TNFRSF8), CD33, CD4, CD38, CD40, CD44 v6, CD44v7/8, CD51, CD52, CD56, CD74, CD80, CD93, CD123, CD171, CEA, CLPP, CNT0888, CTLA-4, carcinoembryonic antigen, EGP2, EGP40, DR5, ErbB2, ErbB3/4, EGFR, EpCAM, EPV-E6, CD3, CASP-8, CD109, CDK/4, CDC-27, Cyp-B, DAM-8, DAM-10, ELV-M2, ETV6, FAP, fibronectin extra domain-B, folate receptor 1, GAGE, GD2, GD3 ganglioside, glycoprotein 75, GPNMB, G250, Gp100, HAGE, HER2/neu, HGF, HMW-MAA, human scatter factor receptor kinase, hTERT, IGF-1 receptor, IGF-I, IgG1, —I-CAM, IL-13, IL-6, insulin-like growth factor I receptor, integrin α5β1, integrin avP3, Kappa or light chain, LAGE, Lewis Y, G250/CAIX, Glypican-3, MAGE, MCl-R, mesothelin, MORAb-009, MS4A1, MUC1, MUC16, mucin CanAg, N-glycolylneuraminic acid, NPC-1C, PDGF-R a, PDL192, phosphatidylserine, PSC1, PSMA, NKG2D ligands, RANKL, RON, ROR1, SAGE, SCH 900105, SDC1, SLAMF7, TAG-72, TEL/AML, tenascin C, TGF beta 2, TGF-β, TRAIL-R1, TRAIL-R2, tumor antigen CTAA16.88, VEGF-A, VEGFR-1, VEGFR2, vimentin, B7-H6, IL-13 receptor a2, IL-11 receptor Ra, 8H9, NCAM, Fetal AchR, iCE, MART-1, tyrosinase, WT-1, TEM-1, TEM-2, TEM-3, TEM-4, TEM-5, TEM-6, TEM-7, TEM-8, ROBO-4, and so forth. Other antigens specific for cancer will be apparent to those of skill in the art and may be used in connection with alternate embodiments of the disclosure.

Particular examples of target antigens include but are not limited to surface proteins found on cancer cells in a specific or amplified fashion (e.g. the IL-14 receptor, CD 19, CD20 and CD40 for B-cell lymphoma, the Lewis Y and CEA antigens for a variety of carcinomas, the Tag72 antigen for breast and colorectal cancer, EGF-R for lung cancer, folate binding protein and the HER-2 protein that is often amplified in human breast and ovarian carcinomas), or viral proteins (e.g. gp120 and gp41 envelope proteins of HIV, envelope proteins from the Hepatitis B and C viruses, the glycoprotein B and other envelope glycoproteins of human cytomegalovirus, the envelope proteins from oncoviruses such as Kaposi's sarcoma-associated Herpes virus). Other targets of the CARs of the disclosure include CD4, where the ligand is the HIV gp120 envelope glycoprotein, and other viral receptors, for example ICAM, which is the receptor for the human rhinovirus, and the related receptor molecule for poliovirus.

In some embodiments, the bispecific chimeric antigen receptors target and bind at least two different antigens. Examples of pairings of at least two antigens bound by the bispecific CARs of the disclosure include but are not limited to any combination with HER2, CD 19 and CD20, CD 19 and CD22, CD20 and —I-CAM, —I-CAM and GD2, EGFR and —I-CAM, EGFR and C-MET, EGFR and HER2, C-MET and HER2 and EGFR and ROR1. Other pairings of antigens specific for cancer will be apparent to those of skill in the art and may be used in connection with alternate embodiments of the disclosure. In yet other embodiments, the bispecific chimeric antigen receptor targets CD 19 and CD20.

Antigens specific for inflammatory diseases that may be targeted by the CARs of the disclosure include but are not limited to any one or more of AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin a4, integrin α4β7, Lama glama, LFA-1 (CD11a), MEDI-528, myostatin, OX-40, rhuMAb (37, scleroscin, SOST, TGF beta 1, TNF-α or VEGF-A. Other antigens specific for inflammatory diseases will be apparent to those of skill in the art and may be used in connection with alternate embodiments of the disclosure.

Antigens specific for neuronal disorders that may be targeted by the CARs of the disclosure include but are not limited to any one or more of beta amyloid or MABT5102A. Other antigens specific for neuronal disorders will be apparent to those of skill in the art and may be used in connection with alternate embodiments of the disclosure.

Antigens specific for diabetes that may be targeted by the CARs of the disclosure include but are not limited to any one or more of L-43 or CD3. Other antigens specific for diabetes or other metabolic disorders will be apparent to those of skill in the art and may be used in connection with alternate embodiments of the disclosure.

Antigens specific for cardiovascular diseases which may be targeted by the CARs of the disclosure include but are not limited to any one or more of C5, cardiac myosin, CD41 (integrin alpha-lib), fibrin II, beta chain, ITGB2 (CD 18) and sphingosine-1-phosphate. Other antigens specific for cardiovascular diseases will be apparent to those of skill in the art and may be used in connection with alternate embodiments of the disclosure.

Antigens specific for infectious diseases that may be targeted by the CARs of the disclosure include but are not limited to any one or more of anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus and TNF-α. Other antigens specific for infectious diseases will be apparent to those of skill in the art and may be used in connection with alternate embodiments of the disclosure.

Additional targets of the CARs of the disclosure include antigens involved in B-cell associated diseases. Yet further targets of the CARs of the disclosure will be apparent to those of skill in the art and may be used in connection with alternate embodiments of the disclosure.

Other antigens specific for cancer will be apparent to those of skill in the art and may be used in connection with alternate embodiments of the disclosure.

In certain embodiments, the CAR comprises an antigen binding domain. In a particular non-limiting embodiment, the antigen-binding domain is an scFv specific for binding to a surface antigen of a target cell of interest (e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, and so forth).

In various embodiments, the CAR can be a “first generation,” “second generation,” “third generation,” “fourth generation” or “fifth generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

“First generation” CARs for use in the disclosure comprise an antigen binding domain, for example, a single-chain variable fragment (scFv), fused to a transmembrane domain, which is fused to a cytoplasmic/intracellular domain of the T cell receptor chain.

“First generation” CARs typically have the intracellular domain from the CD3ζ-chain, which is the primary transmitter of signals from endogenous T cell receptors (TCRs). “First generation” CARs can provide de novo antigen recognition and cause activation of both CD4+ and CD8+ T cells through their CD3ζ chain signaling domain in a single fusion molecule, independent of HLA-mediated antigen presentation.

“Second-generation” CARs for use in the disclosure comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3ζ signaling domain. Preclinical studies have indicated that “Second Generation” CARs can improve the anti-tumor activity of cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1(9):1577-1583 (2012)).

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD3ζ signaling domain in addition to a constitutive or inducible chemokine component.

“Fifth generation” CARs provide co-stimulation, for example, by CD28 or 4-1BB domains, and activation, for example, by a CD3ζ signaling domain, a constitutive or inducible chemokine component, and an intracellular domain of a cytokine receptor, for example, IL-2Rβ.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above.

### Adjuvant(s)

In certain embodiments, the agent is an adjuvant. Thus, in various embodiments, the composition comprises an adjuvant. In certain embodiments, the composition comprises a nucleic acid molecule encoding an adjuvant. In certain embodiments, the adjuvant-encoding nucleic acid molecule is IVT RNA. In certain embodiments, the adjuvant-encoding nucleic acid molecule is nucleoside-modified mRNA.

Exemplary adjuvants include, but is not limited to, alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. Other genes which may be useful adjuvants include those encoding: MCP-I, MIP-Ia, MIP-Ip, IL-8, RANTES, L-selectin, P-selectin, E-selectin, CD34, GlyCAM-1, MadCAM-1, LFA-I, VLA-I, Mac-1, p150.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R2, TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig and functional fragments thereof.

Alternatively, one or more of the agents below is delivered for therapeutic purposes as a sole agent and is not intended to function as an adjuvant to a co-administered compound. For example, the coding sequence for a gene therapy (e.g., replacement) of a desired protein may an agent delivered via an LNP as provided herein. Additionally, the coding sequence for a gene editing enzyme may be delivered. In these and other instances, the LNP may be formulated to minimize any immune response to the agent.

### Nucleoside-Modified RNA

In certain embodiments, the agent is a nucleoside-modified RNA. Thus, in one aspect, the composition comprises a nucleoside-modified RNA. Thus, in certain embodiments, the agent is a nucleoside-modified RNA In certain embodiments, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present disclosure is further described in U.S. Pat. No. 8,278,036, which is incorporated by reference herein in its entirety.

In certain embodiments, nucleoside-modified mRNA does not activate any pathophysiologic pathways, translates very efficiently and almost immediately following delivery, and serve as templates for continuous protein production in vivo lasting for several days (Karikó et al., 2008, Mol Ther 16:1833-1840; Karikó et al., 2012, Mol Ther 20:948-953). The amount of mRNA required to exert a physiological effect is small and that makes it applicable for human therapy.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable (Karikó et al., 2008, Mol Ther 16:1833-1840; Anderson et al., 2010, Nucleic Acids Res 38:5884-5892; Anderson et al., 2011, Nucleic Acids Research 39:9329-9338; Karikó et al., 2011, Nucleic Acids Research 39:e142; Karikó et al., 2012, Mol Ther 20:948-953; Karikó et al., 2005, Immunity 23:165-175).

It has been demonstrated that the presence of modified nucleosides, including pseudouridines in RNA suppress their innate immunogenicity (Karikó et al., 2005, Immunity 23:165-175). Further, protein-encoding, in vitro-transcribed RNA containing pseudouridine can be translated more efficiently than RNA containing no or other modified nucleosides (Karikó et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892). A preparative HPLC purification procedure has been established that was critical to obtain pseudouridine-containing RNA that has superior translational potential and no innate immunogenicity (Karikó et al., 2011, Nucleic Acids Research 39:e142). Administering HPLC-purified, pseudourine-containing RNA coding for erythropoietin into mice and macaques resulted in a significant increase of serum EPO levels (Karikó et al., 2012, Mol Ther 20:948-953), thus confirming that pseudouridine-containing mRNA is suitable for in vivo protein therapy.

The present disclosure encompasses RNA, oligoribonucleotide, and polyribonucleotide molecules comprising pseudouridine or a modified nucleoside. In certain embodiments, the composition comprises an isolated nucleic acid encoding an antigen or antigen binding molecule, wherein the nucleic acid comprises a pseudouridine or a modified nucleoside. In certain embodiments, the composition comprises a vector, comprising an isolated nucleic acid encoding an antigen, an antigen binding molecule, an adjuvant, or combination thereof, wherein the nucleic acid comprises a pseudouridine or a modified nucleoside.

In certain embodiments, the nucleoside-modified RNA of the disclosure is IVT RNA. For example, in certain embodiments, the nucleoside-modified RNA is synthesized by T7 phage RNA polymerase. In yet other embodiments, the nucleoside-modified mRNA is synthesized by SP6 phage RNA polymerase. In yet other embodiments, the nucleoside-modified RNA is synthesized by T3 phage RNA polymerase.

In certain embodiments, the modified nucleoside is m¹acp³Ψ (1-methyl-3-(3-amino-3-carboxypropyl) pseudouridine. In yet other embodiments, the modified nucleoside is m¹Ψ (1-methylpseudouridine). In yet other embodiments, the modified nucleoside is Ψm (2′-O-methylpseudouridine. In yet other embodiments, the modified nucleoside is m⁵D (5-methyldihydrouridine). In yet other embodiments, the modified nucleoside is m³Ψ(3-methylpseudouridine). In yet other embodiments, the modified nucleoside is a pseudouridine moiety that is not further modified. In yet other embodiments, the modified nucleoside is a monophosphate, diphosphate, or triphosphate of any of the above pseudouridines. In yet other embodiments, the modified nucleoside is any other pseudouridine-like nucleoside known in the art.

In yet other embodiments, the modified nucleoside of the present disclosure is m⁵C (5-methylcytidine). In yet other embodiments, the modified nucleoside is m⁵U (5-methyluridine). In yet other embodiments, the modified nucleoside is m⁶A (N⁶-methyladenosine). In yet other embodiments, the modified nucleoside is s²U (2-thiouridine). In yet other embodiments, the modified nucleoside is Ψ (pseudouridine). In yet other embodiments, the modified nucleoside is Um (2′-O-methyluridine).

In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i6A (2-methylthio-N⁶isopentenyladenosine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²₂Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); nCm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶₂Am (N⁶N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In yet other embodiments, a nucleoside-modified RNA of the present disclosure comprises a combination of 2 or more of the above modifications. In yet other embodiments, the nucleoside-modified RNA comprises a combination of 3 or more of the above modifications. In yet other embodiments, the nucleoside-modified RNA comprises a combination of more than 3 of the above modifications.

In yet other embodiments, between 0.1% and 100% of the residues in the nucleoside-modified of the present disclosure are modified (e.g. either by the presence of pseudouridine or a modified nucleoside base). In yet other embodiments, 0.1% of the residues are modified. In yet other embodiments, the fraction of modified residues is 0.2%. In yet other embodiments, the fraction is 0.3%. In yet other embodiments, the fraction is 0.4%. In yet other embodiments, the fraction is 0.5%. In yet other embodiments, the fraction is 0.6%. In yet other embodiments, the fraction is 0.8%. In yet other embodiments, the fraction is 1%. In yet other embodiments, the fraction is 1.5%. In yet other embodiments, the fraction is 2%. In yet other embodiments, the fraction is 2.5%. In yet other embodiments, the fraction is 3%. In yet other embodiments, the fraction is 4%. In yet other embodiments, the fraction is 5%. In yet other embodiments, the fraction is 6%. In yet other embodiments, the fraction is 8%. In yet other embodiments, the fraction is 10%. In yet other embodiments, the fraction is 12%. In yet other embodiments, the fraction is 14%. In yet other embodiments, the fraction is 16%. In yet other embodiments, the fraction is 18%. In yet other embodiments, the fraction is 20%. In yet other embodiments, the fraction is 25%. In y et other embodiments, the fraction is 30%. In yet other embodiments, the fraction is 35%. In yet other embodiments, the fraction is 40%. In yet other embodiments, the fraction is 45%. In yet other embodiments, the fraction is 50%. In yet other embodiments, the fraction is 60%. In yet other embodiments, the fraction is 70%. In yet other embodiments, the fraction is 80%. In yet other embodiments, the fraction is 90%. In yet other embodiments, the fraction is 100%.

In yet other embodiments, the fraction is less than 5%. In yet other embodiments, the fraction is less than 3%. In yet other embodiments, the fraction is less than 1%. In yet other embodiments, the fraction is less than 2%. In yet other embodiments, the fraction is less than 4%. In yet other embodiments, the fraction is less than 6%. In yet other embodiments, the fraction is less than 8%. In yet other embodiments, the fraction is less than 10%. In yet other embodiments, the fraction is less than 12%. In yet other embodiments, the fraction is less than 15%. In yet other embodiments, the fraction is less than 20%. In yet other embodiments, the fraction is less than 30%. In yet other embodiments, the fraction is less than 40%. In yet other embodiments, the fraction is less than 50%. In yet other embodiments, the fraction is less than 60%. In yet other embodiments, the fraction is less than 70%.

In yet other embodiments, 0.1% of the residues of a given nucleoside (i.e., uridine, cytidine, guanosine, or adenosine) are modified. In yet other embodiments, the fraction of the given nucleotide that is modified is 0.2%. In yet other embodiments, the fraction is 0.3%. In yet other embodiments, the fraction is 0.4%. In yet other embodiments, the fraction is 0.5%. In yet other embodiments, the fraction is 0.6%. In yet other embodiments, the fraction is 0.8%. In yet other embodiments, the fraction is 1%. In yet other embodiments, the fraction is 1.5%. In yet other embodiments, the fraction is 2%. In yet other embodiments, the fraction is 2.5%. In yet other embodiments, the fraction is 3%. In yet other embodiments, the fraction is 4%. In yet other embodiments, the fraction is 5%. In yet other embodiments, the fraction is 6%. In yet other embodiments, the fraction is 8%. In yet other embodiments, the fraction is 10%. In yet other embodiments, the fraction is 12%. In yet other embodiments, the fraction is 14%. In yet other embodiments, the fraction is 16%. In yet other embodiments, the fraction is 18%. In yet other embodiments, the fraction is 20%. In yet other embodiments, the fraction is 25%. In yet other embodiments, the fraction is 30%. In yet other embodiments, the fraction is 35%. In yet other embodiments, the fraction is 40%. In yet other embodiments, the fraction is 45%. In yet other embodiments, the fraction is 50%. In yet other embodiments, the fraction is 60%. In yet other embodiments, the fraction is 70%. In yet other embodiments, the fraction is 80%. In yet other embodiments, the fraction is 90%. In yet other embodiments, the fraction is 100%.

In yet other embodiments, the fraction of the given nucleotide that is modified is less than 8%. In yet other embodiments, the fraction is less than 10%. In yet other embodiments, the fraction is less than 5%. In yet other embodiments, the fraction is less than 3%. In yet other embodiments, the fraction is less than 1%. In yet other embodiments, the fraction is less than 2%. In yet other embodiments, the fraction is less than 4%. In yet other embodiments, the fraction is less than 6%. In yet other embodiments, the fraction is less than 12%. In yet other embodiments, the fraction is less than 15%. In yet other embodiments, the fraction is less than 20%. In yet other embodiments, the fraction is less than 30%. In yet other embodiments, the fraction is less than 40%. In yet other embodiments, the fraction is less than 50%. In yet other embodiments, the fraction is less than 60%. In yet other embodiments, the fraction is less than 70%.

In yet other embodiments, a nucleoside-modified RNA of the present disclosure is translated in the cell more efficiently than an unmodified RNA molecule with the same sequence. In yet other embodiments, the nucleoside-modified RNA exhibits enhanced ability to be translated by a target cell. In yet other embodiments, translation is enhanced by a factor of 2-fold relative to its unmodified counterpart. In yet other embodiments, translation is enhanced by a 3-fold factor. In yet other embodiments, translation is enhanced by a 5-fold factor. In yet other embodiments, translation is enhanced by a 7-fold factor. In yet other embodiments, translation is enhanced by a 10-fold factor. In yet other embodiments, translation is enhanced by a 15-fold factor. In yet other embodiments, translation is enhanced by a 20-fold factor. In yet other embodiments, translation is enhanced by a 50-fold factor. In yet other embodiments, translation is enhanced by a 100-fold factor. In yet other embodiments, translation is enhanced by a 200-fold factor. In yet other embodiments, translation is enhanced by a 500-fold factor. In yet other embodiments, translation is enhanced by a 1000-fold factor. In yet other embodiments, translation is enhanced by a 2000-fold factor. In yet other embodiments, the factor is 10-1000-fold. In yet other embodiments, the factor is 10-100-fold. In yet other embodiments, the factor is 10-200-fold. In yet other embodiments, the factor is 10-300-fold. In yet other embodiments, the factor is 10-500-fold. In yet other embodiments, the factor is 20-1000-fold. In yet other embodiments, the factor is 30-1000-fold. In yet other embodiments, the factor is 50-1000-fold. In yet other embodiments, the factor is 100-1000-fold. In yet other embodiments, the factor is 200-1000-fold. In yet other embodiments, translation is enhanced by any other significant amount or range of amounts.

In yet other embodiments, the nucleoside-modified antigen-encoding RNA of the present disclosure induces significantly more adaptive immune response than an unmodified in vitro-synthesized RNA molecule with the same sequence. In yet other embodiments, the modified RNA molecule exhibits an adaptive immune response that is 2-fold greater than its unmodified counterpart. In yet other embodiments, the adaptive immune response is increased by a 3-fold factor. In yet other embodiments the adaptive immune response is increased by a 5-fold factor. In yet other embodiments, the adaptive immune response is increased by a 7-fold factor. In yet other embodiments, the adaptive immune response is increased by a 10-fold factor. In yet other embodiments, the adaptive immune response is increased by a 15-fold factor. In yet other embodiments the adaptive immune response is increased by a 20-fold factor. In yet other embodiments, the adaptive immune response is increased by a 50-fold factor. In yet other embodiments, the adaptive immune response is increased by a 100-fold factor. In yet other embodiments, the adaptive immune response is increased by a 200-fold factor. In yet other embodiments, the adaptive immune response is increased by a 500-fold factor. In yet other embodiments, the adaptive immune response is increased by a 1000-fold factor. In yet other embodiments, the adaptive immune response is increased by a 2000-fold factor. In yet other embodiments, the adaptive immune response is increased by another fold difference.

In yet other embodiments, “induces significantly more adaptive immune response” refers to a detectable increase in an adaptive immune response. In yet other embodiments, the term refers to a fold increase in the adaptive immune response (e.g., 1 of the fold increases enumerated above). In yet other embodiments, the term refers to an increase such that the nucleoside-modified RNA can be administered at a lower dose or frequency than an unmodified RNA molecule with the same species while still inducing an effective adaptive immune response. In yet other embodiments, the increase is such that the nucleoside-modified RNA can be administered using a single dose to induce an effective adaptive immune response.

In yet other embodiments, the nucleoside-modified RNA of the present disclosure exhibits significantly less innate immunogenicity than an unmodified in vitro-synthesized RNA molecule with the same sequence. In yet other embodiments, the modified RNA molecule exhibits an innate immune response that is 2-fold less than its unmodified counterpart. In yet other embodiments, innate immunogenicity is reduced by a 3-fold factor. In yet other embodiments, innate immunogenicity is reduced by a 5-fold factor. In yet other embodiments, innate immunogenicity is reduced by a 7-fold factor. In yet other embodiments, innate immunogenicity is reduced by a 10-fold factor. In yet other embodiments, innate immunogenicity is reduced by a 15-fold factor. In yet other embodiments, innate immunogenicity is reduced by a 20-fold factor. In yet other embodiments, innate immunogenicity is reduced by a 50-fold factor. In yet other embodiments, innate immunogenicity is reduced by a 100-fold factor. In yet other embodiments, innate immunogenicity is reduced by a 200-fold factor. In yet other embodiments, innate immunogenicity is reduced by a 500-fold factor. In yet other embodiments, innate immunogenicity is reduced by a 1000-fold factor. In yet other embodiments, innate immunogenicity is reduced by a 2000-fold factor. In yet other embodiments, innate immunogenicity is reduced by another fold difference.

In yet other embodiments, “exhibits significantly less innate immunogenicity” refers to a detectable decrease in innate immunogenicity. In yet other embodiments, the term refers to a fold decrease in innate immunogenicity (e.g., 1 of the fold decreases enumerated above). In yet other embodiments, the term refers to a decrease such that an effective amount of the nucleoside-modified RNA can be administered without triggering a detectable innate immune response. In yet other embodiments, the term refers to a decrease such that the nucleoside-modified RNA can be repeatedly administered without eliciting an innate immune response sufficient to detectably reduce production of the recombinant protein. In yet other embodiments, the decrease is such that the nucleoside-modified RNA can be repeatedly administered without eliciting an innate immune response sufficient to eliminate detectable production of the recombinant protein.

In various embodiments, the composition comprises an in vitro transcribed (IVT) RNA molecule. For example, in certain embodiments, the composition of the disclosure comprises an IVT RNA molecule, which encodes an agent. In certain embodiments, the IVT RNA molecule of the present composition is a nucleoside-modified mRNA molecule.

In certain embodiments, the composition comprises at least one RNA molecule encoding a combination of at least two agents. In certain embodiments, the composition comprises a combination of two or more RNA molecules encoding a combination of two or more agents.

In certain embodiments, the present disclosure provides a method for gene editing of a cell of interest of a subject (e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, and so forth). For example, the method can be used to provide one or more component of a gene editing system (e.g., a component of a CRISPR system) to a cell of interest of a subject. In some embodiments, the method comprises administering to the subject a composition comprising one or more ionizable LNP molecule formulated for targeted delivery comprising one or more nucleoside-modified RNA molecule for gene editing.

In certain embodiments, the method comprises administration of the composition to a subject. In certain embodiments, the method comprises administering a plurality of doses to the subject. In yet other embodiments, the method comprises administering a single dose of the composition, where the single dose is effective in delivery of the target therapeutic agent.

In certain embodiments, the LNPs provided herein include a coding sequence for an editing enzyme encapsulated therein. Editing enzymes include various types of nucleases that are used to cut nucleic acid molecules. Such enzymes include zinc finger nucleases, Transcription activator-like effector nucleases (TALENs), meganucleases, clustered regularly interspaced short palindromic repeats (CRISPR) associated protein (CAS, e.g., CAS9), OMEGA enzymes (IscB), and so forth.

In certain embodiments, the nuclease is naturally occurring. In other embodiments, the nuclease is non-naturally occurring, i.e., engineered in the DNA-binding domain and/or cleavage domain. For example, the DNA-binding domain of a naturally-occurring nuclease may be altered to bind to a selected target site (e.g., CAS9 nuclease, a meganuclease that has been engineered to bind to site different than the cognate binding site). In other embodiments, the nuclease comprises heterologous DNA-binding and cleavage domains (e.g., zinc finger nucleases; TAL-effector nucleases; meganuclease DNA-binding domains with heterologous cleavage domains).

Zinc-finger nucleases (ZFNs) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms and serve as a prominent tool in the field of genome editing. In certain embodiments, the coding sequence encodes a zinc finger.

Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). In yet other embodiments, the coding sequence encodes a transcription activator-like (TAL) effector nuclease (TALEN).

In certain embodiments, the coding sequence encodes a CRISPR-associated nuclease (Cas9). “Cas9” (CRISPR associated protein 9) refers to family of RNA-guided DNA endonucleases which is characterized by two signature nuclease domains, RuvC (cleaves non-coding strand) and HNH (coding strand). Suitable bacterial sources of Cas9 include Staphylococcus aureus (SaCas9), Streptoococcus pyogenes (SpCas9), and Neisseria meningitides (K M Estelt et al, Nat Meth, 10:1116-1121 (2013), incorporated herein by reference). The wild-type coding sequences may be utilized in the constructs described herein. Alternatively, the bacterial codons are optimized for expression in humans, e.g., using any of a variety of known human codon optimizing algorithms. Alternatively, these sequences may be produced synthetically, either in full or in part. Other endonucleases with similar properties may optionally be substituted. See, e.g., the public CRISPR database (db) accessible at crispr dot u-psud dot fr/crispr.

In certain embodiments, the coding sequence encodes a meganuclease. Meganucleases are endodeoxyribonucleases characterized by a large recognition site (double-stranded DNA sequences of 12 to 40 base pairs), for example, I-SceI. When combined with a nuclease, DNA can be cut at a specific location. The restriction enzymes can be introduced into cells, for use in gene editing or for genome editing in situ. In certain embodiments, the nuclease is a member of the LAGLIDADG (SEQ ID NO: 1) family of homing endonucleases. In certain embodiments, the nuclease is a member of the I-CreI family of homing endonucleases which recognizes and cuts a 22 base pair recognition sequence SEQ ID NO: 2 (CAAAACGTCGTGAGACAGTTTG). See, e.g., WO 2009/059195. Methods for rationally-designing mono-LAGLIDADG homing endonucleases were described which are capable of comprehensively redesigning I-CreI and other homing endonucleases to target widely-divergent DNA sites, including sites in mammalian, yeast, plant, bacterial, and viral genomes (WO 2007/047859). The term “homing endonuclease” is synonymous with the term “meganuclease.” See, WO 2018/195449, describing certain PCSK9 meganucleases, which is incorporated herein in its entirety.

In certain embodiments, the compositions described herein include coding sequences for editing enzymes, particularly nucleases, which are useful targeting a gene for the insertion of a transgene. In certain situations, for example, for applications that do not require precise targeting of an existing gene or locus (e.g., to introduce or modify an endogenous gene, allele, or regulatory element), a common strategy is to target transgene integration to one of a small number of genomic “safe harbor” sites (SHS) for expression, presumably without disrupting the expression of adjacent or more distant genes. These putative SHS play an increasingly important role in developing effective gene therapies; in the investigation of gene structure, function, and regulation; and in cell-based biotechnology.

Certain SHS are known in the art, or may be discovered. Known SHS include the AAVS1 site on chromosome 19q, CCR5 chemokine receptor gene, ROSA26, PCKS9, and TTR. See, e.g., Monnat et al, New Human Chromosomal Sites with “Safe Harbor” Potential for Targeted Transgene Insertion, Hum Gene Ther. 2019 Jul. 1; 30(7): 814-828, which is incorporated by reference. In certain embodiments, the editing enzyme targets a SHS.

In certain embodiments, the editing enzyme is a nuclease that is specific for Proprotein convertase subtilisin/kexin type 9 (PCSK9). In other embodiments, the editing enzyme is a nuclease that is specific for transthyretin (TTR). See, e.g., Conway et al, Non-viral Delivery of Zinc Finger Nuclease mRNA Enables Highly Efficient In Vivo Genome Editing of Multiple Therapeutic Gene Targets, Molecular Therapy, 27(4):866-877 (April 2019), which is incorporated herein by reference. In certain embodiments, the nuclease is a meganuclease such as that described, e.g., in International Patent Publication No. WO 2018/195449.

In some embodiments, the LNP further includes sequences which direct the nuclease to a target site in the target locus. As used herein, the term “target site” or “target sequence” refers to the specific nucleotide sequence that is recognized by the editing enzyme, or its guide sequence. The “target locus” or “target gene locus” is any site in the gene coding region where insertion of the heterologous transgene is desired. For example, in certain embodiments, the target PCSK9 locus is in Exon 7 of the PCSK9 coding sequence located on chromosome 1.

In certain embodiments, such as a meganuclease specific for PCSK9 or TTR, no further sequences are required to direct the nuclease to the target site. However, in the case, for example, of Cas9, an additional sequence, called a “single guide RNA” or “sgRNA” is provided, which is specific for the target sequence. As used herein, the sgRNA has at least a 20-base sequence (or about 24-28 bases, sometimes called the seed region) for specific DNA binding (i.e., homologous to the target DNA), in combination with the gRNA scaffold. Transcription of sgRNAs should start precisely at the 5′ end. When targeting the template DNA strand, the base-pairing region of the sgRNA has the same sequence identity as the transcribed sequence. When targeting the non-template DNA strand, the base-pairing region of the sgRNA is the reverse-complement of the transcribed sequence. Optionally, the LNP may contain more than one sgRNA. The sgRNA is 5′ to a protospacer-adjacent motif (PAM) which is specifically recognized by the Cas9 (or Cpf1) enzyme. Typically, the sgRNA is “immediately” 5′ to the PAM sequence, i.e., there are no spacer or intervening sequences. Suitable sgRNAs can be designed by the person of skill in the art.

The sgRNA includes at least 20 nucleotides and specifically binds to a target site in the target gene, said target site being 5′ to a protospacer-adjacent motif (PAM) that is specifically recognized by the Cas9. The seed region in some embodiments shares 100% complementarity with the target site in the target gene. In other embodiments, the seed region contains 1, 2, 3, 4, or 5 mismatches as compared to the target site.

In other embodiments, for example, wherein the nuclease is a Cas9, the gene editing vector further includes one or more nuclear localization signal (NLSs). In certain embodiments, the NLSs flank the coding sequence for the Cas9.

In certain embodiments, the cargo is a DNA molecule or an RNA molecule. In certain embodiments, the cargo is a cDNA or mRNA molecule. In various embodiments, the composition comprises an in vitro transcribed (IVT) RNA molecule. For example, in certain embodiments, the composition comprises an IVT RNA molecule, which encodes an editing enzyme. In certain embodiments, the IVT RNA molecule is a nucleoside-modified mRNA molecule. In certain embodiments, where the nuclease coding sequence is provided as messenger RNA (mRNA). An mRNA may include a 5′ untranslated region, a 3′ untranslated region, and/or a coding or translating sequence.

An mRNA may be a naturally or non-naturally occurring mRNA. An mRNA may include one or more modified nucleobases, nucleosides, or nucleotides. In some embodiments, the mRNA in the compositions comprise at least one modification which confers increased or enhanced stability to the nucleic acid, including, for example, improved resistance to nuclease digestion in vivo. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine. As used herein, the terms “modification” and “modified” as such terms relate to the nucleic acids provided herein, include at least one alteration which preferably enhances stability and renders the mRNA more stable (e.g., resistant to nuclease digestion) than the wild-type or naturally occurring version of the mRNA. As used herein, the terms “stable” and “stability” as such terms relate to the nucleic acids of the present disclosure, and particularly with respect to the mRNA, refer to increased or enhanced resistance to degradation by, for example nucleases (i.e., endonucleases or exonucleases) which are normally capable of degrading such mRNA. Increased stability can include, for example, less sensitivity to hydrolysis or other destruction by endogenous enzymes (e.g., endonucleases or exonucleases) or conditions within the target cell or tissue, thereby increasing or enhancing the residence of such mRNA in the target cell, tissue, subject and/or cytoplasm. Also contemplated by the terms “modification” and “modified” as such terms related to the mRNA of the present disclosure are alterations which improve or enhance translation of mRNA nucleic acids, including for example, the inclusion of sequences which function in the initiation of protein translation (e.g., the Kozak consensus sequence).

In some embodiments, the mRNA described herein have undergone a chemical or biological modification to render them more stable. Exemplary modifications to an mRNA include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring mRNA, for example, covalent modifications such as the introduction of modified nucleotides, (e.g., nucleotide analogs, or the inclusion of pendant groups which are not naturally found in such mRNA molecules).

In some embodiments, the number of C and/or U residues in an mRNA sequence is reduced. In yet other embodiments, the number of C and/or U residues is reduced by substitution of one codon encoding a particular amino acid for another codon encoding the same or a related amino acid. Contemplated modifications to the mRNA nucleic acids of the present disclosure also include the incorporation of pseudouridines pseudouridine (y) or 5-methylcytosine (m⁵C). Substitutions and modifications to the mRNA of the present disclosure may be performed by methods readily known to one or ordinary skill in the art.

In certain embodiments, the mRNA includes a 5′ cap structure, a chain terminating nucleotide, a stem loop, a polyA sequence, and/or a polyadenylation signal. A cap structure or cap species is a compound including two nucleoside moieties joined by a linker and may be selected from a naturally occurring cap, a non-naturally occurring cap or cap analog, or an anti-reverse cap analog. An mRNA may instead or additionally include a chain terminating nucleoside.

In certain embodiments, the mRNA includes a stem loop, such as a histone stem loop. A stem loop may include 1, 2, 3, 4, 5, 6, 7, 8, or more nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

In certain embodiments, the mRNA includes a polyA sequence. A polyA sequence may be comprised entirely or mostly of adenine nucleotides or analogs or derivatives thereof. In certain embodiments, the polyA sequence is a tail located adjacent to a 3′ untranslated region of an mRNA.

In certain embodiments, the present disclosure provides a method for gene editing of a liver cell of interest of a subject (e.g., a liver cell). For example, the method can be used to provide one or more component of a gene editing system (e.g., a component of a CRISPR system) to a cell of interest of a subject. In certain embodiments, a second nucleic acid molecule is provided that encodes a transgene of interest, or an expression cassette containing the transgene coding sequence. In some embodiments, the transgene is a therapeutic agent. In certain embodiments, the transgene relates to a liver metabolic disorder. In certain embodiments, the transgene is OTC, PKU, CTLN1, or LDLR.

In certain embodiments, the transgene encodes a protein that is aberrantly expressed in a liver metabolic disorder or other genetic disorder. In certain embodiments, the transgene encodes a protein other than PCSK9. Such proteins include, but are not limited to OTC, low density lipoprotein receptor (LDLr), Factor IX, and. Factor VIII.

Further illustrative genes which may be delivered via the compositions described herein include, without limitation, glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxyacid Oxidase 1 (GO/HAO1) and AGXT, branched chain alpha-ketoacid dehydrogenase, including BCKDH, BCKDH-E2, BAKDH-E1a, and BAKDH-E1b, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type C1); propionic academia (PA); low density lipoprotein receptor (LDLR) protein, associated with familial hypercholesterolemia (FH), LDLR variant, such as those described in WO 2015/164778; ApoE and ApoC proteins, associated with dementia; lipoprotein lipase (LPL) (Lipoprotein Lipase Deficiency), UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; carnosinase (CN1); hypoxanthine-guanine phosphoribosyltransferase (HGPRT); erythropoietin (EPO); Carbamyl Phosphate Synthetase (CPS1), N-Acetylglutamate Synthetase (NAGS); Argininosuccinate Lyase (ASL) (Argininosuccinic Aciduria); and Arginase (AG); argininosuccsinate synthase associated with adult onset type II citrullinemia (CTLN2) (WO 2018/144709, which is incorporated herein by reference); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

Examples of suitable transgenes for delivery include, e.g., those associated with familial hypercholesterolemia (e.g., VLDLr, LDLr, ApoE, see, e.g., WO 2020/132155, WO 2018/152485, WO 2017/100682, which are incorporated herein by reference), muscular dystrophy, cystic fibrosis, and rare or orphan diseases. Examples of such rare disease may include spinal muscular atrophy (SMA), Huntingdon's Disease, Rett Syndrome (e.g., methyl-CpG-binding protein 2 (MeCP2); UniProtKB—P51608), Amyotrophic Lateral Sclerosis (ALS), Duchenne Type Muscular dystrophy, Friedrichs Ataxia (e.g., frataxin), progranulin (PRGN) (associated with non-Alzheimer's cerebral degenerations, including. frontotemporal dementia (FTD), progressive non-fluent aphasia (PNFA) and semantic dementia), among others. Other useful gene products include, carbamoyl synthetase I, ornithine transcarbamylase (OTC), arginosuccinate synthetase, arginosuccinate lyase (ASL) for treatment of arginosuccinate lyase deficiency, arginase, fumarylacetate hydrolase, phenylalanine hydroxylase, alpha-1 antitrypsin, rhesus alpha- fetoprotein (AFP), rhesus chorionic gonadotrophin (CG), glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)). Examples of suitable transgene for delivery may include human frataxin delivered in an AAV vector as described, e.g., PCT/US20/66167, Dec. 18, 2020, U.S. Provisional Patent Application No. 62/950,834, filed Dec. 19, 2019, and U.S. Provisional Application No. 63/136,059 filed on Jan. 11, 2021 which are incorporated herein by reference. Another example of suitable transgene for delivery may include human acid-α-glucosidase (GAA) delivered in an AAV vector as described, e.g., PCT/US20/30493, Apr. 30, 2020, now published as WO2020/223362A1, PCT/US20/30484, Apr. 20, 2020, now published as WO 2020/223356 A1, U.S. Provisional Patent Application No. 62/840,911, filed Apr. 30, 2019, US Provisional Application No. 62,913,401, filed Oct. 10, 2019, U.S. Provisional Patent Application No. 63/024,941, filed May 14, 2020, and U.S. Provisional Patent Application No. 63/109,677, filed Nov. 4, 2020 which are incorporated herein by reference. Also, another example of suitable transgene for delivery may include human a-L-iduronidase (IDUA) delivered in an AAV vector as described, e.g., PCT/US2014/025509, Mar. 13, 2014, now published as WO 2014/151341, and U.S. Provisional Patent Application No. 61/788,724, filed Mar. 15, 2013 which are incorporated herein by reference.

In certain embodiments, the transgene may be operably linked to regulatory sequences that direct the expression thereof. In some embodiments, the transgene cassette includes a promoter, the transgene coding sequence, and a poly A sequence. In some embodiments, the promoter is a liver-specific promoter, such as the TBG promoter, TBG-St promoter, HLP promoter, or others known in the art. In other embodiments, a transgene is provided without a promoter, and is inserted in the genome downstream of a native promoter, e.g., the PCSK9 promoter.

In addition to a promoter, the transgene cassette may contain one or more appropriate “regulatory elements” or “regulatory sequences”, which comprise but are not limited to an enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of suitable enhancers include, e.g., the alpha fetoprotein enhancer, the TTR minimal promoter/enhancer, LSP (TH-binding globulin promoter/alpha1-microglobulin/bikunin enhancer), amongst others. These control sequences or the regulatory sequences are operably linked to the nuclease coding sequences or transgene coding sequence.

In addition to the transgene cassette, in certain embodiments, the LNP composition described herein also includes homology-directed recombination (HDR) arms 5′ and 3′ to the transgene cassette, to facilitate homology directed recombination of the transgene into the endogenous genome. The homology arms are directed to the target locus and can be of varying length. In some embodiments, the HDR arms are from about 100 bp to about 1000 bp in length. In other embodiments, the HDR arms are from about 130 bp to about 500 bp. In other embodiments, the HDR arms are from about 100 bp to about 300 bp. In certain embodiments, the HDR arm is 130 bp. In other embodiments, the HDR arms are about 130 bp to 140 bp. In yet other embodiments, the HDR arms are about 500 bp. In yet other embodiments, the HDR arms are absent. The HDR arms ideally share a high level of complementarity with the target locus, although it need not be 100% complementarity. In some embodiments, 1, 2, 3, 4, 5, 6, 7, 8, 9, 10, 11, 12, 13, 14, 15, 16, 17, 18, 19, 20, or more mismatches are permitted in each HDR arm.

The ratio of ionizable lipid to nucleic acid may be varied in the LNP in a range from about 1:1 to about 10:1 by weight. In certain embodiments, the lipid:nucleic acid ratio is about 5:1. In certain embodiments, the lipid:nucleic acid ratio is about 6:1. In certain embodiments, the lipid:nucleic acid ratio is about 7:1. In certain embodiments, the lipid:nucleic acid ratio is about 8:1. In certain embodiments, the lipid:nucleic acid ratio is about 9:1. In certain embodiments, the lipid:nucleic acid ratio is about 10:1.

In embodiments where the composition includes a coding sequence for a Cas9 (e.g., mRNA encoding Cas9), the mRNA to sgRNA ratio can be present in a range of from about 1:5 to about 5:1 by weight. In certain embodiments, the mRNA:sgRNA ratio is about 1:5. In certain embodiments, the mRNA:sgRNA ratio is about 1:4. In certain embodiments, the mRNA:sgRNA ratio is about 1:3. In certain embodiments, the mRNA:sgRNA ratio is about 1:2. In certain embodiments, the mRNA:sgRNA ratio is about 1:1. In certain embodiments, the mRNA:sgRNA ratio is about 2:1. In certain embodiments, the mRNA:sgRNA ratio is about 3:1. In certain embodiments, the mRNA:sgRNA ratio is about 4:1. In certain embodiments, the mRNA:sgRNA ratio is about 5:1. Other ratios within this range can be utilized.

LNP formation and encapsulation of cargo may be accomplished using techniques known in the art. See, e.g., Jeffs, et al (March 2005). A Scalable, Extrusion-Free Method for Efficient Liposomal Encapsulation of Plasmid DNA. Pharmaceutical Research, 22(3), 362-372, and Kulkari et al, On the Formation and Morphology of Lipid Nanoparticles Containing Ionizable Cationic Lipids and siRNA, ACS Nano, 12:4787-4795 (April 2018) both of which are incorporated herein by reference. For example, in brief, LNP-mRNA compositions are generated by rapid mixing of lipids in ethanol with mRNA in aqueous buffer (pH 4.0), followed by dialysis to remove ethanol and to raise the pH to 7.4.

### Combinations

In certain embodiments, the composition of the present disclosure comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, in certain embodiments, the composition comprises a 1:1 ratio of two individual agents. However, the combination is not limited to any particular ratio. Rather any ratio that is shown to be effective is encompassed.

### Methods

The present disclosure provides methods of delivering an agent to a cell of interest of a target subject. Exemplary cells that can be targeted using the LNP compositions of the disclosure include, but are not limited to, an immune cell, stem cell, bone cell, blood cell, fat cell, endothelial cell, cancer cell, tissue cell, nerve cell, epithelial cell, connective tissue cell, and/or muscle cell (e.g., a heart cell, liver cell, spleen cell, lung cell, kidney cell, podocytes, skin cell, cartilage cell, brain cell, white blood cell, hematopoietic stem cell, or any combination thereof).

In some embodiments, the agent is a diagnostic agent to detect at least one marker associated with a disease or disorder. In some embodiments, the agent is a therapeutic agent for the treatment or prevention of a disease or disorder. In some embodiments, the agent is an editing enzyme for gene editing. Therefore, in some embodiments, the disclosure provides methods for diagnosing, treating, ameliorating, and/or preventing a disease or disorder comprising administering an effective amount of the LNP composition comprising one or more diagnostic or therapeutic agents, one or more adjuvants, or a combination thereof.

For example, in certain embodiments, the disease or disorder is a liver disease or disorder, pulmonary disease or disorder, spleen disease or disorder, renal disease or disorder, heart disease or disorder, cardiovascular disease or disorder, brain disease or disorder, neurological disease or disorder, cancer, bone disease or disorder, bone marrow disease or disorder, skin disease or disorder, connective tissue disease or disorder, pancreatic disease or disorder, muscle disease or disorder, lymph node disease or disorder, blood disease or disorder, or any combination thereof.

In some embodiments, the disclosure relates to methods of treating, ameliorating, and/or preventing cardiovascular conditions and diseases or disorders associated therewith in subjects in need thereof, the method comprising administering the LNP composition of the disclosure. Exemplary cardiovascular conditions that can be treated using the LNP compositions and methods of the disclosure include, but are not limited to, hypertrophic cardiomyopathy, dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertensive heart disease, arrhythmogenic right ventricular cardiomyopathy (ARVC), tubulointerstitial and glomerular fibrosis, atherosclerosis, varicose veins, cerebral infarcts, or any combination thereof.

In some embodiments, the disclosure relates to methods of treating, ameliorating, and/or preventing liver diseases or disorders and diseases or disorders associated therewith in subjects in need thereof, the method comprising administering the LNP composition of the disclosure. Exemplary liver diseases or disorders that can be treated using the LNP compositions and methods of the disclosure include, but are not limited to, hepatitis A, hepatitis B, hepatitis C, autoimmune hepatitis, primary biliary cholangitis, primary sclerosing cholangitis, hemochromatosis, Wilson's disease, alpha-1 antitrypsin deficiency, liver cancer, bile duct cancer, liver adenoma, transthyretin (TTR), proprotein convertase subtilisin/kexin type 9 (PCSK9)-based diseases or disorders, or any combination thereof.

Further exemplary diseases or disorders that can be treated using the LNP compositions and methods of the disclosure include, but are not limited to, glycogen storage disease or deficiency type TA (GSD1), phosphoenolpyruvate carboxykinase (PEPCK) deficiency, cyclin-dependent kinase-like 5 (CDKL5) deficiency, galactosemia, phenylketonuria (PKU), primary hyperoxaluria type 1, maple syrup urine disease, tyrosinemia type 1, methylmalonic acidemia, medium chain acetyl CoA deficiency, ornithine transcarbamylase deficiency, citrullinemia, lecithin-cholesterol acyltransferase (LCAT) deficiency, amethylmalonic acidemia (MMA), Niemann-Pick disease, propionic academia (PA), familial hypercholesterolemia (FH), dementia, Lipoprotein Lipase Deficiency, Crigler-Najjar disease, severe combined immunodeficiency disease, Gout and Lesch-Nyan syndrome, biotimidase deficiency, Fabry disease, GM1 gangliosidosis, Gaucher disease type 2 and 3, Zellweger syndrome, metachromatic leukodystrophy, Krabbe disease, Pompe disease, Nieman Pick disease type A, Argininosuccinic Aciduria, adult onset type II citrullinemia, urea cycle disorders, Farber lipogranulomatosis, aspartyl-glucosaminuria, fucosidosis, alpha-mannosidosis, acute intermittent porphyria (AIP), alpha-1 antitrypsin deficiency (emphysema), anemia due to thalassemia or to renal failure, ischemic diseases, occluded blood vessels as seen in, for example, atherosclerosis, thrombosis, or embolisms, Parkinson's disease, congestive heart failure, muscular dystrophies, and diabetes.

In some embodiments, the disclosure relates to methods of treating, ameliorating, and/or preventing spleen diseases or disorders in subjects in need thereof, the method comprising administering the LNP composition of the disclosure. Exemplary spleen diseases or disorders that can be treated using the LNP compositions and methods of the disclosure include, but are not limited to, damaged or ruptured spleen, enlarged spleen, or any combination thereof.

In some embodiments, the disclosure relates to methods of treating, ameliorating, and/or preventing pulmonary diseases or disorders and diseases or disorders associated therewith in subjects in need thereof, the method comprising administering the LNP composition of the disclosure. Exemplary pulmonary diseases or disorders that can be treated using the LNP compositions and methods of the disclosure include, but are not limited to, asthma, chronic obstructive pulmonary disease (COPD), interstitial lung disease (ILD), pulmonary embolism(PE), pulmonary hypertension, pleural effusion, pneumothorax, mesothelioma, obesity hypoventilation syndrome, neuromuscular disorders, bronchitis, chronic bronchitis, acute bronchitis, emphysema, cystic fibrosis, pneumonia, pneumoconiosis, tuberculosis, pulmonary edema, lung cancer, acute respiratory distress syndrome (ARDS), pulmonary lymphangioleiomyomatosis, or any combination thereof.

In some embodiments, the disclosure relates to methods of treating, ameliorating, and/or preventing renal diseases or disorders in subjects in need thereof, the method comprising administering the LNP composition of the disclosure. Exemplary renal diseases or disorders that can be treated using the LNP compositions and methods of the disclosure include, but are not limited to, renal fibrosis, nephritic syndrome, Alport's syndrome, HIV associated nephropathy, polycystic kidney disease, Fabry's disease, diabetic nephropathy, chronic glomerulonephritis, nephritis associated with systemic lupus); progressive systemic sclerosis (PSS), chronic graft versus host disease, or any combination thereof.

In some embodiments, the disclosure relates to methods of treating, ameliorating, and/or preventing neurological diseases or disorders in subjects in need thereof, the method comprising administering the LNP composition of the disclosure. Exemplary neurological diseases or disorders that can be treated using the LNP compositions and methods of the disclosure include, but are not limited to, acute spinal cord injury, Alzheimer's disease, amyotrophic lateral sclerosis (ALS), ataxia, bell's palsy, brain tumor, cerebral aneurysm, epilepsy, seizure, Guillain-Barre syndrome, headache, migrane, head injury, hydrocephalus, lumbar disk disease (herniated disk), meningitis, multiple sclerosis, muscular dystrophy, neurocutaneous syndrome, Parkinson's disease, stroke, cluster headache, tension headache, migraine headaches, encephalitis, or any combination thereof.

In some embodiments, the disclosure relates to methods of treating, ameliorating, and/or preventing bone diseases or disorders in subjects in need thereof, the method comprising administering the LNP composition of the disclosure. Exemplary bone diseases or disorders that can be treated using the LNP compositions and methods of the disclosure include, but are not limited to, osteoporosis, fracture, scoliosis, Paget's disease, osteoarthritis, rheumatoid arthritis, gout, bursitis, solid tumor cancer that metastasizes to bone, or any combination thereof.

In some embodiments, the disclosure relates to methods of treating, ameliorating. and/or preventing bone marrow diseases or disorders in subjects in need thereof, the method comprising administering the LNP composition of the disclosure. Exemplary bone marrow diseases or disorders that can be treated using the LNP compositions and methods of the disclosure include, but are not limited to, leukemia, myelodysplastic syndrome (MDS), myeloproliferative disorders (MPD), aplastic anemia, iron deficiency anemia, disorders of plasma cells, plasma cell dyscrasia, lymphomas, thrombotic thrombocytopenic purpura, a disease or disorder arising from hematopoietic stem cells, or any combination thereof.

In some embodiments, the disclosure relates to methods of treating, ameliorating, and/or preventing skin diseases or disorders in subjects in need thereof, the method comprising administering the LNP composition of the disclosure. Exemplary skin diseases or disorders that can be treated using the LNP compositions and methods of the disclosure include, but are not limited to, eczema, cold sores, dry skin, psoriasis, vitiligo, contact dermatitis, rosacea, melasma, wart, actinic keratosis, melanoma, or any combination thereof.

In some embodiments, the disclosure relates to methods of treating, ameliorating, and/or preventing connective tissue diseases or disorders in subjects in need thereof, the method comprising administering the LNP composition of the disclosure. Exemplary connective tissue diseases or disorders that can be treated using the LNP compositions and methods of the disclosure include, but are not limited to, rheumatoid arthritis (RA), scleroderma, granulomatosis with polyangiitis (GPA), Churg-Strauss syndrome, systemic lupud erythematosus (SLE), miscoscopic polyangiitis (MPA), polymyositis, dermatomyositis, or any combination thereof.

In some embodiments, the disclosure relates to methods of treating, ameliorating, and/or preventing pancreatic diseases or disorders in subjects in need thereof, the method comprising administering the LNP composition of the disclosure. Exemplary pancreatic diseases or disorders that can be treated using the LNP compositions and methods of the disclosure include, but are not limited to, pancreatitis, acute pancreatitis, chronic pancreatitis, hereditary pancreatitis, pancreatic cancer, diabetes, or any combination thereof.

In some embodiments, the disclosure relates to methods of treating, ameliorating, and/or preventing muscle diseases or disorders in subjects in need thereof, the method comprising administering the LNP composition of the disclosure. Exemplary muscle diseases or disorders that can be treated using the LNP compositions and methods of the disclosure include, but are not limited to, muscular dystrophy, Duchenne muscular dystrophy (DMD), Becker muscular dystrophy (BMD), myasthenia gravis, movement disorder, muscle cramps, myositis, or any combination thereof.

In some embodiments, the disclosure relates to methods of treating, ameliorating, and/or preventing lymph node diseases or disorders in subjects in need thereof, the method comprising administering the LNP composition of the disclosure. Exemplary lymph node diseases or disorders that can be treated using the LNP compositions and methods of the disclosure include, but are not limited to, lymphatic system disease or disorder, lymphoma, castleman disease, lymphangiomatosis, lymphatic filariasis, tonsillolith, solid tumor cancer that metastasized to lymph nodes, or any combination thereof.

In some embodiments, the disclosure relates to methods of treating, ameliorating, and/or preventing blood diseases or disorders in subjects in need thereof, the method comprising administering the LNP composition of the disclosure. Exemplary blood diseases or disorders that can be treated using the LNP compositions and methods of the disclosure include, but are not limited to, anemia, hemophilia, leukocytosis, polycythemia vera, sickle cell disease, thalassemia, Von Willebrand disease, or any combination thereof.

In some embodiments, the disclosure relates to methods of treating, ameliorating, and/or preventing cancer and diseases or disorders associated therewith in subjects in need thereof, the method comprising administering the LNP composition of the disclosure.

In some embodiments, the present disclosure provides a method for inducing an immune response in subjects in need thereof, the method comprising administering the LNP composition of the disclosure. For example, in certain embodiments, the method for inducing an immune response in subjects in need thereof is a cancer immunotherapy comprising administering the LNP comprising CAR to the subject to induce an immune response against cancer.

Exemplary cancers that can be treated using the LNP compositions and methods of the disclosure include, but are not limited to, acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

In various embodiments, the disease or disorder is a disease or disorder associated with at least one cell of interest (e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, and so forth). For example, in certain embodiments, the disease or disorder associated with at least one cell of interest (e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, and so forth) is a liver disease or disorder, pulmonary disease or disorder, spleen disease or disorder, renal disease or disorder, heart disease or disorder, cardiovascular disease or disorder, brain disease or disorder, neurological disease or disorder, cancer, bone disease or disorder, bone marrow disease or disorder, skin disease or disorder, connective tissue disease or disorder, pancreatic disease or disorder, muscle disease or disorder, lymph node disease or disorder, blood disease or disorder, or any combination thereof.

In certain embodiments, the method comprises administering a LNP composition of the disclosure comprising one or more nucleic acid molecules for treatment, amelioration, and/or prevention of a disease or disorder (e.g., a liver disease or disorder, pulmonary disease or disorder, spleen disease or disorder, renal disease or disorder, heart disease or disorder, cardiovascular disease or disorder, brain disease or disorder, neurological disease or disorder, cancer, bone disease or disorder, bone marrow disease or disorder, skin disease or disorder, connective tissue disease or disorder, pancreatic disease or disorder, muscle disease or disorder, lymph node disease or disorder, blood disease or disorder, or any combination thereof). In certain embodiments, the one or more nucleic acid molecules encode a therapeutic agent for the treatment, amelioration, and/or prevention of the disease or disorder (e.g., a liver disease or disorder, pulmonary disease or disorder, spleen disease or disorder, renal disease or disorder, heart disease or disorder, cardiovascular disease or disorder, brain disease or disorder, neurological disease or disorder, cancer, bone disease or disorder, bone marrow disease or disorder, skin disease or disorder, connective tissue disease or disorder, pancreatic disease or disorder, muscle disease or disorder, lymph node disease or disorder, blood disease or disorder, or any combination thereof).

In certain embodiments, the compositions of the disclosure can be administered in combination with one or more additional therapeutic agent, an adjuvant, or a combination thereof. For example, in certain embodiments, the method comprises administering an LNP composition comprising a nucleic acid molecule encoding one or more agent for targeted administration to a cell of interest (e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, and so forth) and a second LNP comprising a nucleic acid molecule encoding one or more adjuvants. In certain embodiments, the method comprises administering a single LNP composition comprising a nucleic acid molecule encoding one or more agent for targeted administration to a cell of interest e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, and so forth) and a nucleic acid molecule encoding one or more adjuvants.

In certain embodiments, the method comprises administering to subject a plurality of LNPs of the disclosure comprising nucleoside-modified nucleic acid molecules encoding a plurality of agents to a cell of interest (e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, and so forth), adjuvants, or a combination thereof.

In certain embodiments, the method comprises administering the LNP of the disclosure comprising nucleoside-modified RNA, which provides stable expression of a nucleic acid encoded agent (e.g., a therapeutic agent encoded by a nucleoside modified mRNA molecule) described herein to a cell of interest e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, and so forth).

Administration of the compositions of the disclosure in a method of treatment, amelioration, and/or prevention can be achieved in a number of different ways, using methods known in the art. In certain embodiments, the method of the disclosure comprises systemic administration of the subject, including for example enteral or parenteral administration. In certain embodiments, the method comprises intradermal delivery of the composition. In yet other embodiments, the method comprises intravenous delivery of the composition. In some embodiments, the method comprises intramuscular delivery of the composition. In certain embodiments, the method comprises subcutaneous delivery of the composition. In certain embodiments, the method comprises inhalation of the composition. In certain embodiments, the method comprises intranasal delivery of the composition.

It will be appreciated that the composition of the disclosure may be administered to a subject either alone, or in conjunction with another agent.

The therapeutic and prophylactic methods of the disclosure thus encompass the use of pharmaceutical compositions comprising at least one LNP of the disclosure comprising an agent (e.g., an mRNA molecule) described herein, to practice the methods of the disclosure. The pharmaceutical compositions useful for practicing the disclosure may be administered to deliver a dose of from 0.001 ng/kg/day and 100 mg/kg/day. For example, in some embodiments, the pharmaceutical compositions useful for practicing the disclosure may be administered to deliver a dose of from 0.005 mg/kg/day and 5 mg/kg/day. In certain embodiments, the disclosure envisions administration of a dose which results in a concentration of the compound of the present disclosure from 10 nM and 10 μM in a mammal.

Typically, dosages which may be administered in a method of the disclosure to a mammal, preferably a human, range in amount from 0.01 g to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 0.1 g to about 10 mg per kilogram of body weight of the mammal. More preferably, the dosage will vary from about 1 μg to about 5 mg per kilogram of body weight of the mammal. For example, in some embodiments, the dosage will vary from about 0.005 mg to about 5 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, and so forth.

In certain embodiments, administration of a composition of the present disclosure may be performed by single administration or boosted by multiple administrations.

In certain embodiments, the disclosure includes a method comprising administering a combination of LNP compositions described herein. In certain embodiments, the combination has an additive effect, wherein the overall effect of the administering the combination is approximately equal to the sum of the effects of administering each LNP composition. In other embodiments, the combination has a synergistic effect, wherein the overall effect of administering the combination is greater than the sum of the effects of administering each LNP composition.

In some aspects of the disclosure, the method provides for delivery of compositions for gene editing or genetic manipulation to a target cell (e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell. cartilage cell, brain cell, white blood cell, hematopoietic stem cell, and so forth) of a subject to treat or prevent a disease or disorder (e.g., an immune cell, stem cell, bone cell, blood cell, fat cell, endothelial cell, cancer cell, tissue cell, nerve cell, epithelial cell, connective tissue cell, and/or muscle cell, such as a heart cell, liver cell, spleen cell, lung cell, kidney cell, podocytes, skin cell, cartilage cell, brain cell, white blood cell, hematopoietic stem cell, and so forth).

In another aspect, the therapeutic compounds or compositions of the disclosure may be administered prophylactically (i.e., to prevent disease or disorder, such as a liver disease or disorder, pulmonary disease or disorder, spleen disease or disorder, renal disease or disorder, heart disease or disorder, cardiovascular disease or disorder, brain disease or disorder, neurological disease or disorder, cancer, bone disease or disorder, bone marrow disease or disorder, skin disease or disorder, connective tissue disease or disorder, pancreatic disease or disorder, muscle disease or disorder, lymph node disease or disorder, blood disease or disorder, or any combination thereof) or therapeutically (i.e., to treat disease or disorder, such as a liver disease or disorder, pulmonary disease or disorder, spleen disease or disorder, renal disease or disorder, heart disease or disorder, cardiovascular disease or disorder, brain disease or disorder, neurological disease or disorder, cancer, bone disease or disorder, bone marrow disease or disorder, skin disease or disorder, connective tissue disease or disorder, pancreatic disease or disorder, muscle disease or disorder, lymph node disease or disorder, blood disease or disorder, or any combination thereof) to subjects suffering from or at risk of (or susceptible to) developing the disease or disorder (e.g., a liver disease or disorder, pulmonary disease or disorder, spleen disease or disorder, renal disease or disorder, heart disease or disorder, cardiovascular disease or disorder, brain disease or disorder, neurological disease or disorder, cancer, bone disease or disorder, bone marrow disease or disorder, skin disease or disorder, connective tissue disease or disorder, pancreatic disease or disorder, muscle disease or disorder, lymph node disease or disorder, blood disease or disorder, or any combination thereof). Such subjects may be identified using standard clinical methods.

In the context of the present disclosure, prophylactic administration occurs prior to the manifestation of overt clinical symptoms of disease or disorder (e.g., a liver disease or disorder, pulmonary disease or disorder, spleen disease or disorder, renal disease or disorder, heart disease or disorder, cardiovascular disease or disorder, brain disease or disorder, neurological disease or disorder, cancer, bone disease or disorder, bone marrow disease or disorder, skin disease or disorder, connective tissue disease or disorder, pancreatic disease or disorder, muscle disease or disorder, lymph node disease or disorder, blood disease or disorder, or any combination thereof), such that the disease or disorder is prevented or alternatively delayed in its progression. In the context of the field of medicine, the term “prevent” encompasses any activity which reduces the burden of mortality or morbidity from a disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The composition of the disclosure can be useful in combination with therapeutic, anti-cancer, and/or radiotherapeutic agents. Thus, the present disclosure provides a combination of the present LNP with therapeutic, anti-cancer, and/or radiotherapeutic agents for simultaneous, separate, or sequential administration. The composition of the disclosure and the other anticancer agent can act additively or synergistically.

The therapeutic agent, anti-cancer agent, and/or radiation therapy can be administered according to therapeutic protocols well known in the art. It will be apparent to those skilled in the art that the administration of the therapeutic agent, anti-cancer agent, and/or radiation therapy can be varied depending on the disease being treated and the known effects of the anti-cancer agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., anti-neoplastic agent or radiation) on the patient, and in view of the observed responses of the disease to the administered therapeutic agents, and observed adverse effects.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

Pharmaceutical compositions that are useful in the methods of the disclosure may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 99.99% (w/w) active ingredient.

In addition to the active ingredient, a pharmaceutical composition of the disclosure may further comprise one or more additional pharmaceutically active agents.

Controlled- or sustained-release formulations of a pharmaceutical composition of the disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e. powder or granular) form for reconstitution with a suitable vehicle (e.g. sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the disclosure may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self propelling solvent/powder dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the disclosure are known in the art and described, for example in Remington's Pharmaceutical Sciences (1985, Genaro, ed., Mack Publishing Co., Easton, PA), which is incorporated herein by reference.

### EXAMPLES

Various embodiments of the present application can be better understood by reference to the following Examples which are offered by way of illustration. The scope of the present application is not limited to the Examples given herein.

### Example 1: Biodegradable Lipidoids and Lipid Nanoparticles Facilitated Systemic mRNA Delivery In Vivo

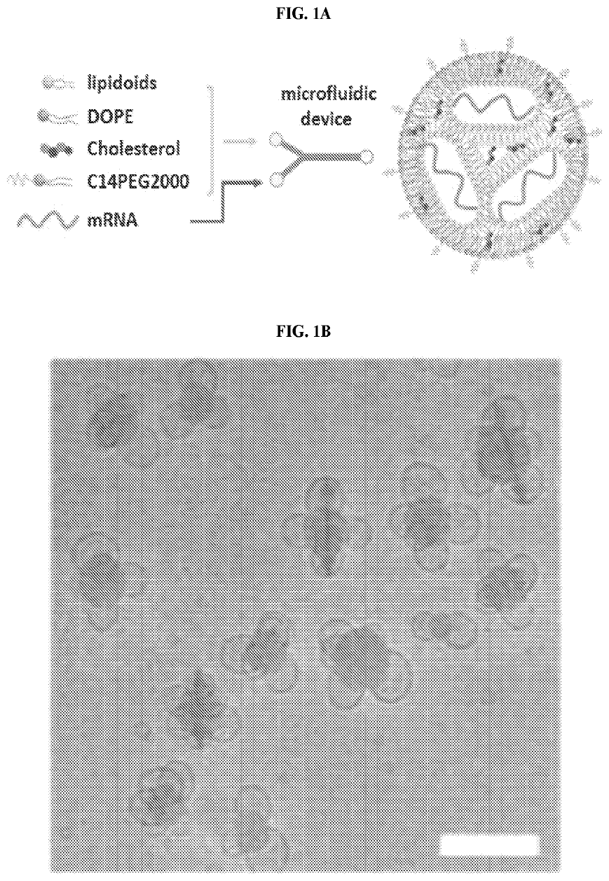

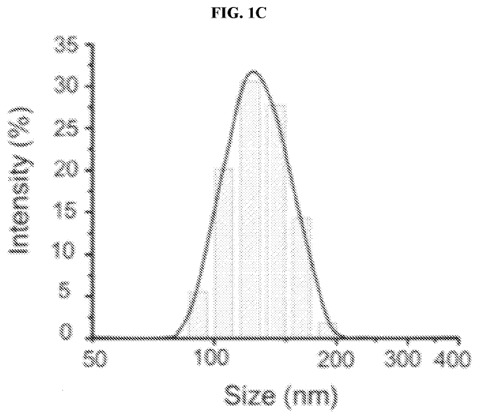

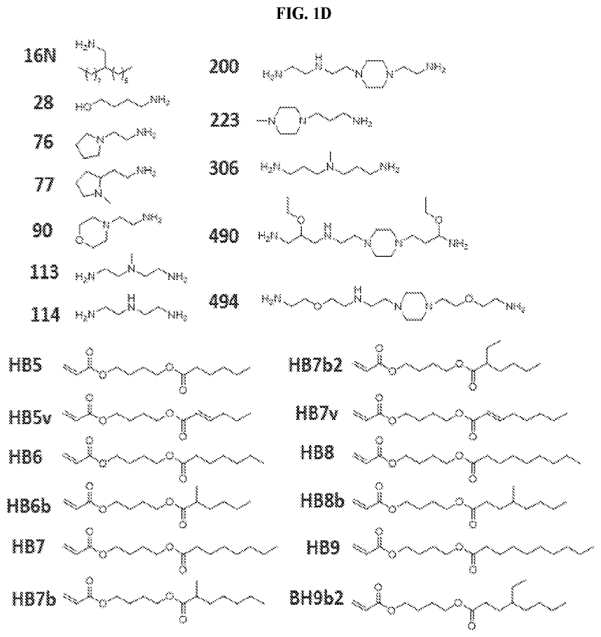

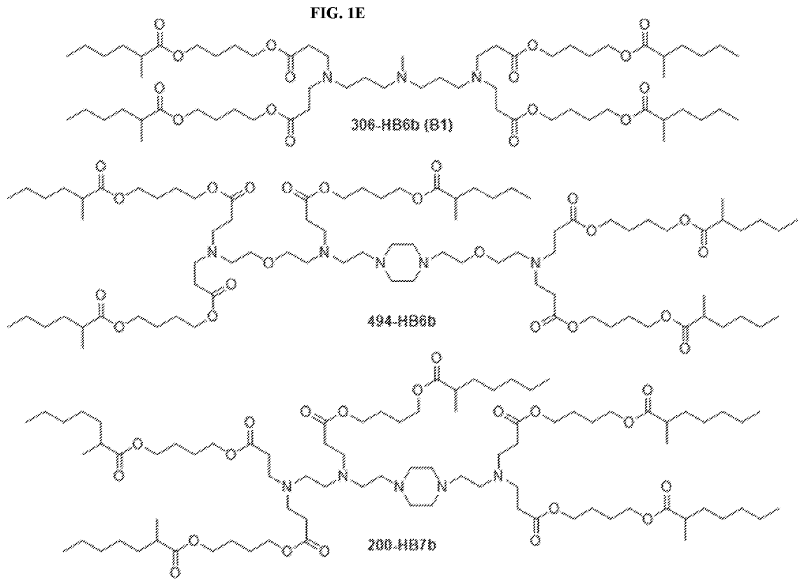

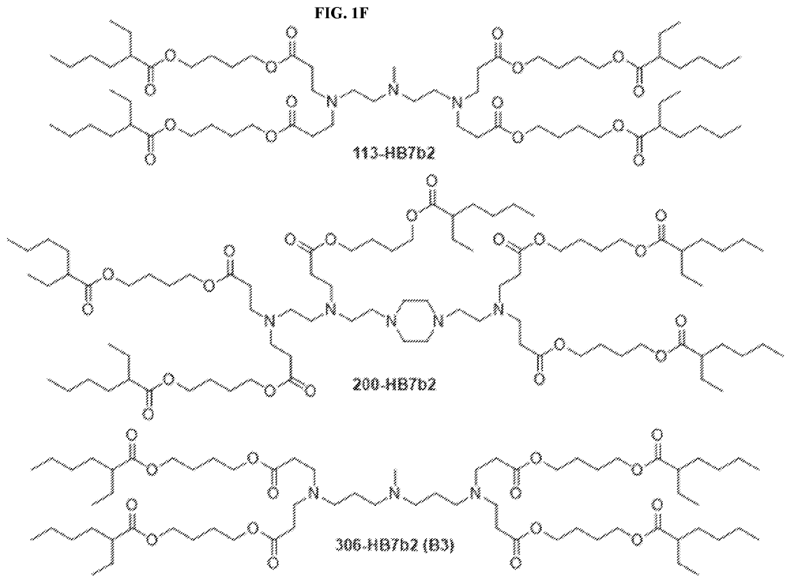

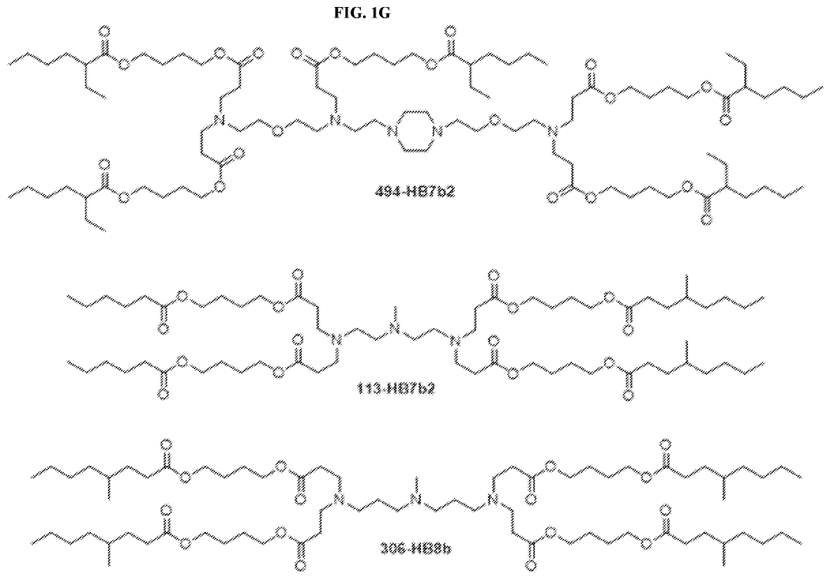

To develop novel lipid nanoparticle (LNP) delivery systems with both high delivery efficacy and low toxicity, the present studies sought to identify degradable LNPs that enable more potent mRNA expression in vivo than the benchmark, without causing off-target toxicities. As shown in FIG. TA, biodegradable lipid nanoparticles (BLNPs) were formulated via microfluidic device with biodegradable ionizable lipids, helper lipid (DOPE), cholesterol, and PEG-lipid (C14PEG2000). The present study utilized 12 amine cores and 12 biodegradable tails to synthesize 144 biodegradable lipidoids (FIGS. 1D-1G). The resulting BLNPs were analyzed with Cryogenic transmission electron microscopy (cryo-TEM) for their size, and structural analysis demonstrated that the obtained BLNPs possessed a flower-like morphology (FIGS. 1B-1C).

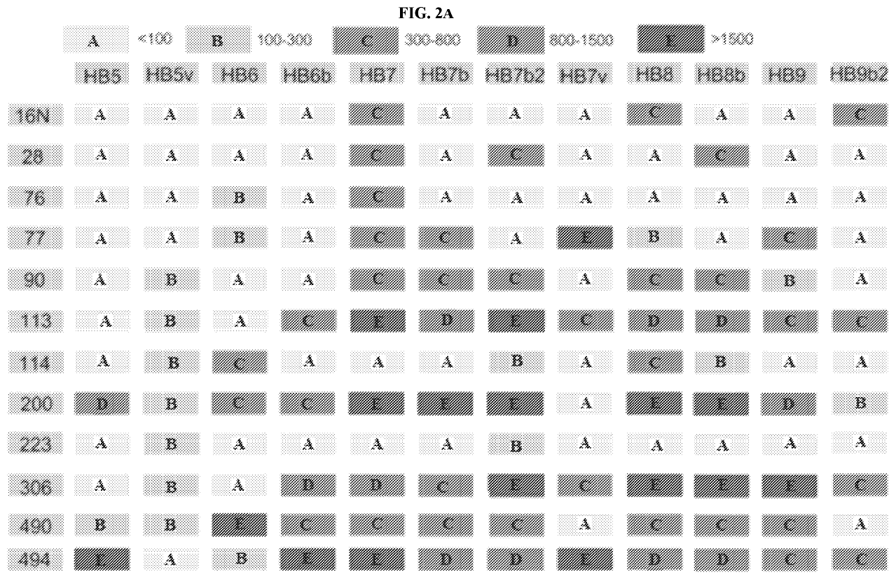

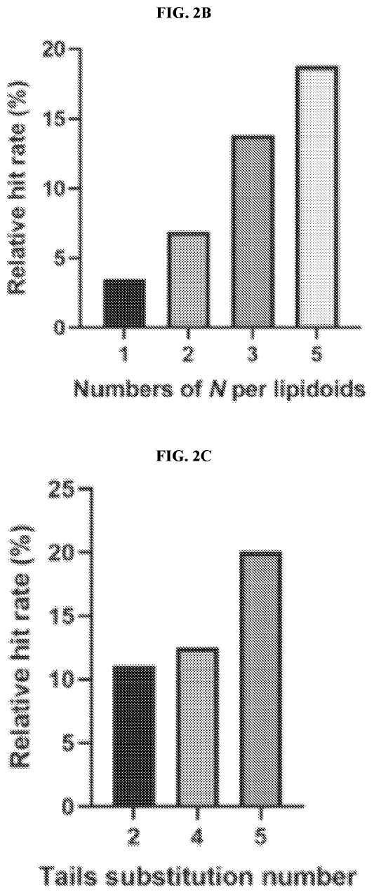

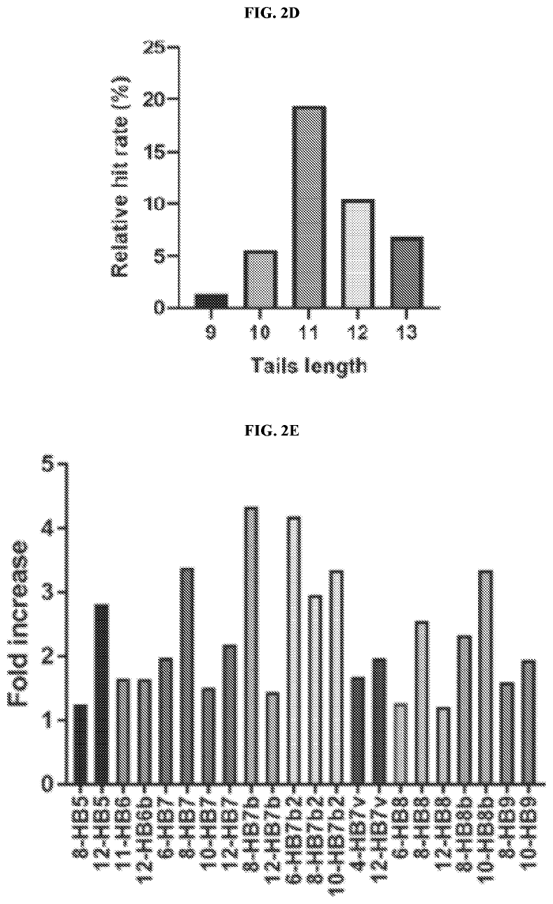

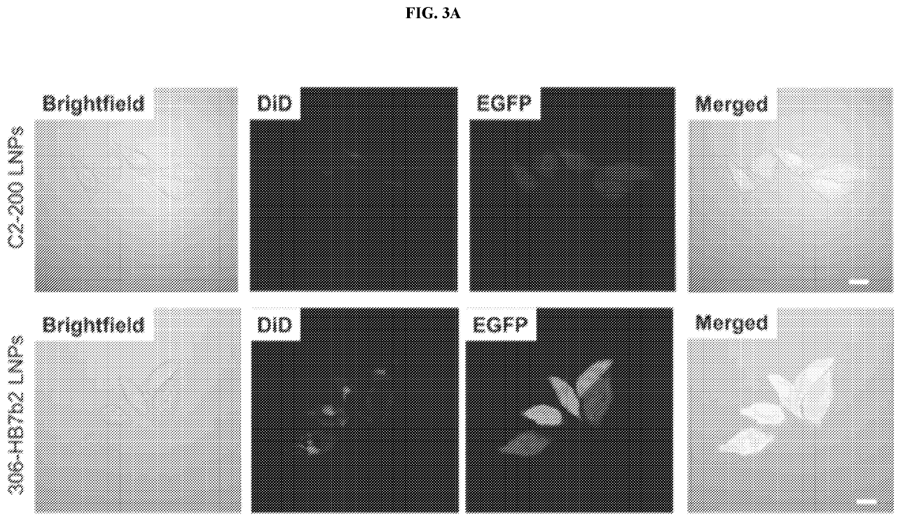

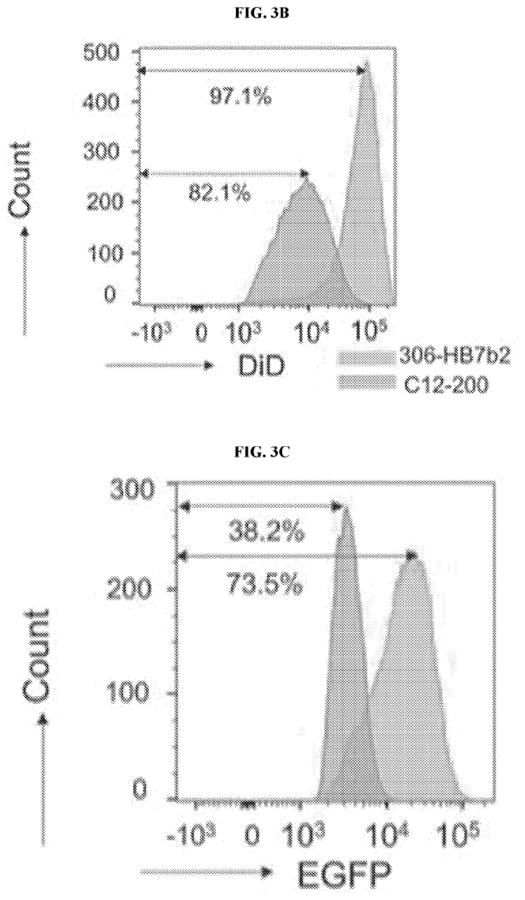

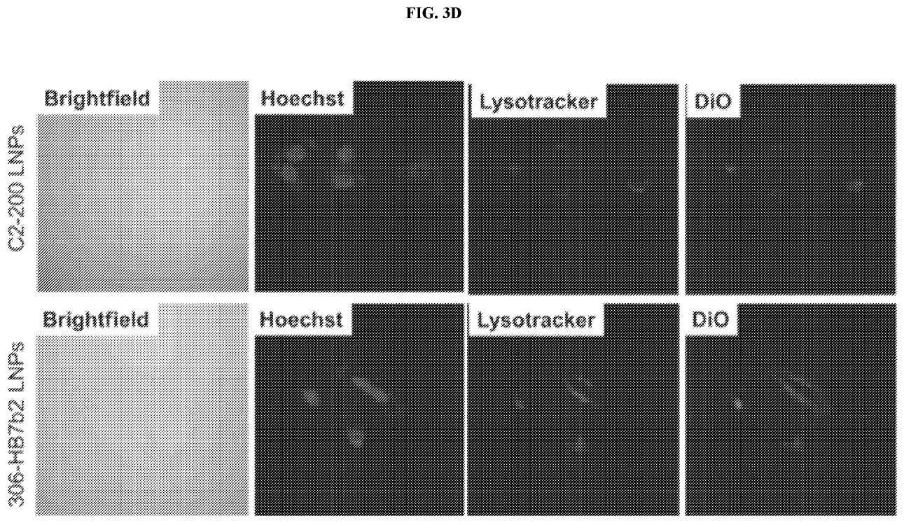

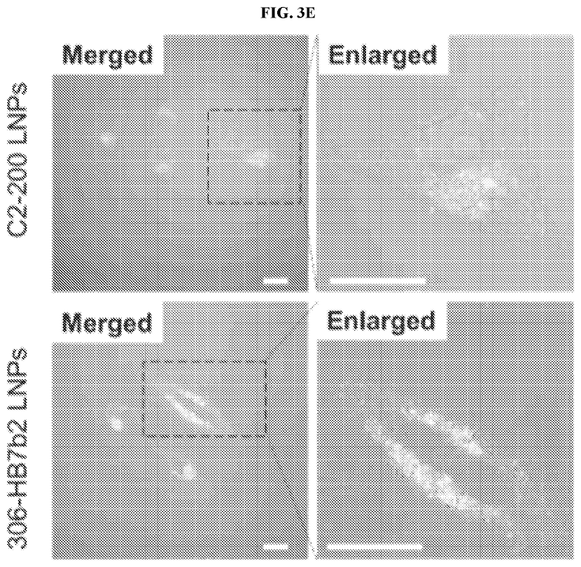

Structure-activity relationships of BLNPs were also accessed for luciferase mRNA delivery in vitro. As shown in FIGS. 2A-2E, heat map of luciferase expression following treatment of Hela cells with BLNPs was counted for hit rate calculation. BLNPs having higher luciferase transfection efficiency were chosen for comparison. More specifically, as shown in FIGS. 3A-3E, the present studies also evaluated nanoparticle uptake, EGFP mRNA transfection, and endosomal escape facilitated by 306-Hb7b2 BLNPs as compared to the benchmark C12-200 in vitro. Samples were incubated for 3 h before imaging and DiD fluorescence dye was used to label LNPs at a concentration of 0.2%. Biodegradable 306-HB7b2 LNPs showed higher cellular uptake and stronger EGFP expression (FIG. 3A). 306-HB7b2 also exhibited much higher DiD intensity than C12-200 (FIG. 3B) and exhibited much higher EGFP transfection efficiency than C12-200 (FIG. 3C).

To evaluate endosomal escape capabilities, Hela cells were treated with 0.5 μg/mL luciferase mRNA encapsulated in LNPs as indicated for 3 h. DiO was used to label the LNPs at a concentration of 1%. Lysotracker was used to stain the endosome for 1 h, while Hoechst dye was used to stain the nucleus for 5 min. Samples and dye markers were washed off before imaging. As shown in FIGS. 3D-3E, 306-HB7b2 LNPs treated cells displayed weaker overlapping of green and red colors than C12-200 LNPs, demonstrating enhanced endosomal escape capability.

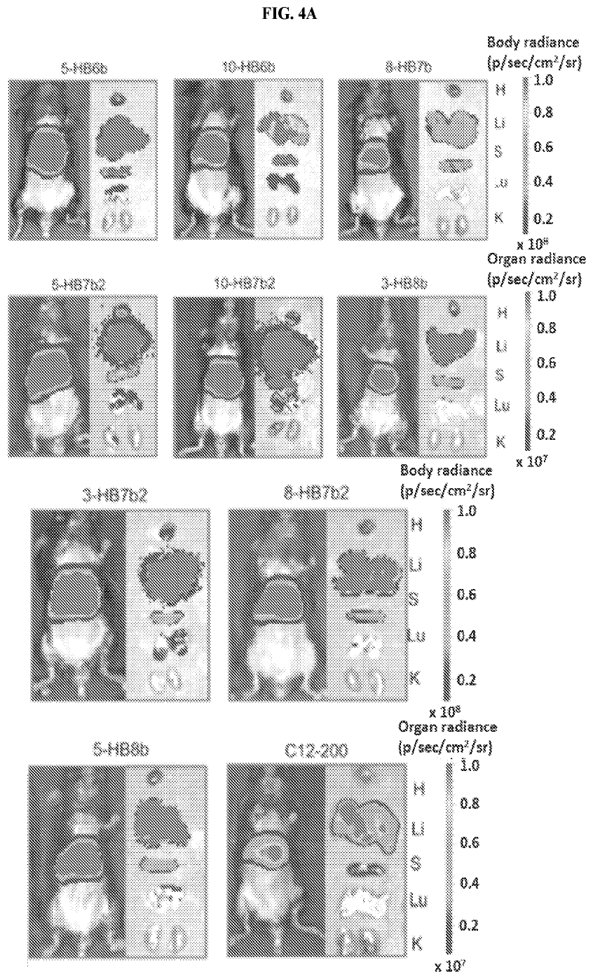

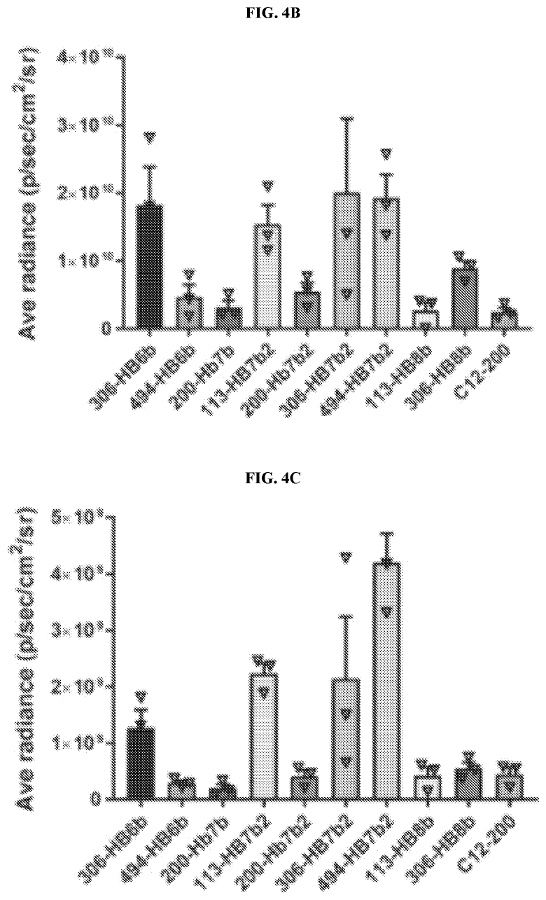

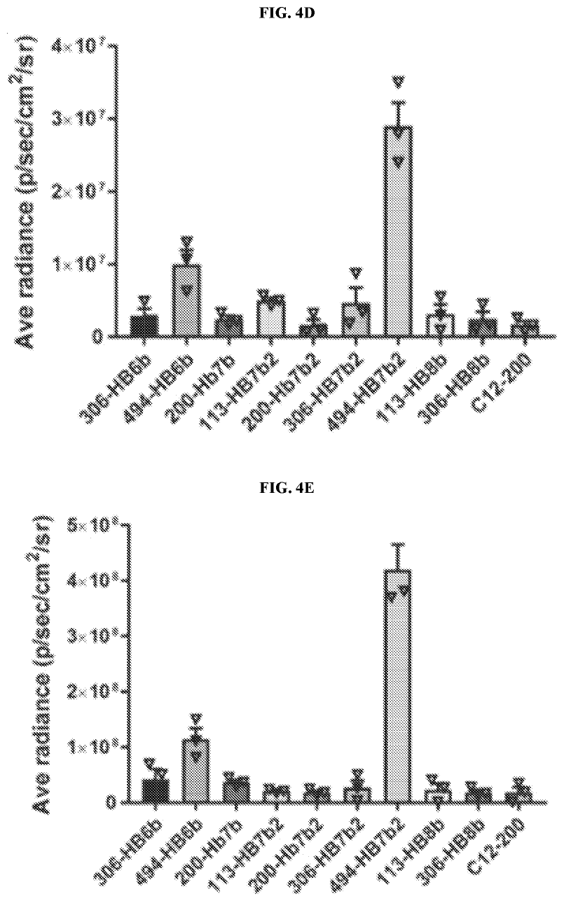

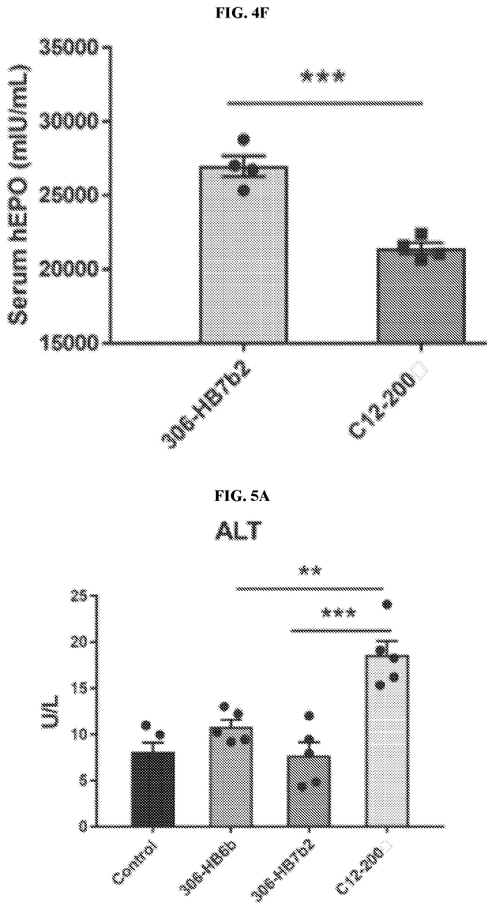

Subsequent structure-activity studies revealed that biodegradable lipid structure demonstrated significantly higher in vivo efficacy than the benchmark C12-200 (FIGS. 4A-4F). More specifically, bioluminescence images of whole bodies and various organs were recorded 12 h after i.v. injection of LNPs into C57BL/6 mice. Representative 9 BLNPs (FIGS. 1E-1G) showed higher mRNA transfection in vivo compared with C12-200 in heart, liver, spleen, lung, and kidney (FIGS. 4A-4F).

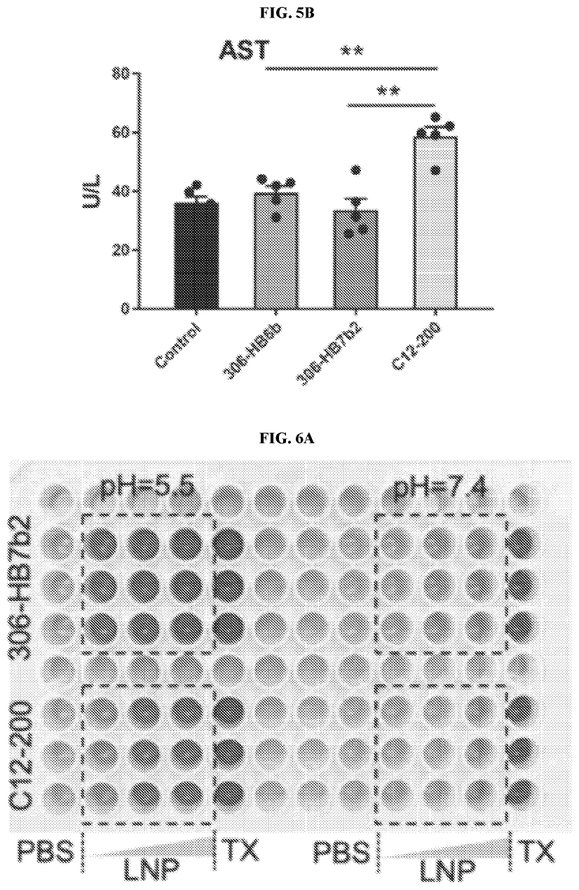

Liver toxicity of BLNPs was assessed using a liver toxicity assay after injection of LNPs encapsulating luciferase-encoding mRNA (FIGS. 5A-5B). Alanine transaminase (ALT) quantification (± standard deviation) was used for control and compared to two representative BLNPs (306-HB6b and 306-HB7b2 LNPs) and the benchmark C12-200 LNPs (FIG. 5A). In a separate study, aspartate transaminase (AST) quantification (±standard deviation) was for control and compared to two representative BLNPs (306-HB6b and 306-HB7b2 LNPs) and the benchmark C12-200 LNPs (FIG. 5B). C57BL/6J mice were dosed with 1.0 mg/kg luciferase mRNA LNPs, and liver enzymes were quantified 12 h after injection. Two representative BLNPs showed much lower liver toxicity than benchmark.

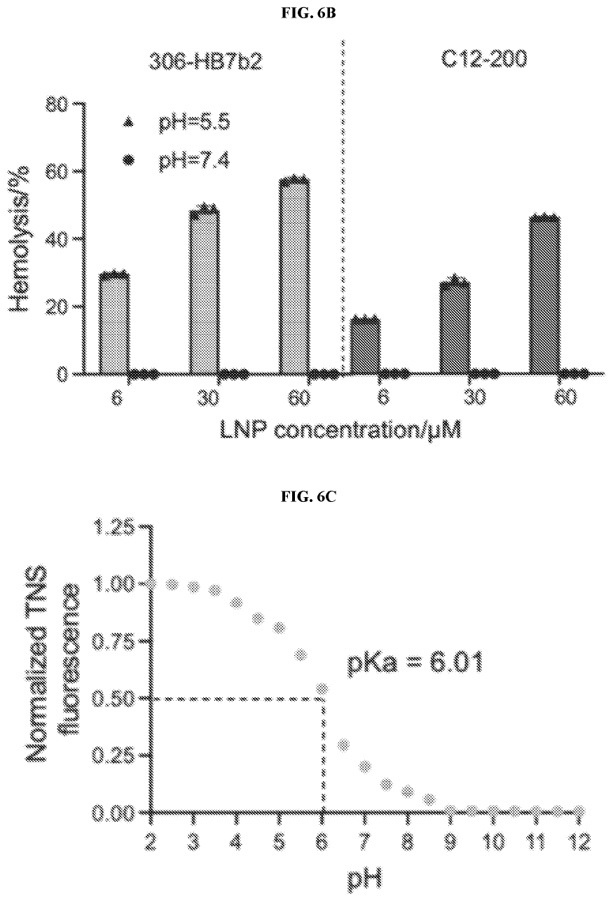

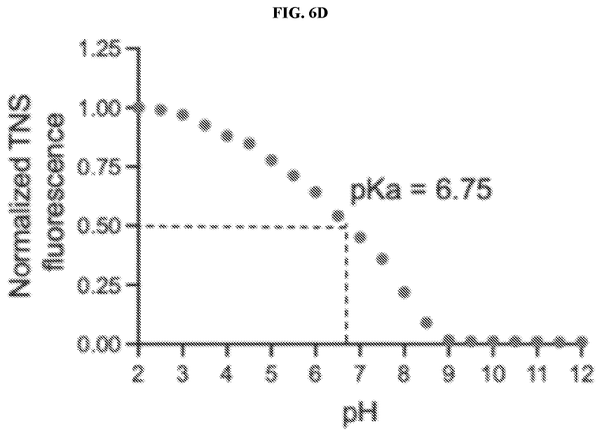

The hemolysis performance of exemplary LNP 306-HB7b2 and benchmark C12-200 LNP were further compared at pH=5.5 and pH=7.4 (FIGS. 6A-6D). Dose-dependent hemolysis of LNP treated groups were observed, with exemplary LNP 306-HB7b2 demonstrating superior hemolysis ability. Thus, in one aspect, these results demonstrate a greater membrane fusion performance for endosomal escape for exemplary LNP 306-HB7b2, as compared to benchmark C12-200 LNP.

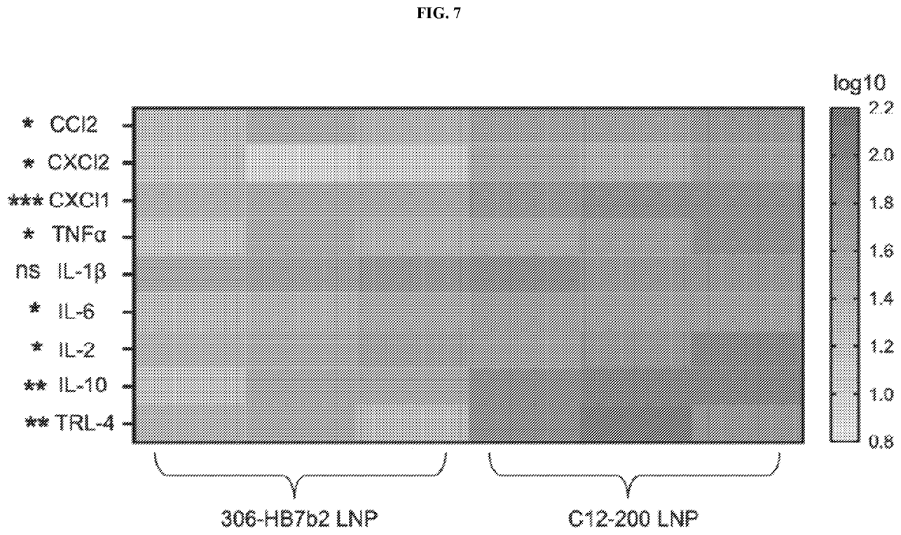

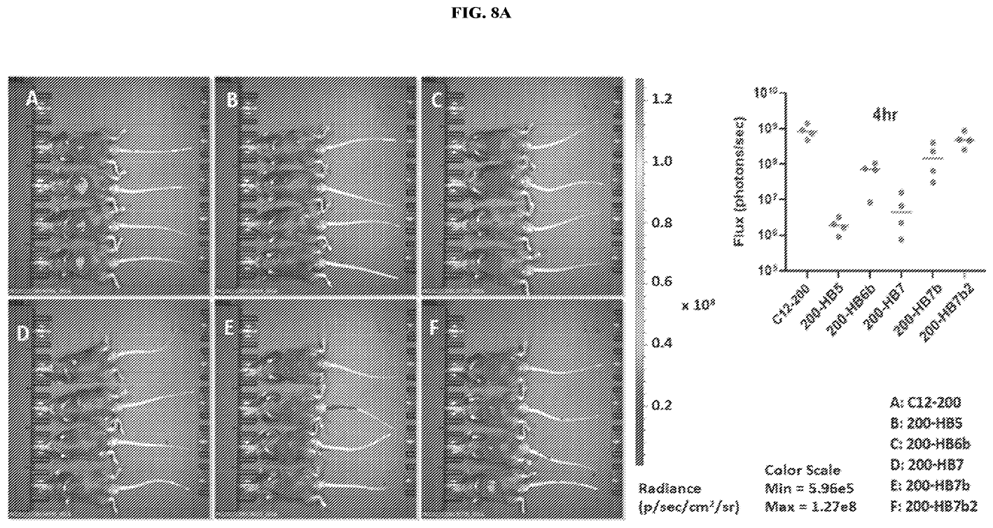

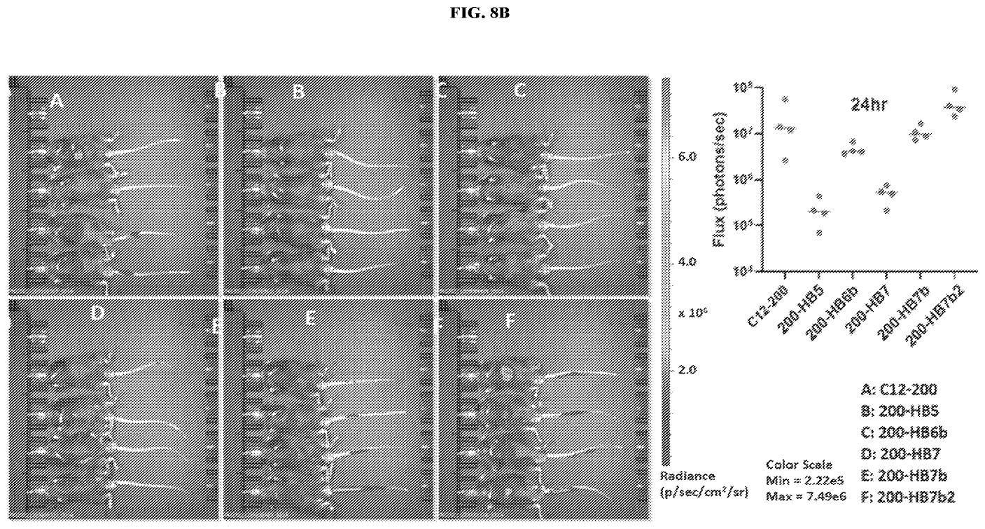

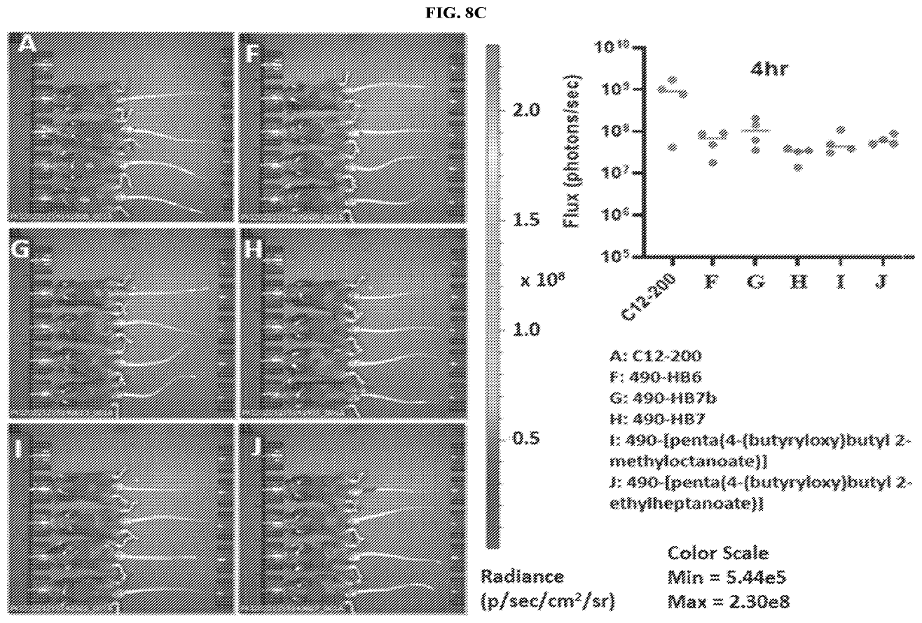

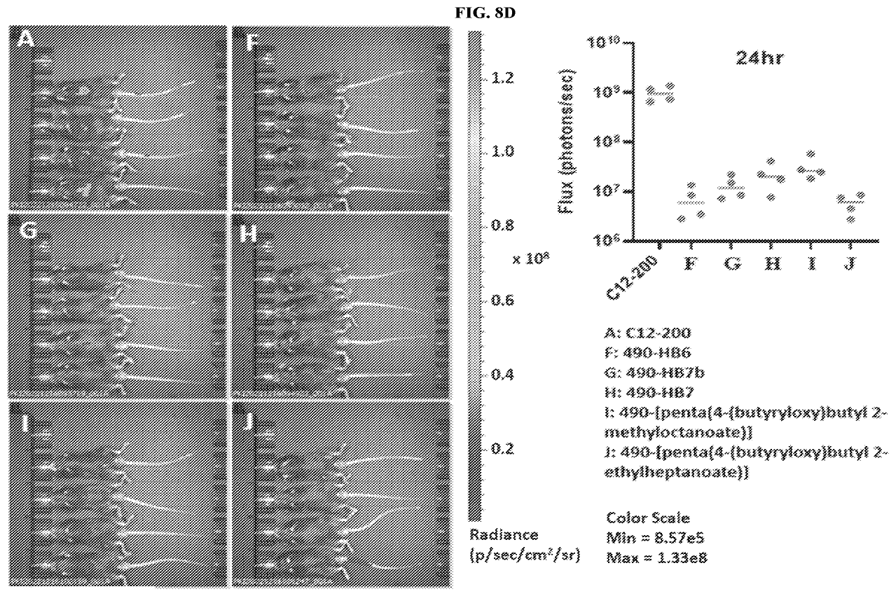

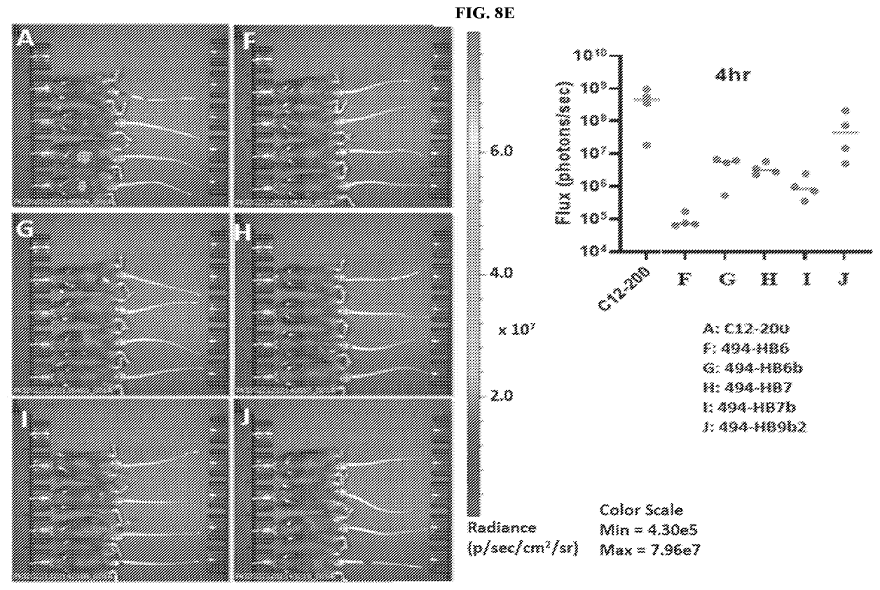

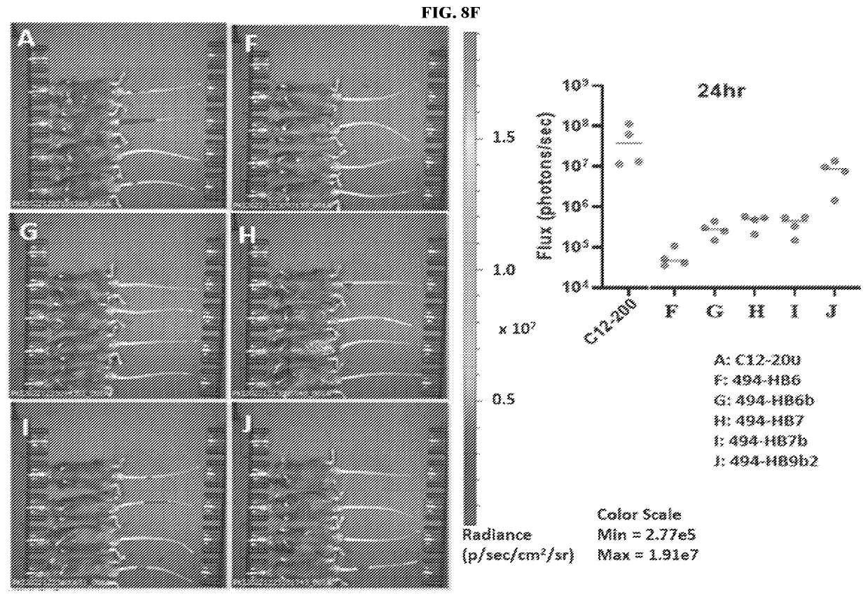

Further, immunogenicity of exemplary LNP 306-HB7b2 and benchmark C12-200 LNP were compared by measuring cytokine and chemokine levels in serum of mice administered the LNPs (FIG. 7). In the studies described herein, lower immunogenicity was observed with exemplary LNP 306-HB7b2 as compared to benchmark C12-200 LNP, indicating a capacity for more efficient mRNA delivery.

In summary, the studies described herein provide novel biodegradable lipidoids and BLNP compositions that demonstrated high delivery efficacy and low toxicity for targeted delivery to various organs, tissues, and cell types of interest, such as heart, liver, spleen, lung, and kidney.

### ENUMERATED EMBODIMENTS

The following exemplary embodiments are provided, the numbering of which is not to be construed as designating levels of importance:

Embodiment 1 provides a compound of Formula (I), or a salt, solvate, stereoisomer, isotopologue, or derivative thereof:

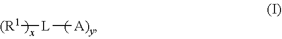

wherein:
   each occurrence of A is independently

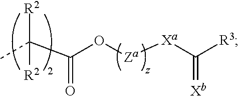

L is an amine linker selected from the group consisting of optionally substituted aminoalkyl linker, optionally substituted diaminoalkyl linker, optionally substituted triaminoalkyl linker, optionally substituted tetraaminoalkyl linker, optionally substituted pentaaminoalkyl linker, optionally substituted polyaminoalkyl linker, optionally substituted aminocycloalkyl linker, optionally substituted diaminocycloalkyl linker, optionally substituted triaminocycloalkyl linker, optionally substituted tetraaminocycloalkyl linker, optionally substituted pentaaminocycloalkyl linker, and optionally substituted polyaminocycloalkyl linker;
each occurrence of X^{a} and X^{b} is independently selected from the group consisting of —O—, —S—, —N(R⁶)_{y′}—, —P(R⁶)_{y′}—;
each occurrence of Z^{a} is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, and optionally substituted C₂-C₈ heterocyloalkylenyl;
each occurrence of R¹, R², and R³, if present, is independently selected from the group consisting of hydrogen, halogen, optionally substituted C₁-C₁₂ alkyl, optionally substituted C₃-C₁₂ cycloalkyl, optionally substituted —Y(R⁶)_{z′}(R⁷)_{z″}—(C₃-C₁₂ cycloalkyl), optionally substituted C₂-C₁₂ heterocycloalkyl, optionally substituted-(R⁶)_{z′}(R⁷)_{z″}-(C₂-C₁₂ heterocycloalkyl), optionally substituted C₂-C₁₂ alkenyl, optionally substituted C₅-C₁₂ cycloalkenyl, optionally substituted —Y(R⁶)_{z′}(R⁷)_{z″}—(C₅-C₁₂ cycloalkenyl), optionally substituted C₂-C₁₂ alkynyl, optionally substituted C₅-C₁₂ cycloalkynyl, optionally substituted —Y(R⁶)_{z′}(R⁷)_{z″}—(C₅-C₁₂ cycloalkynyl), optionally substituted C₆-C₁₀ aryl, optionally substituted —Y(R⁶)_{z′}(R⁷)_{z″}—(C₆-C₁₀ aryl), optionally substituted C₂-C₁₂ heteroaryl, optionally substituted —Y(R⁶)_{z′}(R⁷)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁶)_{z′}(R⁷)_{z″}-ester, —Y(R⁶)_{z′}(R⁷)_{z″}, —NO₂, —CN, sulfoxy, sulfonate, sulfate, sulfite, and sulfide, or two geminal R² groups can combine to form ═O or ═S;
each occurrence of R⁶ and R⁷ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, ═S, sulfoxy, sulfonate, sulfate, sulfite, and sulfide, or two geminal R⁶ and R⁷ groups can combine to form ═O or ═S;
each occurrence of Y is independently selected from the group consisting of C, O, N, S, P, and Si; and
x is an integer from 0 to 10;
y is an integer from 1 to 10;
each occurrence of z is an integer from 0 to 20;
each occurrence of y′, z′ and z″ is independently an integer represented by 0, 1, or 2.

Embodiment 2 provides the compound of Embodiment 1, wherein each N atom in L is independently substituted with 0-2 instances of A.

Embodiment 3 provides the compound of Embodiment 1 or 2, wherein x is 0.

Embodiment 4 provides the compound of any one of Embodiments 1-3, wherein L is selected from the group consisting of:

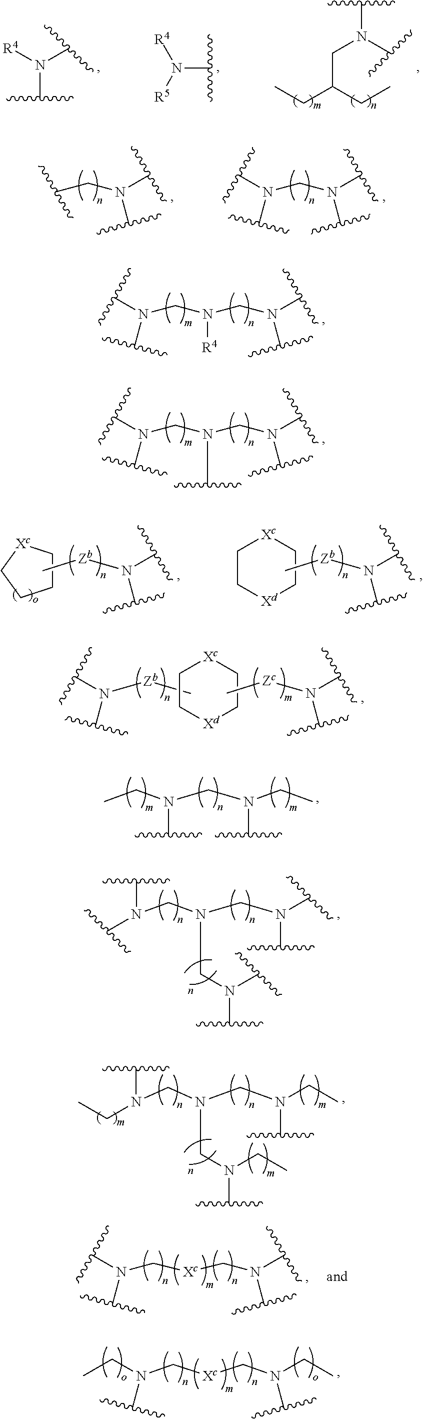

wherein:
   R⁴ and R⁵, if present, are each independently selected from the group consisting of hydrogen, halogen, optionally substituted C₁-C₁₂ alkyl, optionally substituted C₃-C₁₂ cycloalkyl, optionally substituted —Y(R⁶)_{z′}(R⁷)_{z″}—(C₃-C₁₂ cycloalkyl), optionally substituted C₂-C₁₂ heterocycloalkyl, optionally substituted-(R⁶)_{z′}(R⁷)_{z″}—(C₂-C₁₂ heterocycloalkyl), optionally substituted C₂-C₁₂ alkenyl, optionally substituted C₅-C₁₂ cycloalkenyl, optionally substituted —Y(R⁶)_{z′}(R⁷)_{z″}—(C₅-C₁₂ cycloalkenyl), optionally substituted C₂-C₁₂ alkynyl, optionally substituted C₅-C₁₂ cycloalkynyl, optionally substituted —Y(R⁶)_{z′}(R⁷)_{z″}—(C₅-C₁₂ cycloalkynyl), optionally substituted C₆-C₁₀ aryl, optionally substituted —Y(R⁶)_{z′}(R⁷)_{z″}—(C₆-C₁₀ aryl), optionally substituted C₂-C₁₂ heteroaryl, optionally substituted —Y(R⁶)_{z′}—(R⁷)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁶)_{z′}(R^{T})_{z′}-ester, —Y(R⁶)_{z′}(R⁷)_{z′}, —NO₂, —CN, sulfoxy, sulfonate, sulfate, sulfite, and sulfide;
   each occurrence of X^{c} and X^{d} is independently selected from the group consisting of —O—, —S—, —N(R⁶)_{y′}—, —P(R⁶)_{y′}—;
   each occurrence of Z^{b} and Z° is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, and optionally substituted C₂-C₈ heterocyloalkylenyl;
   each occurrence of m, n, and o is independently an integer from 0 to 10; and
   each occurrence of

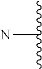 [SMILES: *N]

indicates a bond between a N atom and A or R¹, if present.

Embodiment 5 provides the compound of Embodiment 4, wherein L is selected from the group consisting of

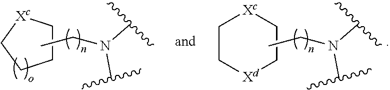

Embodiment 6 provides the compound of any one of Embodiments 1-5, wherein L is selected from the group consisting of:

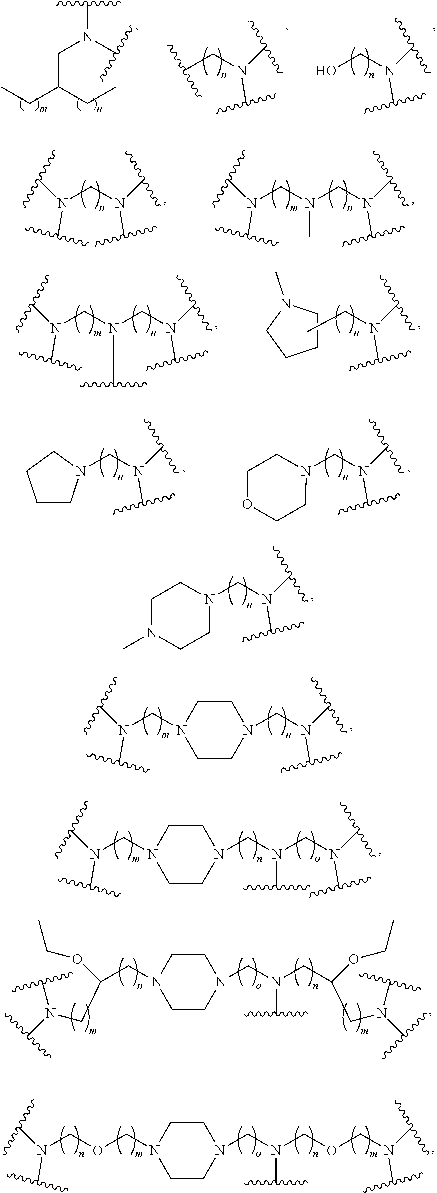

wherein:
   each occurrence of m, n, and o is independently an integer from 0 to 10; and
   each occurrence of

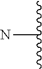 [SMILES: *N]

indicates a bond between a N atom and A or R¹, if present.

Embodiment 7 provides the compound of Embodiment 1, which is selected from the group consisting of:

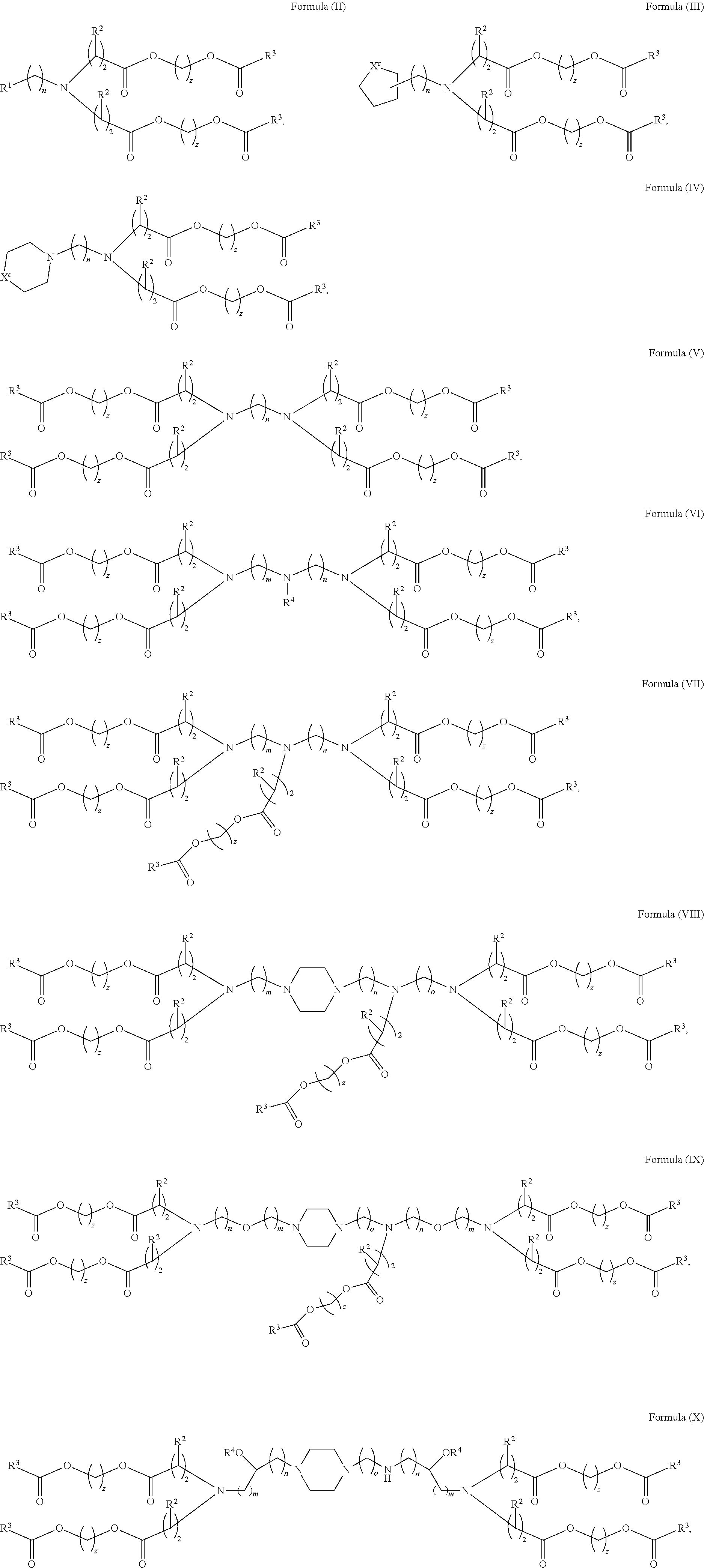

wherein:
   each occurrence of X^{c} is independently selected from the group consisting of —O—, —S—, —N(R⁶)_{y′}—, and —P(R⁶)_{y′}—;
   each occurrence of R⁴ is independently selected from the group consisting of hydrogen, halogen, optionally substituted C₁-C₁₂ alkyl, optionally substituted C₃-C₁₂ cycloalkyl, optionally substituted —Y(R⁶)_{z′}(R⁷)_{z″}—(C₃-C₁₂ cycloalkyl), optionally substituted C₂-C₁₂ heterocycloalkyl, optionally substituted-(R⁶)_{z′}(R⁷)_{z″}—(C₂-C₁₂ heterocycloalkyl), optionally substituted C₂-C₁₂ alkenyl, optionally substituted C₅-C₁₂ cycloalkenyl, optionally substituted —Y(R⁶)_{z′}(R⁷)_{z″}—(C₅-C₁₂ cycloalkenyl), optionally substituted C₂-C₁₂ alkynyl, optionally substituted C₅-C₁₂ cycloalkynyl, optionally substituted —Y(R⁶)_{z′}(R⁷)_{z″}—(C₅-C₁₂ cycloalkynyl), optionally substituted C₆-C₁₀ aryl, optionally substituted —Y(R⁶)_{z′}(R⁷)_{z″}—(C₆-C₁₀ aryl), optionally substituted C₂-C₁₂ heteroaryl, optionally substituted —Y(R⁶)_{z′}—(R⁷)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁶)_{z′}(R⁷)_{z″}-ester, —Y(R⁶)_{z′}(R⁷)_{z′}, —NO₂, —CN, sulfoxy, sulfonate, sulfate, sulfite, and sulfide; and
   each occurrence of m, n, and o is independently an integer from 0 to 10.

Embodiment 8 provides the compound of Embodiment 7, wherein at least one of the following applies:
(a) each occurrence of R¹, R³, and R⁴ is alkyl or substituted alkyl;
(b) each occurrence of R² is hydrogen;
(c) each occurrence of X^{c} is O or N(R⁶)_{y′}, wherein R⁶ is alkyl and y′ is 1;
(d) each occurrence of m and o is an integer represented by 2;
(e) each occurrence of n is an integer represented by 2 or 3; and
(f) each occurrence of z is an integer represented by 4.

Embodiment 9 provides the compound of any one of Embodiments 1-6, wherein at least one of the following applies:
(a) each occurrence of R² is H;
(b) each occurrence of Z^{a} is —CH₂—;
(c) each occurrence of z is an integer represented by 4;
(d) each occurrence of X^{a} is 0;
(e) each occurrence of X^{b} is 0;
(f) x is an integer represented by 0;
(g) R¹ is absent;
(h) y is an integer represented by 2, 3, 4, or 5; and
(i) each occurrence of R³ is independently selected from the group consisting of n-pentyl, n-hexyl, n-heptyl, n-octyl, n-nonyl, 1-pentenyl, 1-heptenyl, 1-methylpentyl, 1-methylhexyl, 1-methylheptyl, 1-ethylpentyl, 2-ethylhexyl, and 3-methylheptyl, optionally wherein each occurrence of R³ is identical.

Embodiment 10 provides the compound of Embodiment 1, which is selected from the group consisting of:

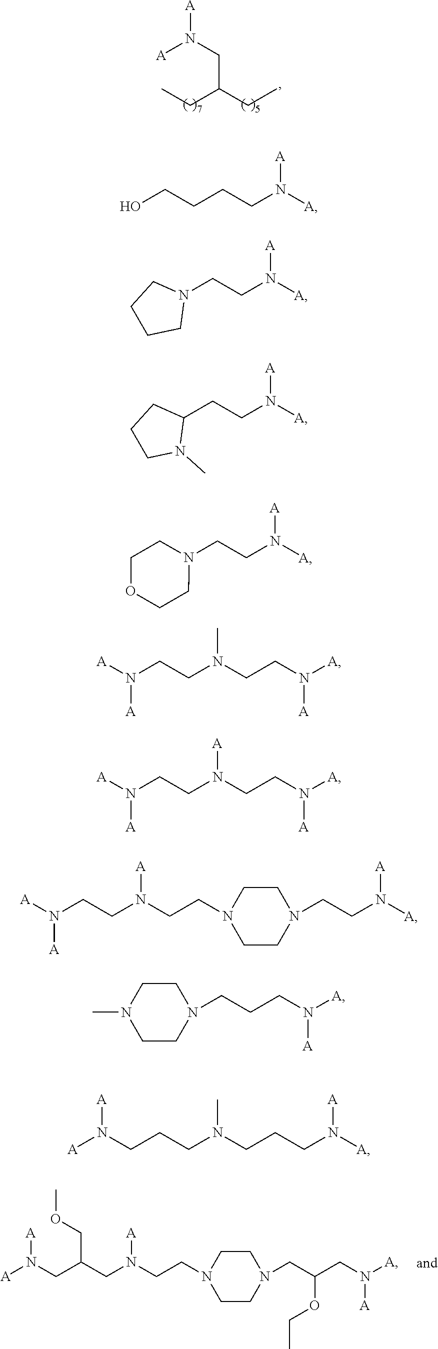

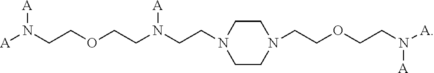

Embodiment 11 provides the compound of any one of Embodiments 1-6 and 9-10, wherein each occurrence of A is independently selected from the group consisting of

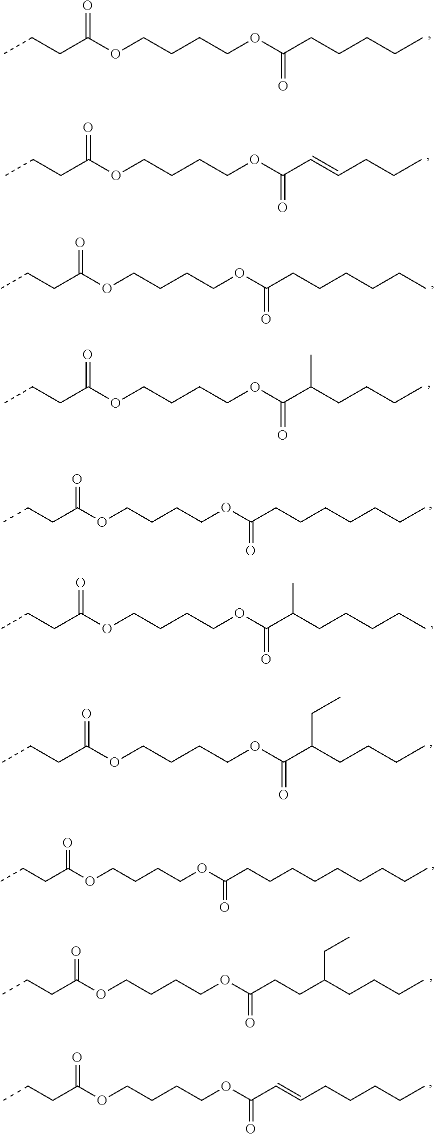

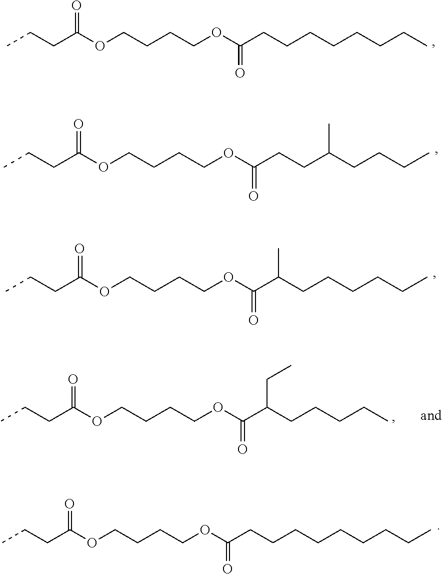

Embodiment 12 provides the compound of any one of Embodiments 1-11, wherein the compound of Formula (I) is selected from the group consisting of:

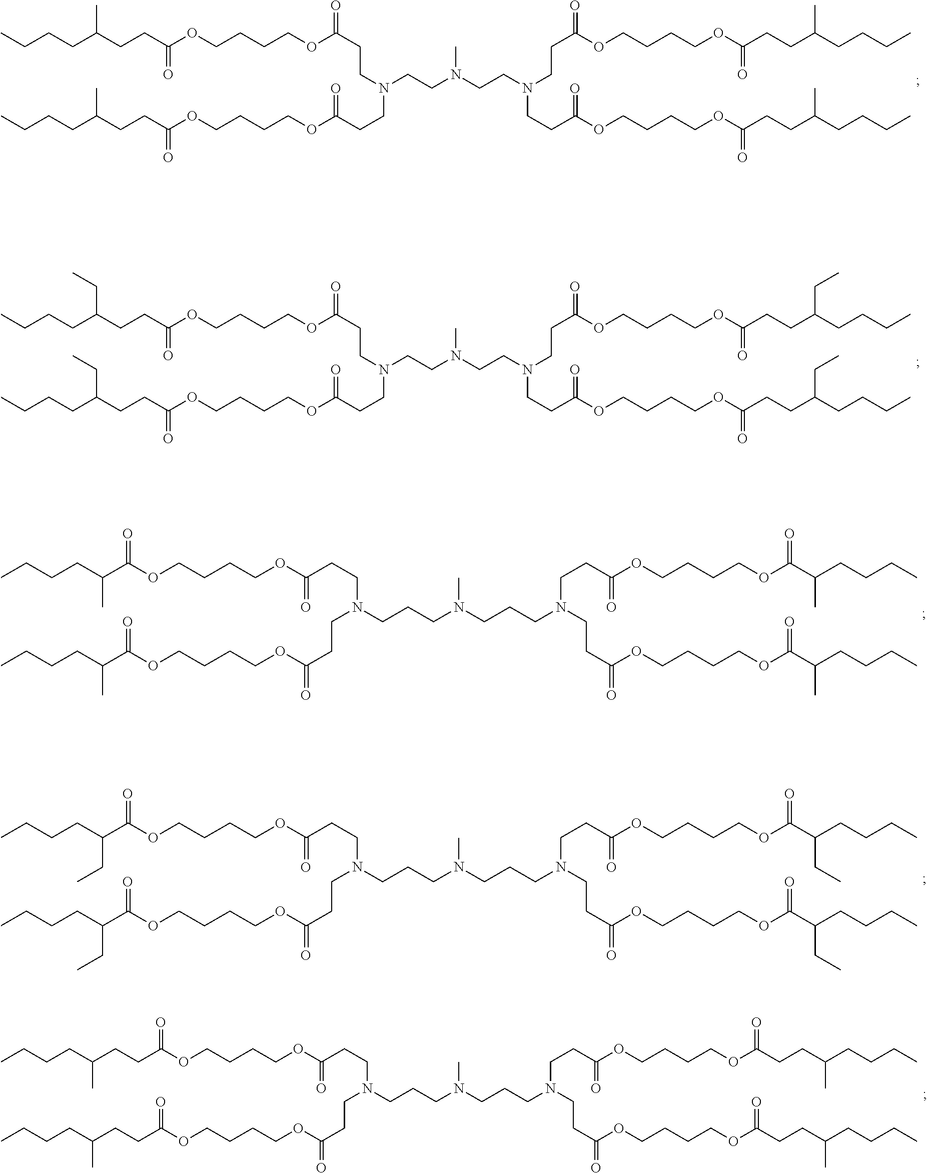

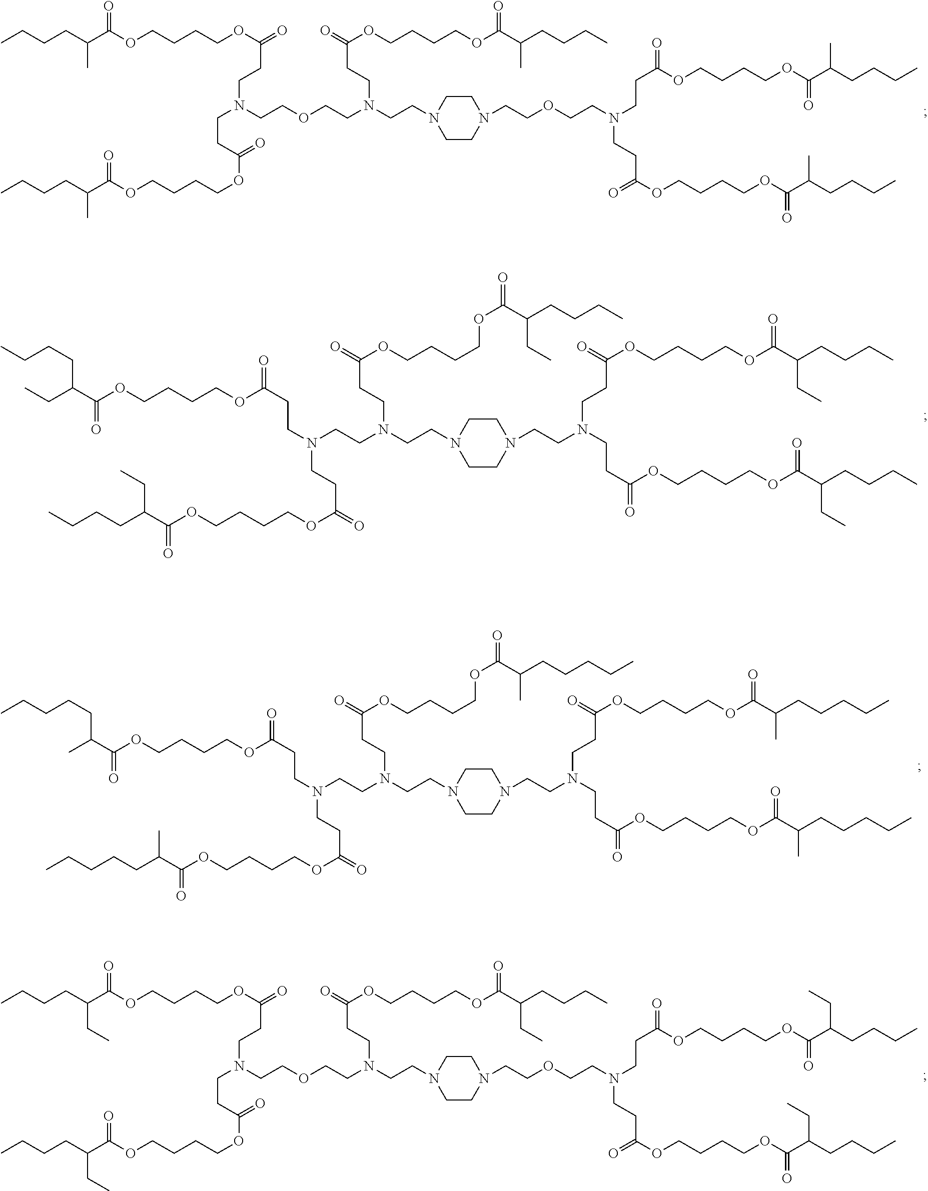

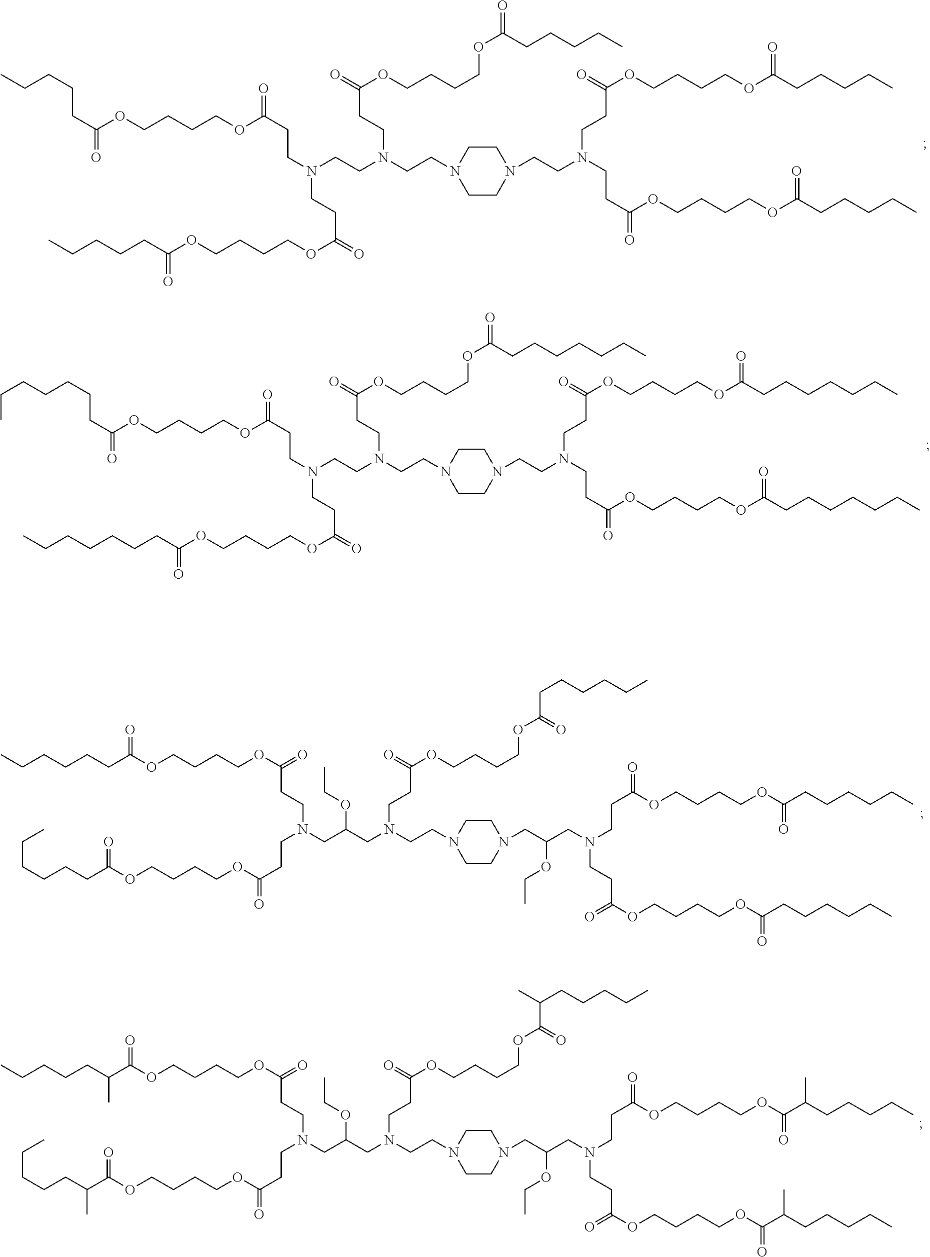

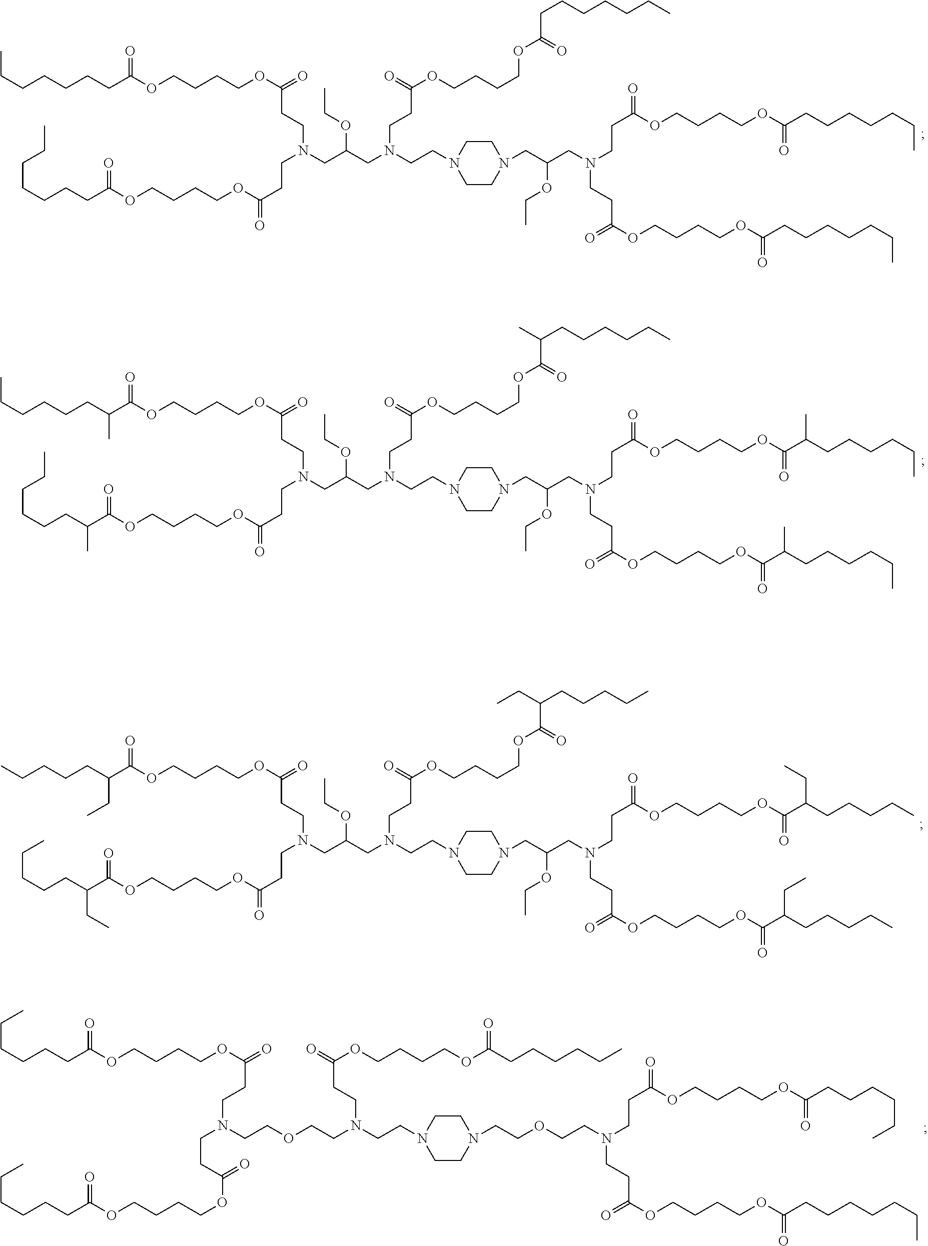

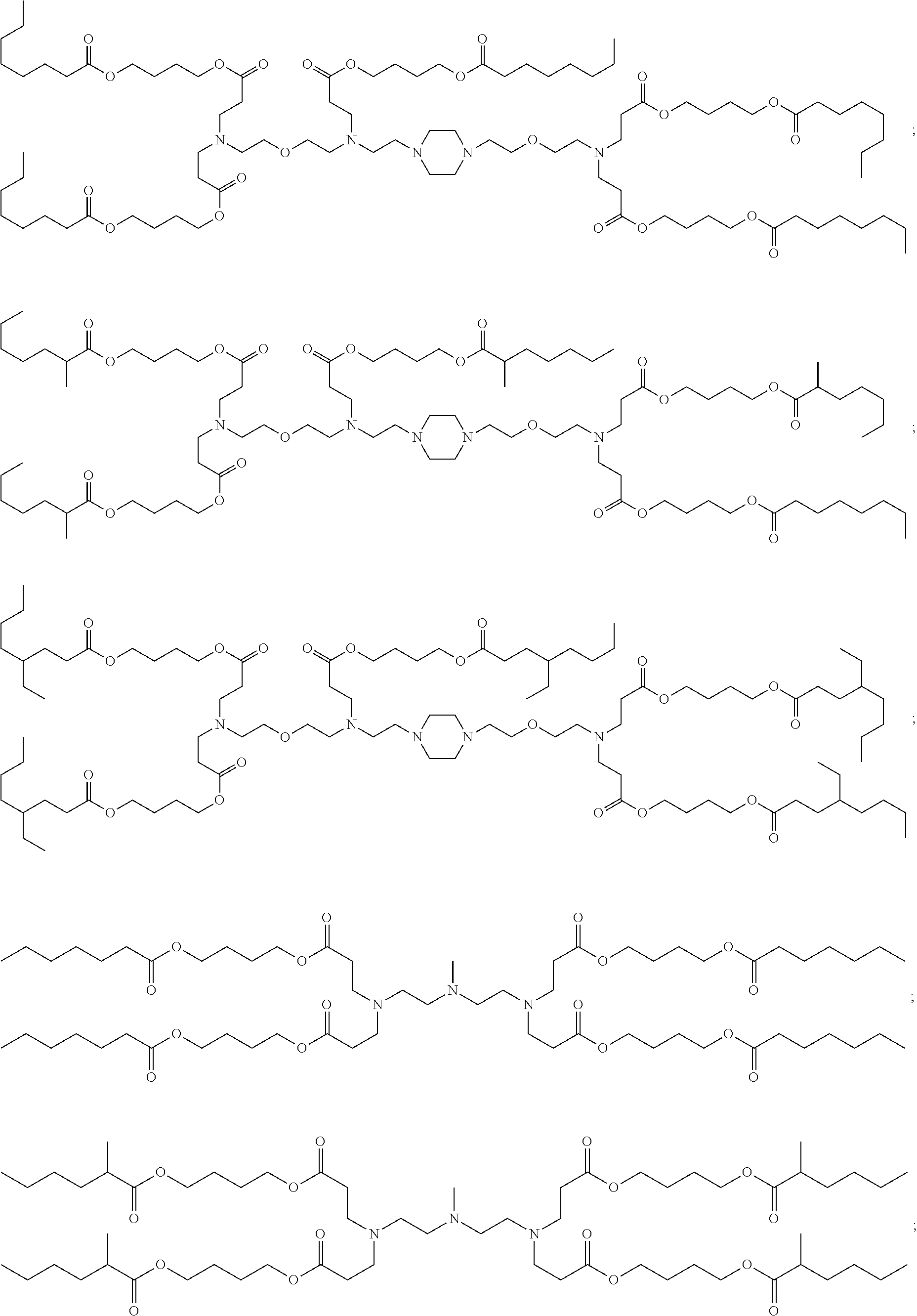

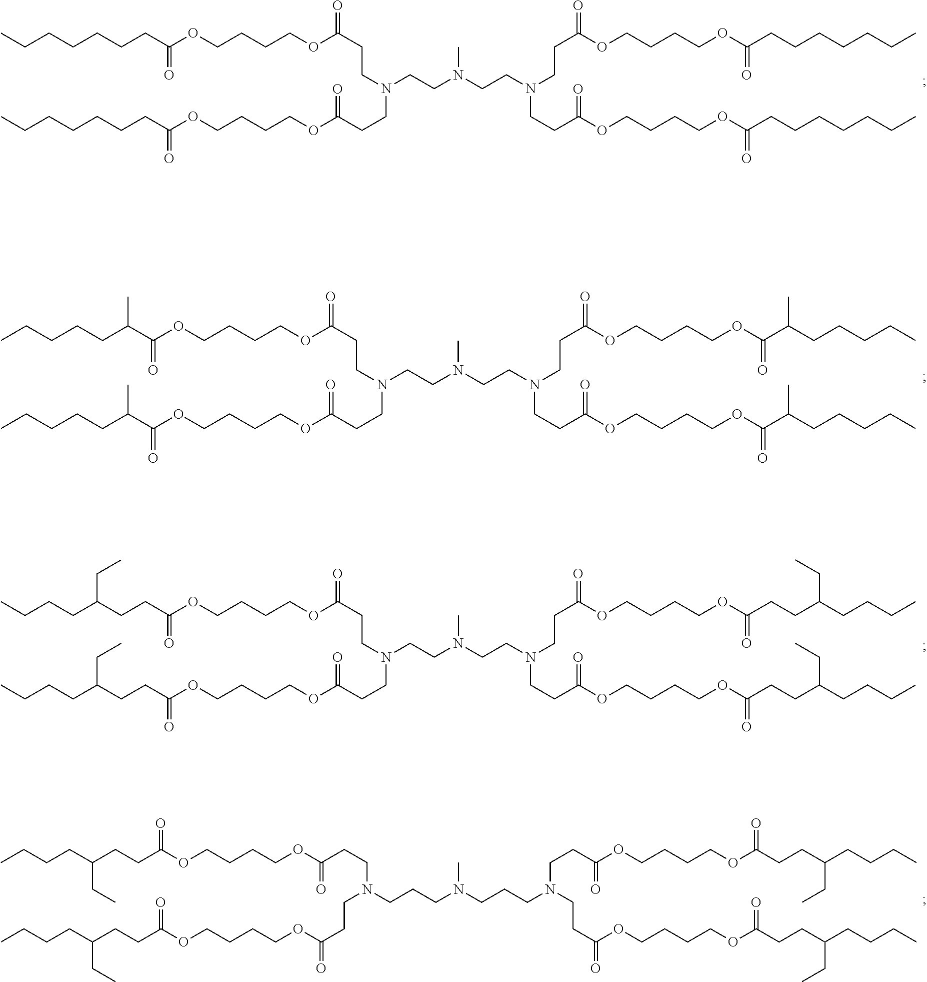

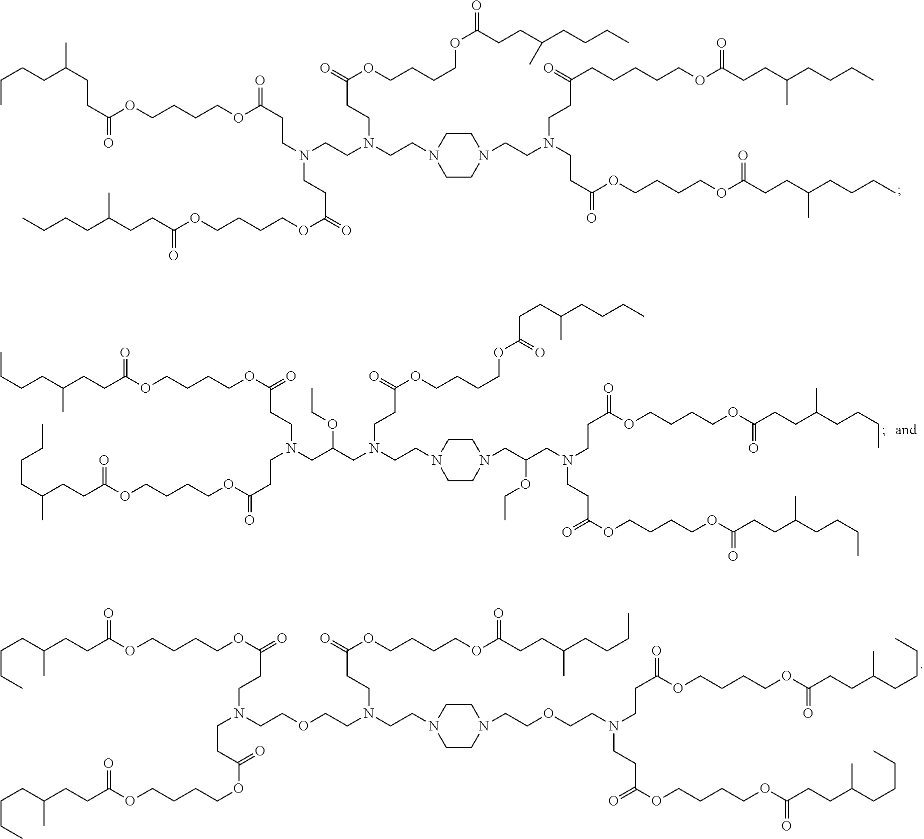

Embodiment 13 provides the compound of any one of Embodiments 1-12, wherein the compound of Formula (I) is selected from the group consisting of

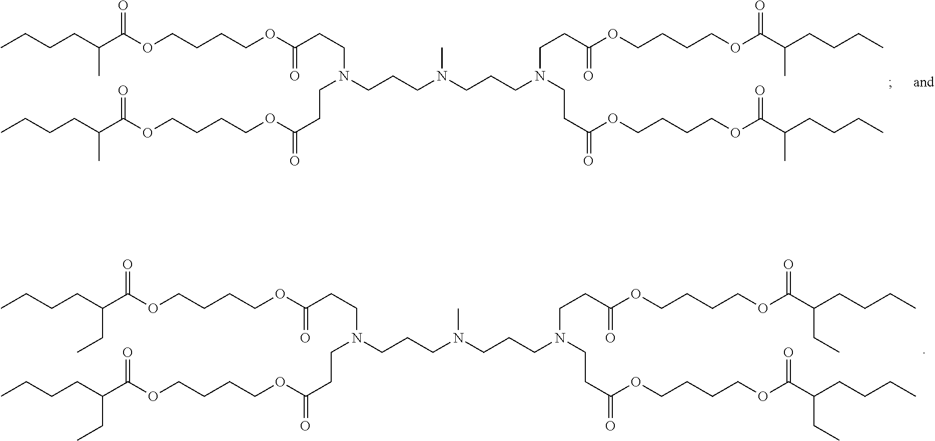

Embodiment 14 provides a biodegradable lipid nanoparticle (LNP) comprising:
(a) at least one compound of any one of Embodiments 1-13;
(b) at least one neutral phospholipid, wherein the neutral phospholipid is present in a concentration range of about 5 mol % to about 45 mol %;
(c) at least one cholesterol lipid, wherein the total cholesterol lipid is in a concentration range of about 5 mol % to about 55 mol %; and
(d) at least one polyethylene glycol (PEG) or PEG-conjugated lipid, wherein the PEG or PEG-conjugated lipid is in a concentration range of about 0.5 mol % to about 12.5 mol %.

Embodiment 15 provides the biodegradable LNP of Embodiment 14, wherein the at least one neutral phospholipid comprises at least one selected from the group consisting of dioleoyl-phosphatidylethanolamine (DOPE), dioleoylphosphatidylcholine (DOPC), distearoylphosphatidylcholine (DSPC), distearoyl-phosphatidylethanolamine (DSPE), 16-O-dimethyl PE, 18-1-trans PE, 1-stearioyl-2-oleoyl-phosphatidyethanol amine (SOPE), stearoyloleoylphosphatidylcholine (SOPC), N-(2,3-dioleoyloxy)propyl)-N,N,N-trimethylammonium chloride (DOTAP), and any combination thereof.

Embodiment 16 provides the biodegradable LNP of Embodiment 14 or 15, wherein the at least one cholesterol lipid comprises at least one selected from the group consisting of a cholesterol, cholesterol derivate, and any combination thereof.

Embodiment 17 provides the biodegradable LNP of any one of Embodiments 14-16, wherein the at least one PEG or PEG-conjugated lipid comprises at least one selected from the group consisting of 1,2-dimyristoyl-sn-glycero-3-phosphoethanolamine-N-[methoxy(polyethylene glycol)-2000](C14-PEG2000), C12-PEG2000, C12-PEG490, 1,2-dimyristoyl-rac-gly cero-3-methoxypolyethylene glycol-2000 (DMG-PEG2000), 1,2-distearoyl-sn-glycero-3-phosphoethanolamine-N-[amino(polyethylene glycol)-2000](DSPE-PEG 2000 amine), and any combination thereof.

Embodiment 18 provides the biodegradable LNP of any one of Embodiments 14-17, wherein the molar ratio of (a):(b):(c):(d) is about 1-80:5-45:5-55:0.5-12.5.

Embodiment 19 provides the biodegradable LNP of any one of Embodiments 14-18, wherein the molar ratio of (a):(b):(c):(d) is about 25-45:5-20:40-55:1-2.5.

Embodiment 20 provides the biodegradable LNP of any one of Embodiments 14-19, wherein the molar ratio of (a):(b):(c):(d) is selected from the group consisting of about 30:16:46.5:2.5, 31:16:46.5:2.5, 32:16:46.5:2.5, 33:16:46.5:2.5, 34:16:46.5:2.5, and 35:16:46.5:2.5.

Embodiment 21 provides the biodegradable LNP of any one of Embodiments 14-20, wherein the biodegradable LNP has a diameter of between about 50 nm to about 500 nm.

Embodiment 22 provides the biodegradable LNP of any one of Embodiments 14-21, wherein the biodegradable LNP has a diameter of between about 50 nm to about 160 nm.

Embodiment 23 provides the biodegradable LNP of any one of Embodiments 14-22, wherein the biodegradable LNP selectively binds to at least one target cell of interest.

Embodiment 24 provides the biodegradable LNP of any one of Embodiments 14-23, wherein the at least one target cell of interest is selected from the group consisting of an immune cell, stem cell, bone cell, blood cell, fat cell, endothelial cell, cancer cell, tissue cell, nerve cell, epithelial cell, connective tissue cell, muscle cell, and any combination thereof.

Embodiment 25 provides the biodegradable LNP of any one of Embodiments 14-24, wherein the at least one target cell of interest is selected from the group consisting of a brain cell, neuron, neuroglial cell, heart cell, liver cell, spleen cell, lung cell, kidney cell, podocytes, skin cell, keratinocyte, melanocyte, merkel cell, langerhans cell, cartilage cell, chondrocyte, pancreatic cell, skeletal muscle cell, cardiac muscle cell, smooth muscle cell, bone cell, osteoblast, osteoclast, osteocyte, lining cell, bone marrow cell, lymph node cell, white blood cell, granulocyte, neutrophil, eosinophil, basophil, agranulocyte, monocyte, lymphocyte, red blood cell, erythrocyte, platelet, fragments of megakaryocyte, embryonic stem cell, adult stem cell, mesenchymal stem cell, hematopoietic stem cell, white adipocyte, brown adipocyte, and any combination thereof.

Embodiment 26 provides the biodegradable LNP of any one of Embodiments 14-26, wherein the biodegradable LNP further comprises or encapsulates at least one agent.

Embodiment 27 provides the biodegradable LNP of Embodiment 26, wherein the weight ratio of (a):the at least one agent is between about 1:1 to about 10:1.

Embodiment 28 provides the biodegradable LNP of Embodiment 26 or 27, wherein the agent is selected from the group consisting of a nucleic acid molecule, a small molecule, a protein, an antibody, and any combination thereof.

Embodiment 29 provides the biodegradable LNP of Embodiment 28, wherein the nucleic acid molecule is a DNA molecule or an RNA molecule.

Embodiment 30 provides the biodegradable LNP of Embodiment 28, wherein the nucleic acid molecule is selected from the group consisting of cDNA, mRNA, miRNA, siRNA, sgRNA, modified RNA, antagomir, antisense molecule, targeted nucleic acid, and any combination thereof.

Embodiment 31 provides the biodegradable LNP of any one of Embodiments 14-30, wherein the biodegradable LNP is suitable for delivering a nucleic acid to a liver cell.

Embodiment 32 provides a composition comprising at least one biodegradable LNP of any one of Embodiments 14-31.

Embodiment 33 provides the composition of Embodiment 32, further comprising at least one pharmaceutically acceptable carrier.

Embodiment 34 provides the composition of Embodiment 32 or 33, wherein the composition is suitable for delivering a nucleic acid to a liver cell.

Embodiment 35 provides a method of delivering an agent to a subject in need thereof, the method comprising administering a therapeutically effectively amount of at least one biodegradable LNP of any one of Embodiments 14-31 or a composition comprising the same to the subject.

Embodiment 36 provides the method of Embodiment 35, wherein the agent is selected from the group consisting of a nucleic acid molecule, a small molecule, a protein, an antibody, a therapeutic agent, and any combination thereof.

Embodiment 37 provides the method of Embodiment 36, wherein the nucleic acid molecule is a DNA molecule or an RNA molecule.

Embodiment 38 provides the method of Embodiment 36, wherein the nucleic acid molecule is selected from the group consisting of cDNA, mRNA, miRNA, siRNA, sgRNA, modified RNA, antagomir, antisense molecule, targeted nucleic acid, and any combination thereof.

Embodiment 39 provides the method of Embodiment 35, wherein the biodegradable LNP selectively binds to at least one target cell of interest.

Embodiment 40 provides the method of Embodiment 39, wherein the at least one target cell of interest is selected from the group consisting of an immune cell, stem cell, bone cell, blood cell, fat cell, endothelial cell, cancer cell, tissue cell, nerve cell, epithelial cell, connective tissue cell, muscle cell, and any combination thereof.

Embodiment 41 provides the method of Embodiment 39, wherein the at least one target cell of interest is selected from the group consisting of a brain cell, neuron, neuroglial cell, heart cell, liver cell, spleen cell, lung cell, kidney cell, podocytes, skin cell, keratinocyte, melanocyte, merkel cell, langerhans cell, cartilage cell, chondrocyte, pancreatic cell, skeletal muscle cell, cardiac muscle cell, smooth muscle cell, bone cell, osteoblast, osteoclast, osteocyte, lining cell, bone marrow cell, lymph node cell, white blood cell, granulocyte, neutrophil, eosinophil, basophil, agranulocyte, monocyte, lymphocyte, red blood cell, erythrocyte, platelet, fragments of megakaryocyte, embryonic stem cell, adult stem cell, mesenchymal stem cell, hematopoietic stem cell, white adipocyte, brown adipocyte, and any combination thereof.

Embodiment 42 provides the method of Embodiment 39, wherein the at least one target cell of interest is a liver cell.

Embodiment 43 provides the method of Embodiment 35, wherein the agent is encapsulated within the biodegradable LNP.

Embodiment 44 provides the method of Embodiment 35, wherein the biodegradable LNP or the composition comprising the same is administered intravenously, intramuscularly, intradermally, subcutaneously, intranasally, by inhalation, or any combination thereof.

Embodiment 45 provides a method of delivering an agent to a liver, the method comprising administering a therapeutically effectively amount of at least one biodegradable LNP of any one of Embodiments 14-31 or a composition comprising the same.

Embodiment 46 provides the method of Embodiment 45, wherein the agent is selected from the group consisting of a nucleic acid molecule, a small molecule, a protein, an antibody, a therapeutic agent, and any combination thereof.

Embodiment 47 provides the method of Embodiment 46, wherein the nucleic acid molecule is a DNA molecule or an RNA molecule.

Embodiment 48 provides the method of Embodiment 46, wherein the nucleic acid molecule is selected from the group consisting of cDNA, mRNA, miRNA, siRNA, sgRNA, modified RNA, antagomir, antisense molecule, targeted nucleic acid, and any combination thereof.

Embodiment 49 provides the method of Embodiment 45, wherein the biodegradable LNP selectively binds to at least one liver cell.

Embodiment 50 provides the method of Embodiment 45, wherein the agent is encapsulated within the biodegradable LNP.

Embodiment 51 provides the method of Embodiment 45, wherein the biodegradable LNP or the composition comprising the same is administered intravenously, intramuscularly, intradermally, subcutaneously, intranasally, by inhalation, or any combination thereof.

Embodiment 52 provides a method of treating, ameliorating, and/or preventing at least one disease or disorder in a subject in need thereof, the method comprising administering a therapeutically effectively amount of at least one biodegradable LNP of any one of Embodiments 14-31 or a composition comprising the same to the subject.

Embodiment 53 provides the method of Embodiment 52, wherein the disease or disorder is selected from the group consisting of liver disease or disorder, pulmonary disease or disorder, spleen disease or disorder, renal disease or disorder, heart disease or disorder, cardiovascular disease or disorder, brain disease or disorder, neurological disease or disorder, cancer, bone disease or disorder, bone marrow disease or disorder, skin disease or disorder, connective tissue disease or disorder, pancreatic disease or disorder, muscle disease or disorder, lymph node disease or disorder, blood disease or disorder, and any combination thereof.

Embodiment 54 provides a method of inducing an immune response in a subject in need thereof, the method comprising administering a therapeutically effectively amount of at least one biodegradable LNP of any one of Embodiments 14-31 or a composition comprising the same to the subject.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A compound of Formula (I), or a salt, solvate, stereoisomer, isotopologue, or derivative thereof: 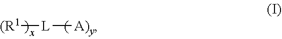
wherein:
each occurrence of A is independently 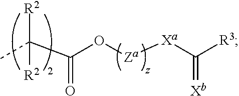
L is an amine linker selected from the group consisting of optionally substituted aminoalkyl linker, optionally substituted diaminoalkyl linker, optionally substituted triaminoalkyl linker, optionally substituted tetraaminoalkyl linker, optionally substituted pentaaminoalkyl linker, optionally substituted polyaminoalkyl linker, optionally substituted aminocycloalkyl linker, optionally substituted diaminocycloalkyl linker, optionally substituted triaminocycloalkyl linker, optionally substituted tetraaminocycloalkyl linker, optionally substituted pentaaminocycloalkyl linker, and optionally substituted polyaminocycloalkyl linker;
each occurrence of X^{a} and X^{b} is independently selected from the group consisting of —O—, —S—, —N(R⁶)_{y′}—, —P(R⁶)_{y′}—;
each occurrence of Z^{a} is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, and optionally substituted C₂-C₈ heterocyloalkylenyl;
each occurrence of R¹, R², and R³, if present, is independently selected from the group consisting of hydrogen, halogen, optionally substituted C₁-C₁₂ alkyl, optionally substituted C₃-C₁₂ cycloalkyl, optionally substituted —Y(R⁶)_{z′}(R⁷)_{z″}—(C₃-C₁₂ cycloalkyl), optionally substituted C₂-C₁₂ heterocycloalkyl, optionally substituted-(R⁶)_{z′}(R⁷)_{z″}—(C₂-C₁₂ heterocycloalkyl), optionally substituted C₂-C₁₂ alkenyl, optionally substituted C₅-C₁₂ cycloalkenyl, optionally substituted —Y(R⁶)_{z′}(R⁷)_{z″}—(C₅-C₁₂ cycloalkenyl), optionally substituted C₂-C₁₂ alkynyl, optionally substituted C₈-C₁₂ cycloalkynyl, optionally substituted —Y(R⁶)_{z′}(R⁷)_{z″}—(C₈-C₁₂ cycloalkynyl), optionally substituted C₆-C₁₀ aryl, optionally substituted —Y(R⁶)_{z′}(R⁷)_{z″}—(C₆-C₁₀ aryl), optionally substituted C₂-C₁₂ heteroaryl, optionally substituted —Y(R⁶)_{z′}(R⁷)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁶)_{z′}(R⁷)_{z″}-ester, —Y(R⁶)_{z′}(R⁷)_{z″}, —NO₂, —CN, sulfoxy, sulfonate, sulfate, sulfite, and sulfide, or two geminal R² groups can combine to form ═O or ═S;
each occurrence of R⁶ and R⁷ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, ═S, sulfoxy, sulfonate, sulfate, sulfite, and sulfide, or two geminal R⁶ and R⁷ groups can combine to form ═O or ═S;
each occurrence of Y is independently selected from the group consisting of C, O, N, S, P, and Si; and
x is 0, 1, 2, 3, 4, 5, 6, 7, 8, 9, or 10;
y is 0, 1, 2, 3, 4, 5, 6, 7, 8, 9, or 10;
each occurrence of z is 0, 1, 2, 3, 4, 5, 6, 7, 8, 9, 10, 11, 12, 13, 14, 15, 16, 17, 18, 19, or 20;
each occurrence of y′, z′ and z″ is independently 0, 1, or 2.

2. The compound of claim 1, wherein at least one of the following applies:
(a) each N atom in L is independently substituted with 0-2 instances of A;
(b) x is 0;

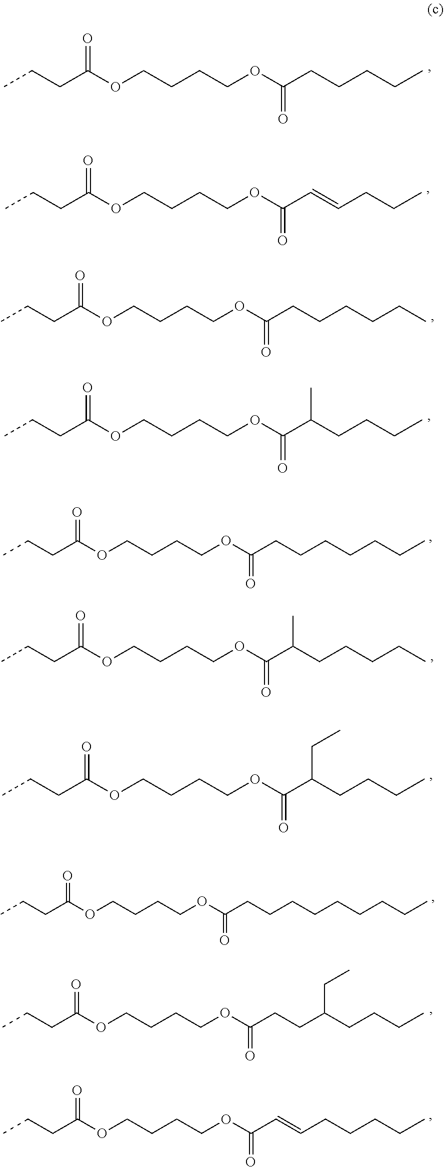

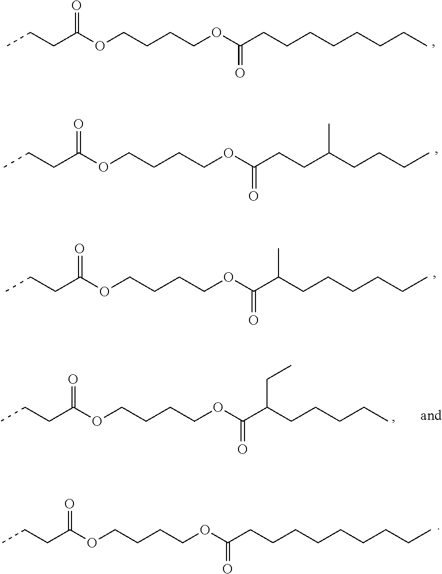

3. (canceled)

4. The compound of claim 1, wherein L is selected from the group consisting of: 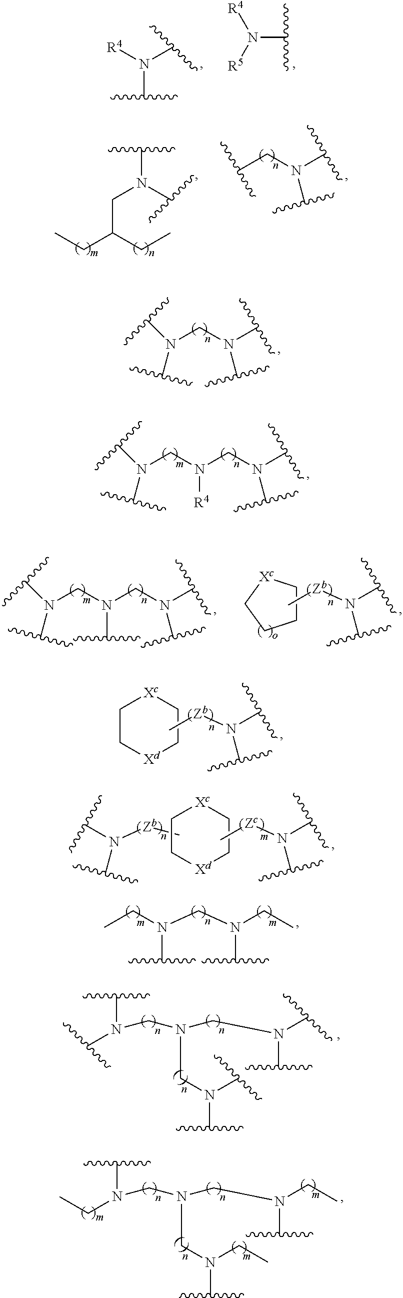 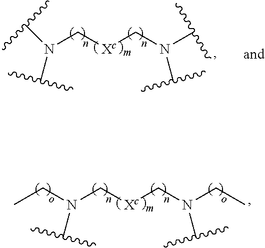
wherein:
R⁴ and R⁵, if present, are each independently selected from the group consisting of hydrogen, halogen, optionally substituted C₁-C₁₂ alkyl, optionally substituted C₃-C₁₂ cycloalkyl, optionally substituted —Y(R⁶)_{z′}(R⁷)_{z″}—(C₃-C₁₂ cycloalkyl), optionally substituted C₂-C₁₂ heterocycloalkyl, optionally substituted-(R⁶)_{z′}(R⁷)_{z″}—(C₂-C₁₂ heterocycloalkyl), optionally substituted C₂-C₁₂ alkenyl, optionally substituted C₅-C₁₂ cycloalkenyl, optionally substituted —Y(R⁶)_{z′}(R⁷)_{z″}—(C₅-C₁₂ cycloalkenyl), optionally substituted C₂-C₁₂ alkynyl, optionally substituted C₅-C₁₂ cycloalkynyl, optionally substituted —Y(R⁶)_{z′}(R⁷)_{z″}—(C₈-C₁₂ cycloalkynyl), optionally substituted C₆-C₁₀ aryl, optionally substituted —Y(R⁶)_{z′}(R⁷)_{z″}—(C₆-C₁₀ aryl), optionally substituted C₂-C₁₂ heteroaryl, optionally substituted —Y(R⁶)_{z′}(R⁷)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁶)_{z′}(R⁷)_{z″}-ester, —Y(R⁶)_{z′}(R⁷)_{z″}, —NO₂, —CN, sulfoxy, sulfonate, sulfate, sulfite, and sulfide;
each occurrence of X^{c} and X^{d} is independently selected from the group consisting of —O—, —S—, —N(R⁶)—, —P(R⁶)_{y′}—;
each occurrence of Z^{b} and Z° is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, and optionally substituted C₂-C₈ heterocyloalkylenyl;
each occurrence of m, n, and o is independently 0, 1, 2, 3, 4, 5, 6, 7, 8, 9, or 10; and
each occurrence of 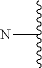 [SMILES: *N]
indicates a bond between a N atom and A or R¹, if present.

5. (canceled)

6. The compound of claim 1, wherein L is selected from the group consisting of: 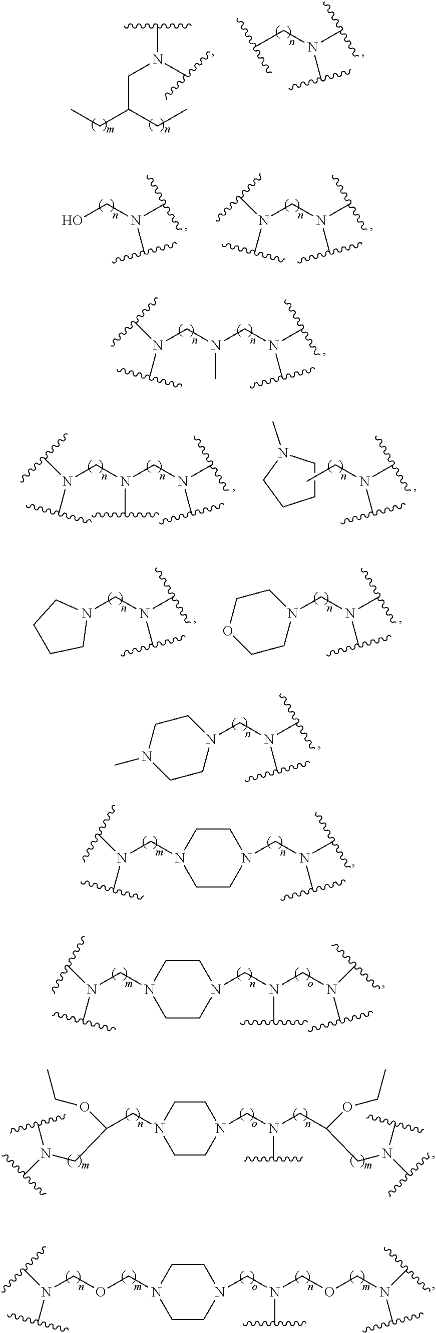
wherein:
each occurrence of m, n, and o is independently 0, 1, 2, 3, 4, 5, 6, 7, 8, 9, or 10; and
each occurrence of 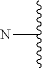 [SMILES: *N]
indicates a bond between a N atom and A or R¹, if present.

7. The compound of claim 1, which is selected from the group consisting of: 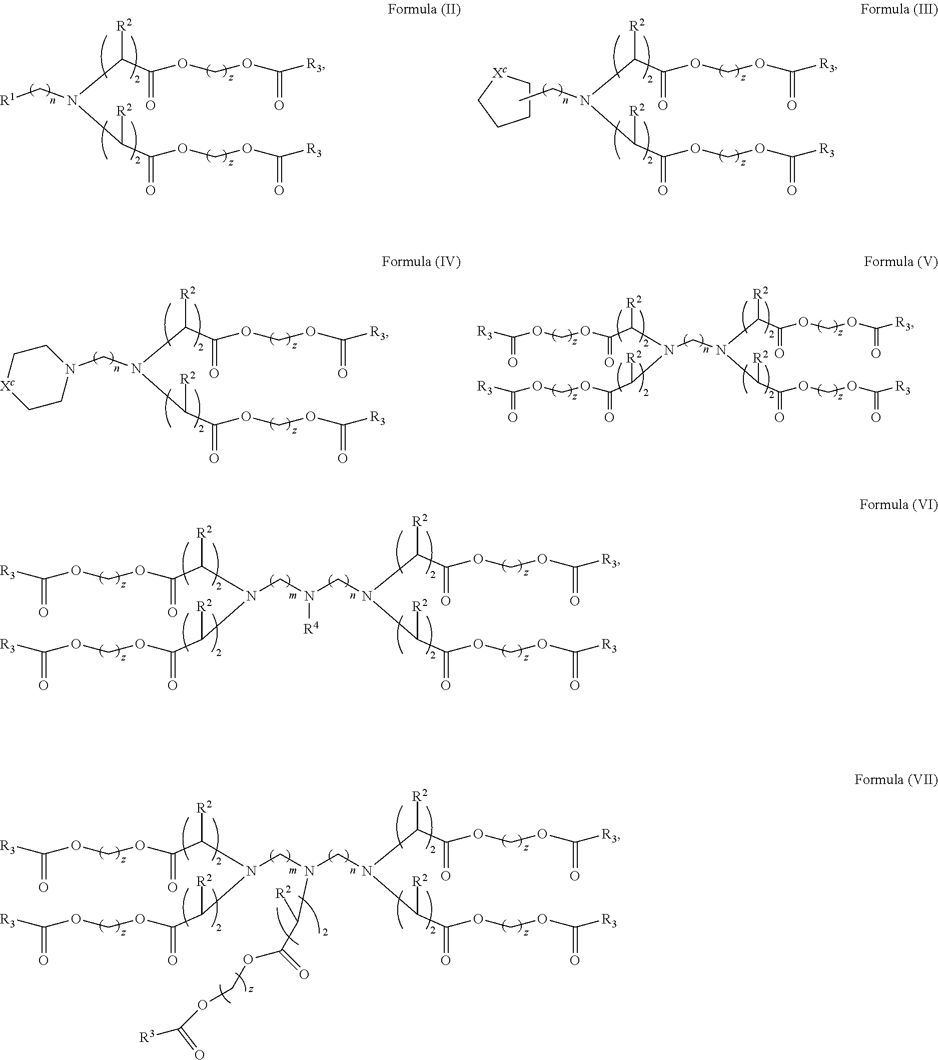 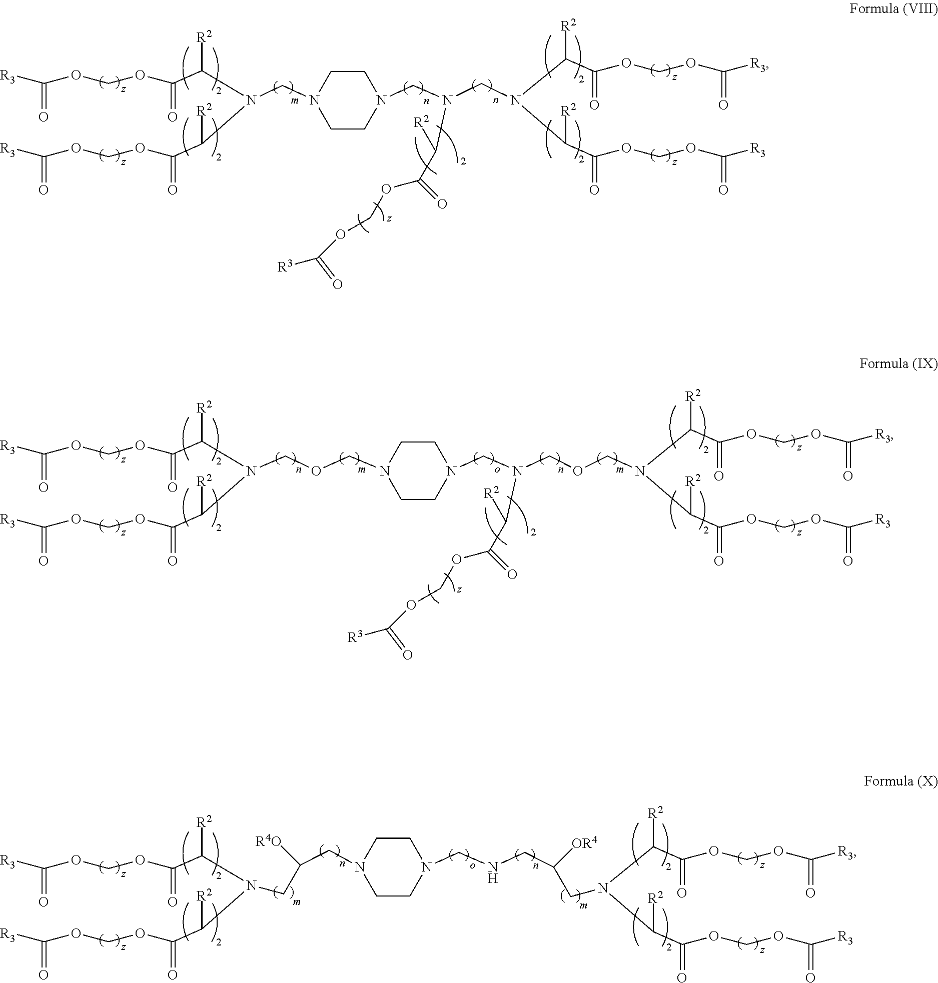
wherein:
each occurrence of X^{c} is independently selected from the group consisting of —O—, —S—, —N(R⁶)_{y′}—, and —P(R⁶)_{y′}—;
each occurrence of R⁴ is independently selected from the group consisting of hydrogen, halogen, optionally substituted C₁-C₁₂ alkyl, optionally substituted C₃-C₁₂ cycloalkyl, optionally substituted —Y(R⁶)_{z′}(R⁷)_{z″}—(C₃-C₁₂ cycloalkyl), optionally substituted C₂-C₁₂ heterocycloalkyl, optionally substituted-(R⁶)_{z′}(R⁷)_{z″}—(C₂-C₁₂ heterocycloalkyl), optionally substituted C₂-C₁₂ alkenyl, optionally substituted C₅-C₁₂ cycloalkenyl, optionally substituted —Y(R⁶)_{z′}(R⁷)_{z″}—(C₅-C₁₂ cycloalkenyl), optionally substituted C₂-C₁₂ alkynyl, optionally substituted C₈-C₁₂ cycloalkynyl, optionally substituted —Y(R⁶)_{z′}(R⁷)_{z″}—(C₈-C₁₂ cycloalkynyl), optionally substituted C₆-C₁₀ aryl, optionally substituted —Y(R⁶)_{z′}(R⁷)_{z″}—(C₆-C₁₀ aryl), optionally substituted C₂-C₁₂ heteroaryl, optionally substituted —Y(R⁶)_{z′}(R⁷)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁶)_{z′}(R⁷)_{z″}-ester, —Y(R⁶)_{z′}(R⁷)_{z″}, —NO₂, —CN, sulfoxy, sulfonate, sulfate, sulfite, and sulfide; and
each occurrence of m, n, and o is independently 0, 1, 2, 3, 4, 5, 6, 7, 8, 9, or 10.

8. The compound of claim 7, wherein at least one of the following applies:
(a) each occurrence of R¹, R³, and R⁴ is alkyl or substituted alkyl;
(b) each occurrence of R² is hydrogen;
(c) each occurrence of X^{c} is O or N(R⁶)_{y′}, wherein R⁶ is alkyl and y′ is 1;
(d) each occurrence of m and o is an integer represented by 2;
(e) each occurrence of n is an integer represented by 2 or 3; and
(f) each occurrence of z is an integer represented by 4.

9. The compound of claim 1, wherein at least one of the following applies:
(a) each occurrence of R² is H;
(b) each occurrence of Z^{a} is —CH₂—;
(c) each occurrence of z is an integer represented by 4;
(d) each occurrence of X^{a} is O;
(e) each occurrence of X^{b} is O;
(f) x is an integer represented by 0;
(g) R¹ is absent;
(h) y is an integer represented by 2, 3, 4, or 5; and
(i) each occurrence of R³ is independently selected from the group consisting of n-pentyl, n-hexyl, n-heptyl, n-octyl, n-nonyl, 1-pentenyl, 1-heptenyl, 1-methylpentyl, 1-methylhexyl, 1-methylheptyl, 1-ethylpentyl, 2-ethylhexyl, and 3-methylheptyl, optionally wherein each occurrence of R³ is identical.

10. The compound of claim 1, which is selected from the group consisting of:

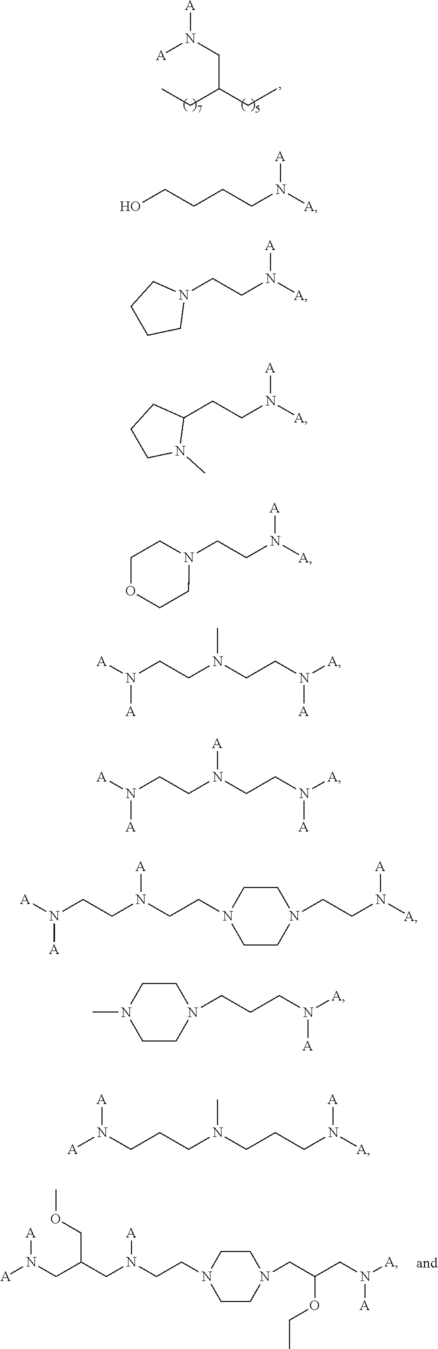

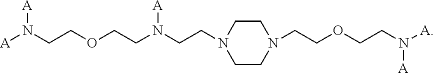

11. (canceled)

12. The compound of claim 1, wherein the compound of Formula (I) is selected from the group consisting of:

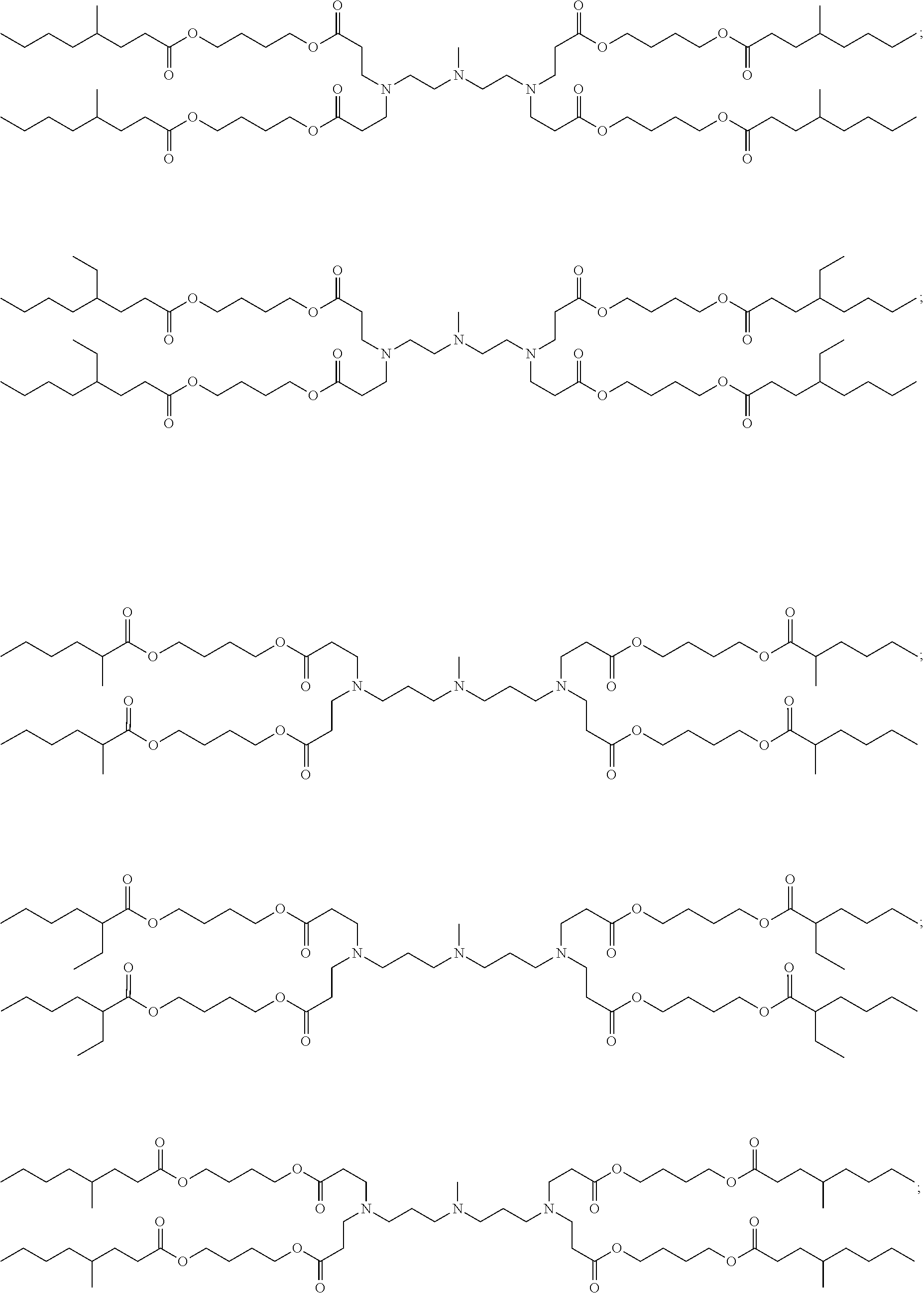

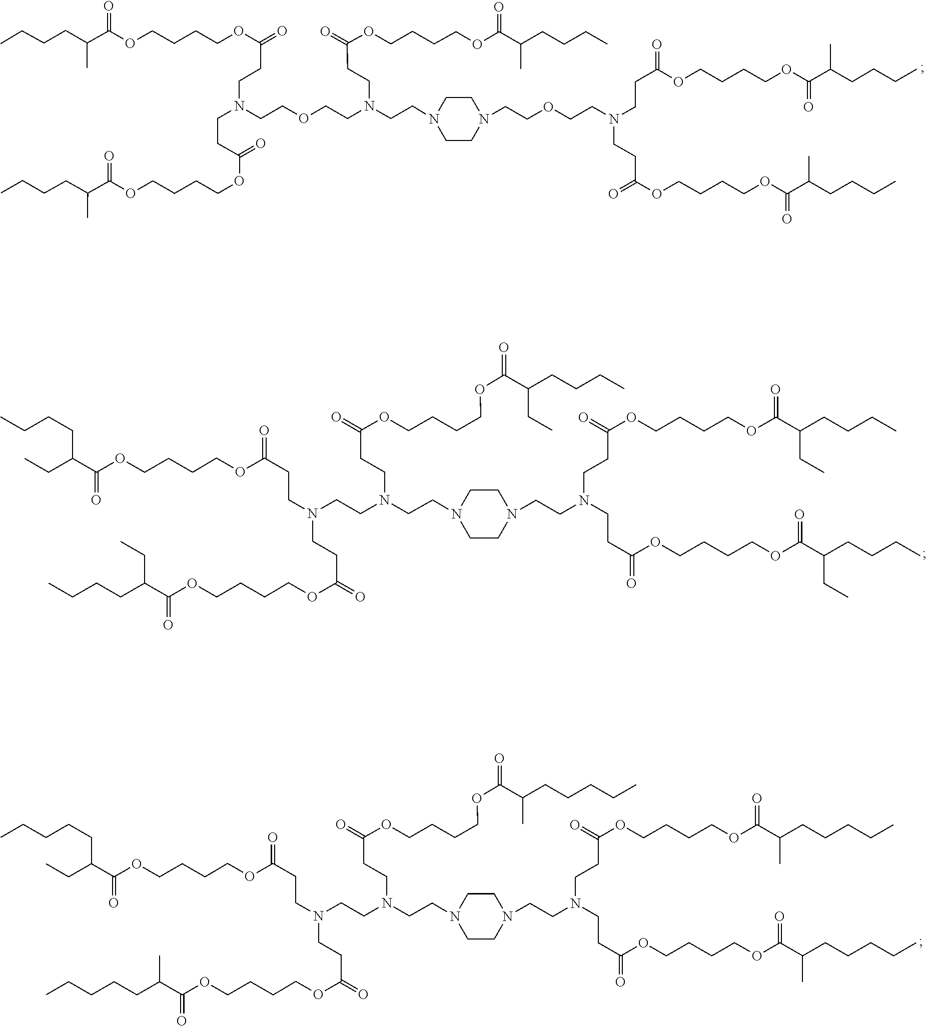

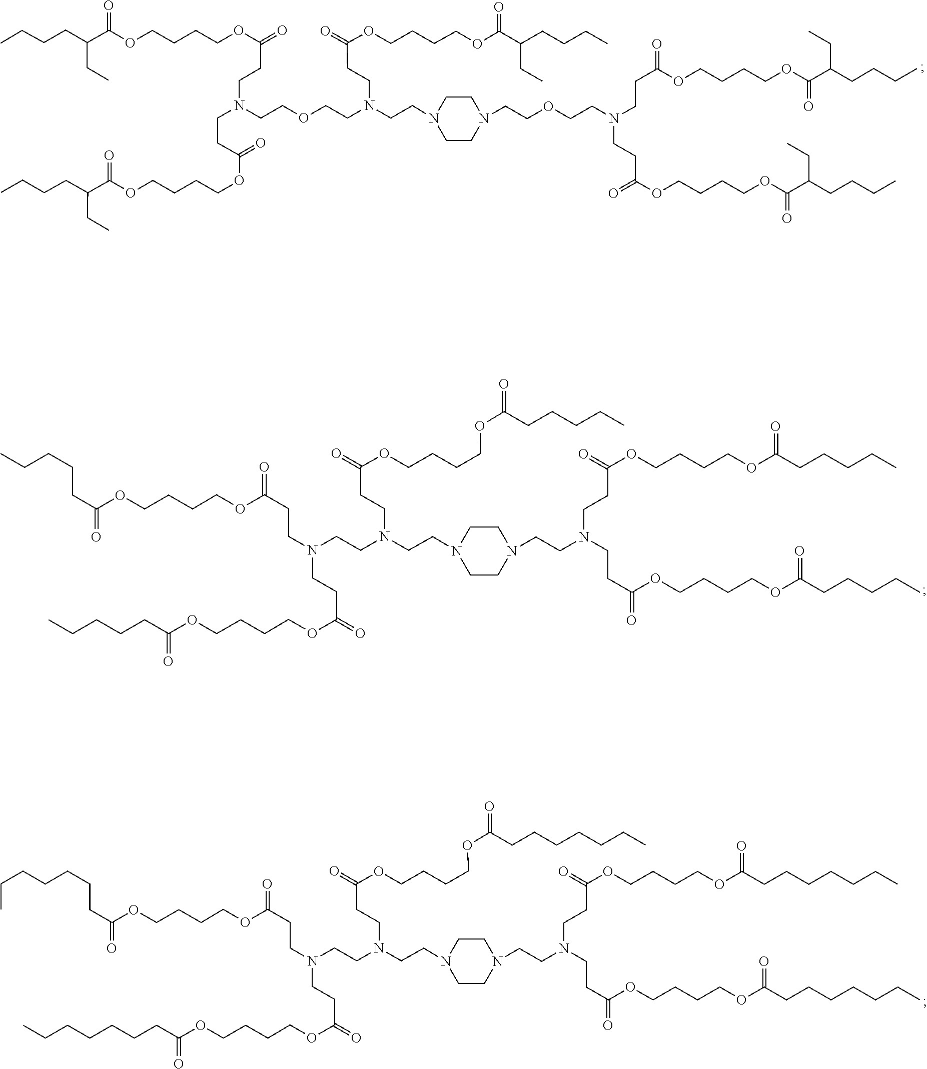

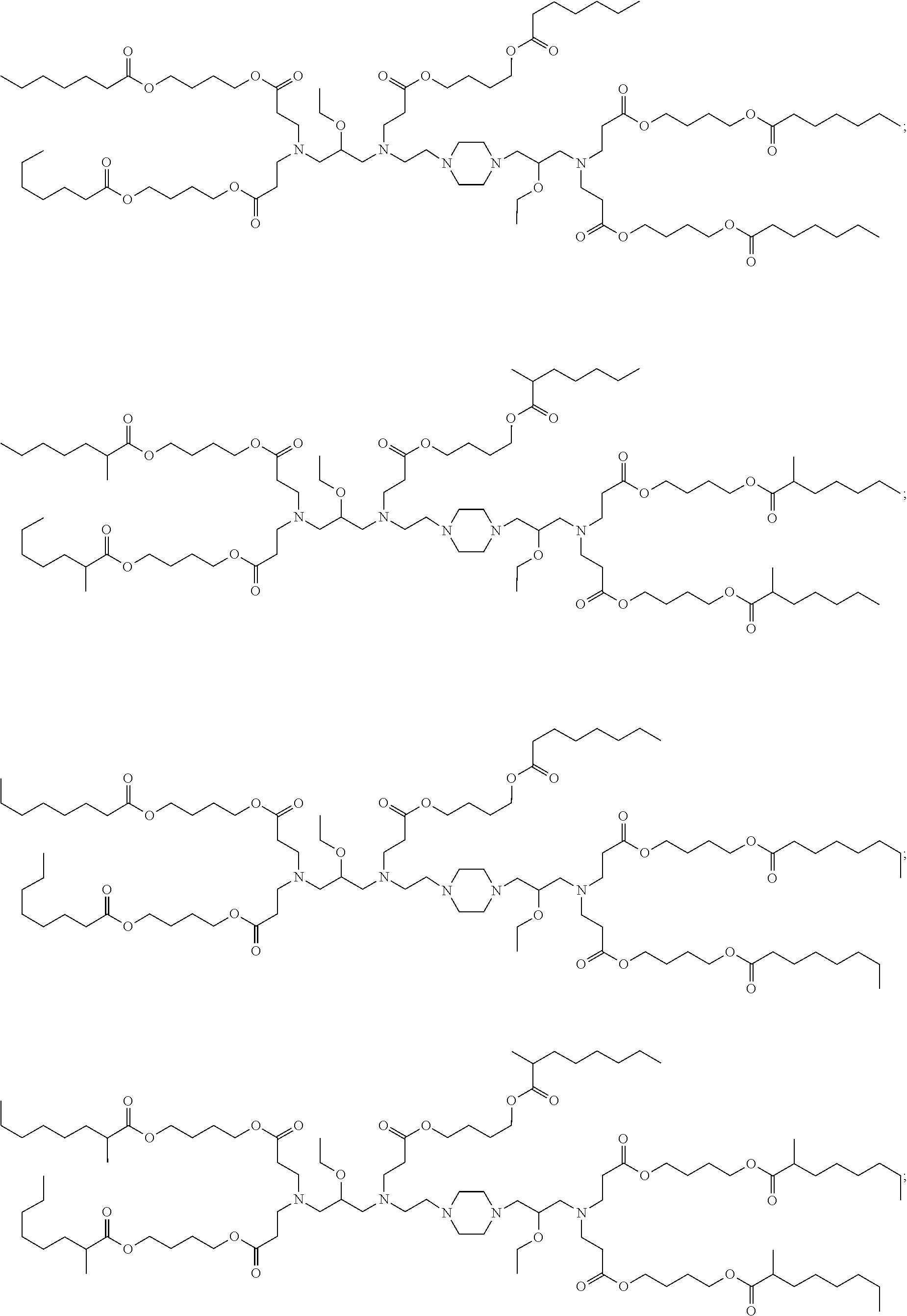

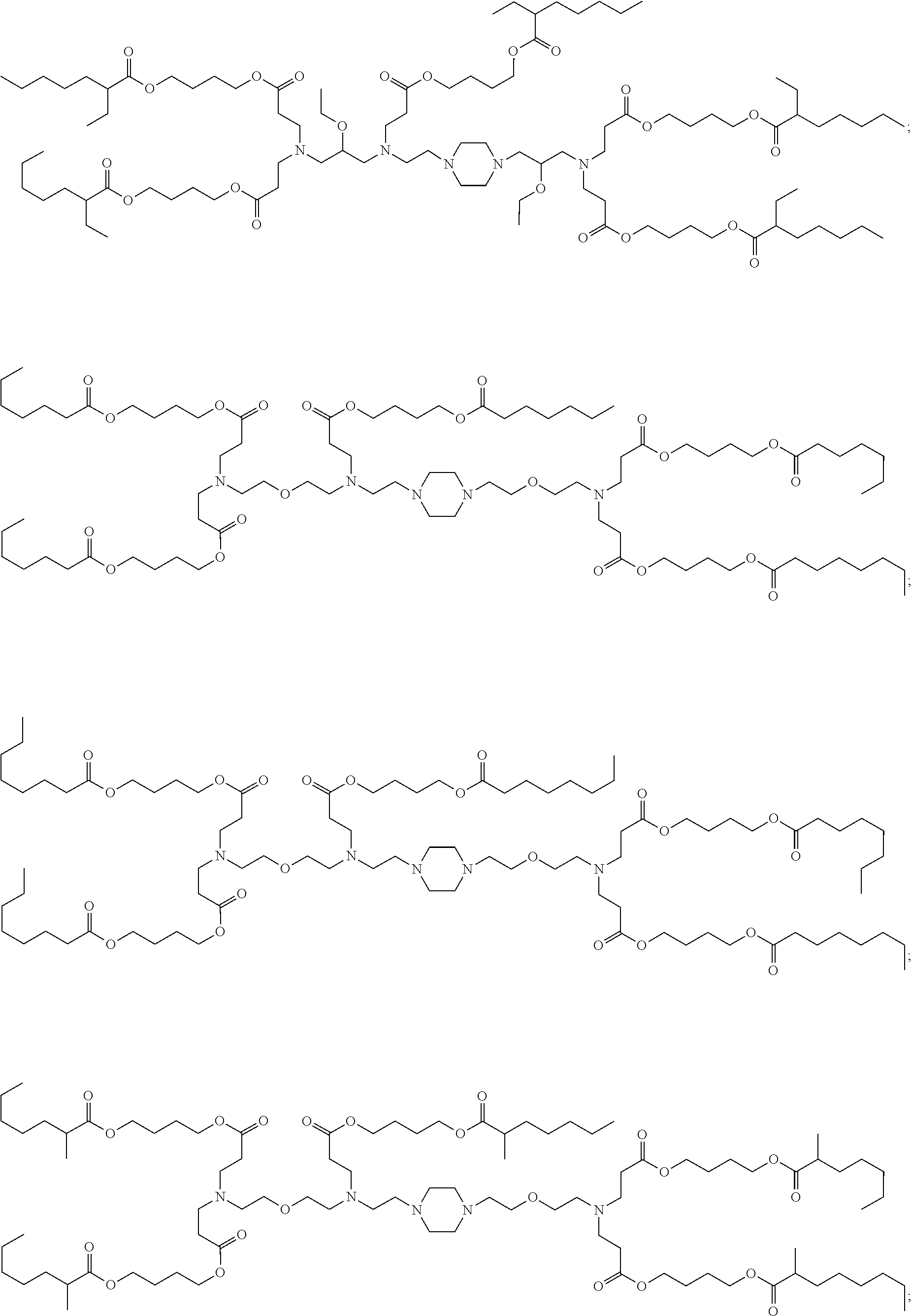

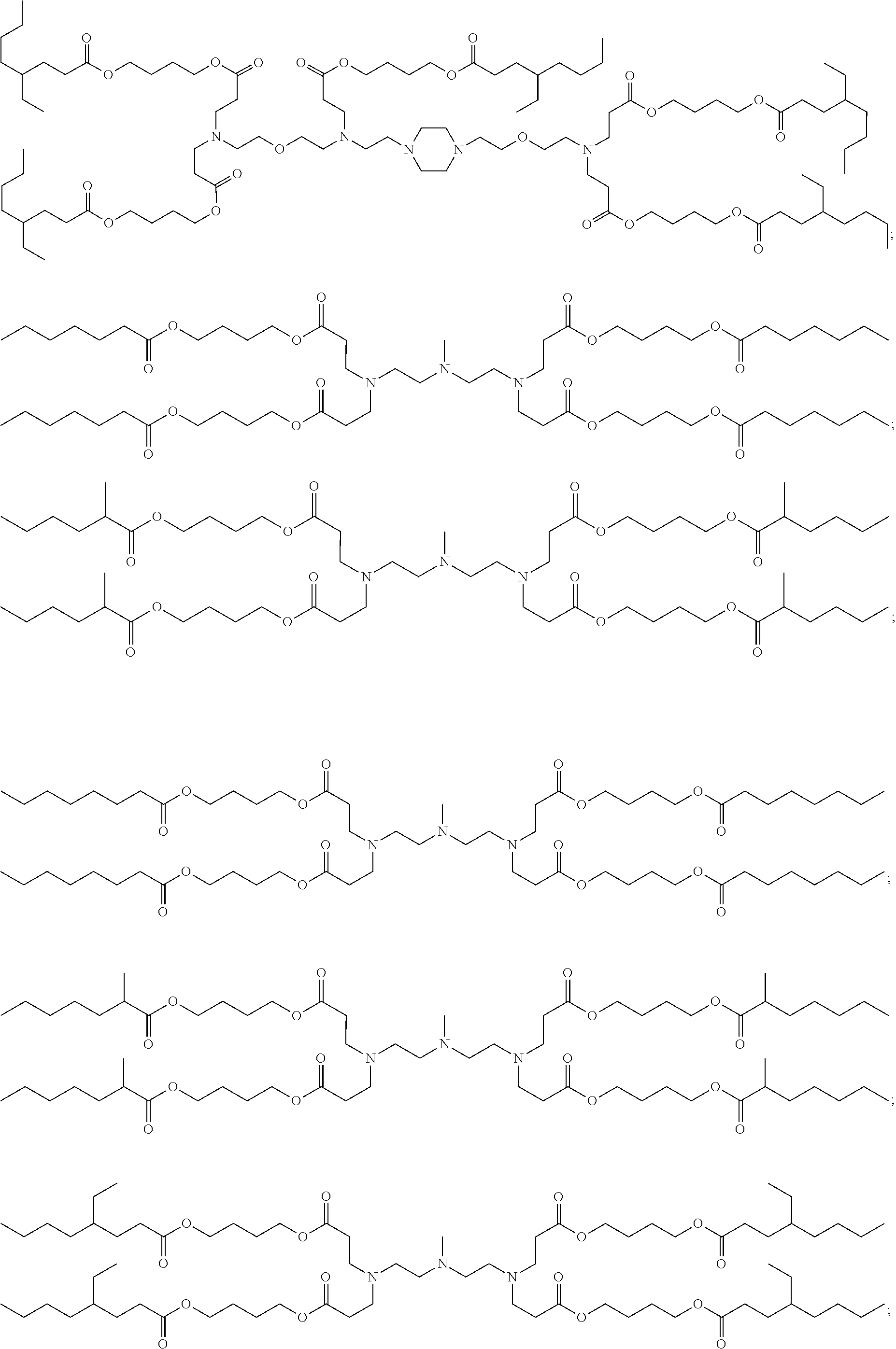

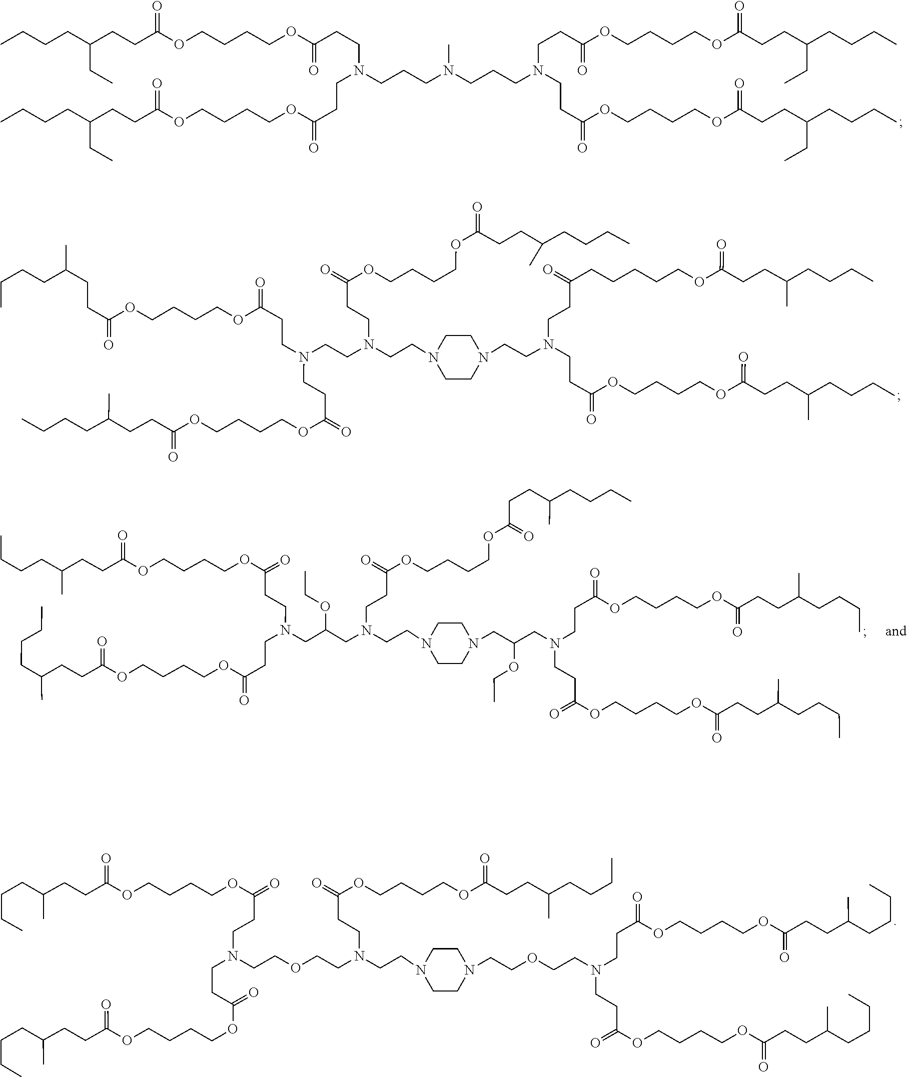

13. (canceled)

14. A biodegradable lipid nanoparticle (LNP) comprising:
(a) at least one compound of claim 1;
(b) at least one neutral phospholipid, wherein the neutral phospholipid is present in a concentration range of about 5 mol % to about 45 mol %;
(c) at least one cholesterol lipid, wherein the total cholesterol lipid is in a concentration range of about 5 mol % to about 55 mol %; and
(d) at least one polyethylene glycol (PEG) or PEG-conjugated lipid, wherein the PEG or PEG-conjugated lipid is in a concentration range of about 0.5 mol % to about 12.5 mol %.

15. The biodegradable LNP of claim 14, wherein at least one of the following applies:
(a) the at least one neutral phospholipid comprises at least one selected from the group consisting of dioleoyl-phosphatidylethanolamine (DOPE), dioleoylphosphatidylcholine (DOPC), distearoylphosphatidylcholine (DSPC), distearoyl-phosphatidylethanolamine (DSPE), 16-O-dimethyl PE, 18-1-trans PE, 1-stearioyl-2-oleoyl-phosphatidyethanol amine (SOPE), stearoyloleoylphosphatidylcholine (SOPC), N-(2,3-dioleoyloxy)propyl)-N,N,N-trimethylammonium chloride (DOTAP), and any combination thereof;
(b) the at least one cholesterol lipid comprises at least one selected from the group consisting of a cholesterol, cholesterol derivate, and any combination thereof;
(c) the at least one PEG or PEG-conjugated lipid comprises at least one selected from the group consisting of 1,2-dimyristoyl-sn-glycero-3-phosphoethanolamine-N-[methoxy(polyethylene glycol)-2000](C₁₄-PEG2000), C₁₂-PEG2000, C₁₂-PEG490, 1,2-dimyristoyl-rac-glycero-3-methoxypolyethylene glycol-2000 (DMG-PEG2000), 1,2-distearoyl-sn-glycero-3-phosphoethanolamine-N-[amino(polyethylene glycol)-2000](DSPE-PEG 2000 amine), and any combination thereof;
(d) the molar ratio of (a):(b):(c):(d) is about 1-80:5-45:5-55:0.5-12.5, optionally wherein, optionally wherein the molar ratio of (a):(b):(c):(d) is about 25-45:5-20:40-55:1-2.5, and optionally wherein the molar ratio of (a):(b):(c):(d) is selected from the group consisting of about 30:16:46.5:2.5, 31:16:46.5:2.5, 32:16:46.5:2.5, 33:16:46.5:2.5, 34:16:46.5:2.5, and 35:16:46.5:2.5;
(e) the biodegradable LNP has a diameter of between about 50 nm to about 500 nm, optionally wherein the biodegradable LNP has a diameter of between about 50 nm to about 160 nm; and
(f) the biodegradable LNP selectively binds to at least one target cell of interest, optionally wherein the at least one target cell of interest is selected from the group consisting of an immune cell, stem cell, bone cell, blood cell, fat cell, endothelial cell, cancer cell, tissue cell, nerve cell, epithelial cell, connective tissue cell, muscle cell, and any combination thereof, and optionally wherein the at least one target cell of interest is selected from the group consisting of a brain cell, neuron, neuroglial cell, heart cell, liver cell, spleen cell, lung cell, kidney cell, podocytes, skin cell, keratinocyte, melanocyte, merkel cell, langerhans cell, cartilage cell, chondrocyte, pancreatic cell, skeletal muscle cell, cardiac muscle cell, smooth muscle cell, bone cell, osteoblast, osteoclast, osteocyte, lining cell, bone marrow cell, lymph node cell, white blood cell, granulocyte, neutrophil, eosinophil, basophil, agranulocyte, monocyte, lymphocyte, red blood cell, erythrocyte, platelet, fragments of megakarvocyte, embryonic stem cell, adult stem cell, mesenchymal stem cell, hematopoietic stem cell, white adipocyte, brown adipocyte, and any combination thereof.

16-25. (canceled)

26. The biodegradable LNP of claim 14, wherein the biodegradable LNP further comprises or encapsulates at least one agent and at least one of the following applies:
(a) the compound (a) and at least one agent have a weight ratio between about 1:1 to about 10:1;
(b) the agent is selected from the group consisting of a nucleic acid molecule, a small molecule, a protein, an antibody, and any combination thereof, optionally wherein the nucleic acid molecule is a DNA molecule or an RNA molecule, optionally wherein the nucleic acid molecule is selected from the group consisting of cDNA, mRNA, miRNA, siRNA, sgRNA, modified RNA, antagomir, antisense molecule, targeted nucleic acid, and any combination thereof; and
(c) the biodegradable LNP is suitable for delivering a nucleic acid to a liver cell.

27-31. (canceled)

32. A composition comprising at least one biodegradable LNP of claim 14, optionally wherein the composition further comprises at least one pharmaceutically acceptable carrier, and optionally wherein the composition is suitable for delivering a nucleic acid to a liver cell.

33-34. (canceled)

35. A method of delivering an agent to a subject in need thereof, the method comprising administering a therapeutically effectively amount of at least one biodegradable LNP of claim 14 or a composition comprising the same to the subject.

36. The method of claim 35, wherein at least one of the following applies:
(a) the agent is selected from the group consisting of a nucleic acid molecule, a small molecule, a protein, an antibody, a therapeutic agent, and any combination thereof, optionally wherein the nucleic acid molecule is a DNA molecule or an RNA molecule, and optionally wherein the nucleic acid molecule is selected from the group consisting of cDNA, mRNA, miRNA, siRNA, sgRNA, modified RNA, antagomir, antisense molecule, targeted nucleic acid, and any combination thereof;
(b) the biodegradable LNP selectively binds to at least one target cell of interest, optionally wherein the at least one target cell of interest is selected from the group consisting of an immune cell, stem cell, bone cell, blood cell, fat cell, endothelial cell, cancer cell, tissue cell, nerve cell, epithelial cell, connective tissue cell, muscle cell, and any combination thereof, and optionally wherein the at least one target cell of interest is selected from the group consisting of a brain cell, neuron, neuroglial cell, heart cell, liver cell, spleen cell, lung cell, kidney cell, podocytes, skin cell, keratinocyte, melanocyte, merkel cell, langerhans cell, cartilage cell, chondrocyte, pancreatic cell, skeletal muscle cell, cardiac muscle cell, smooth muscle cell, bone cell, osteoblast, osteoclast, osteocyte, lining cell, bone marrow cell, lymph node cell, white blood cell, granulocyte, neutrophil, eosinophil, basophil, agranulocyte, monocyte, lymphocyte, red blood cell, erythrocyte, platelet, fragments of megakarvocyte, embryonic stem cell, adult stem cell, mesenchymal stem cell, hematopoietic stem cell, white adipocyte, brown adipocyte, and any combination thereof;
(c) the at least one target cell of interest is a liver cell;
(d) the agent is encapsulated within the biodegradable LNP; and
(e) the biodegradable LNP or the composition comprising the same is administered intravenously, intramuscularly, intradermally, subcutaneously, intranasally, by inhalation, or any combination thereof.

37-44. (canceled)

45. A method of delivering an agent to a liver, the method comprising administering a therapeutically effectively amount of at least one biodegradable LNP of claim 14 or a composition comprising the same.

46. The method of claim 45, wherein at least one of the following applies:
(a) the agent is selected from the group consisting of a nucleic acid molecule, a small molecule, a protein, an antibody, a therapeutic agent, and any combination thereof, optionally wherein the nucleic acid molecule is a DNA molecule or an RNA molecule, and optionally wherein the nucleic acid molecule is selected from the group consisting of cDNA, mRNA, miRNA, siRNA, sgRNA, modified RNA, antagomir, antisense molecule, targeted nucleic acid, and any combination thereof;
(b) the at least one target cell of interest is a liver cell;
(c) the agent is encapsulated within the biodegradable LNP; and
(d) the biodegradable LNP or the composition comprising the same is administered intravenously, intramuscularly, intradermally, subcutaneously, intranasally, by inhalation, or any combination thereof.

47-51. (canceled)

52. A method of treating, ameliorating, and/or preventing at least one disease or disorder in a subject in need thereof, the method comprising administering a therapeutically effectively amount of at least one biodegradable LNP of claim 14 or a composition comprising the same to the subject.

53. The method of claim 52, wherein the disease or disorder is selected from the group consisting of liver disease or disorder, pulmonary disease or disorder, spleen disease or disorder, renal disease or disorder, heart disease or disorder, cardiovascular disease or disorder, brain disease or disorder, neurological disease or disorder, cancer, bone disease or disorder, bone marrow disease or disorder, skin disease or disorder, connective tissue disease or disorder, pancreatic disease or disorder, muscle disease or disorder, lymph node disease or disorder, blood disease or disorder, and any combination thereof.

54. A method of inducing an immune response in a subject in need thereof, the method comprising administering a therapeutically effectively amount of at least one biodegradable LNP of claim 14 or a composition comprising the same to the subject.
